SEQUENCE LISTING (12 sequences)

SEQ ID NO: 1 - protein, 518 aa, Artificial Sequence
  Homo sapiens motile sperm domain containing 2 (MOSPD2), protein variant 1
MAENHAQNKAKLISETRRRFEAEYVTDKSDKYDARDVERLQQDDNWVESYLSWRHNIVDETLKMLDESFQWRKEISVNDLNESSIPRWLLEIGVIYLHGYDKEGNKLFWIRVKYHVKDQKTILDKKKLIAFWLERYAKRENGKPVTVMFDLSETGINSIDMDFVRFIINCFKVYYPKYLSKIVIFDMPWLMNAAFKIVKTWLGPEAVSLLKFTSKNEVQDYVSVEYLPPHMGGTDPFKYSYPPLVDDDFQTPLCENGPITSEDETSSKEDIESDGKETLETISNEEQTPLLKKINPTESTSKAEENEKVDSKVKAFKKPLSVFKGPLLHISPAEELYFGSTESGEKKTLIVLTNVTKNIVAFKVRTTAPEKYRVKPSNSSCDPGASVDIVVSPHGGLTVSAQDRFLIMAAEMEQSSGTGPAELTQFWKEVPRNKVMEHRLRCHTVESSKPNTLTLKDNAFNMSDKTSEDICLQLSRLLESNRKLEDQVQRCIWFQQLLLSLTMLLLAFVTSFFYLLYS

SEQ ID NO: 2 - protein, 455 aa, Artificial Sequence
  Homo sapiens motile sperm domain containing 2 (MOSPD2), protein variant 2
MLDESFQWRKEISVNDLNESSIPRWLLEIGVIYLHGYDKEGNKLFWIRVKYHVKDQKTILDKKKLIAFWLERYAKRENGKPVTVMFDLSETGINSIDMDFVRFIINCFKVYYPKYLSKIVIFDMPWLMNAAFKIVKTWLGPEAVSLLKFTSKNEVQDYVSVEYLPPHMGGTDPFKYSYPPLVDDDFQTPLCENGPITSEDETSSKEDIESDGKETLETISNEEQTPLLKKINPTESTSKAEENEKVDSKVKAFKKPLSVFKGPLLHISPAEELYFGSTESGEKKTLIVLTNVTKNIVAFKVRTTAPEKYRVKPSNSSCDPGASVDIVVSPHGGLTVSAQDRFLIMAAEMEQSSGTGPAELTQFWKEVPRNKVMEHRLRCHTVESSKPNTLTLKDNAFNMSDKTSEDICLQLSRLLESNRKLEDQVQRCIWFQQLLLSLTMLLLAFVTSFFYLLYS

SEQ ID NO: 3 - protein, 486 aa, Artificial Sequence
  Homo sapiens motile sperm domain containing 2 (MOSPD2), protein variant 3
MAENHAQNKAKLISETRRRFEAEYVTDKSDKYDARDVERLQQDDNWVESYLSWRHNIVDETLKMLDESFQWRKEISVNDLNESSIPRWLLEIGVIYLHGYDKEGNKLFWIRVKYHVKDQKTILDKKKLIAFWLERYAKRENGKPVTVMFDLSETGINSIDMDFVRFIINCFKVYYPKYLSKIVIFDMPWLMNAAFKIVKTWLGPEAVSLLKFTSKNEVQDYVSVEYLPPHMGGTDPFKYSYPPLVDDDFQTPLCENGPITSEDETSSKEDIESDGKETLETISNEEQTPLLKKINPTESTSKAEENEKVDSKVKAFKKPLSVFKGPLLHISPAEELYFGSTESGEKKTLIVLTNVTKNIVAFKVRTTAPEKYRVKPSNSSCDPGASVDIVVSPHGGLTVSAQDRFLIMAAEMEQSSGTGPAELTQFWKEVPRNKVMEHRLRCHTVESSKPNTLTLKDNAFNMSDKTSEDICLQFATSSCEMDCSPP

SEQ ID NO: 4 - protein, 475 aa, Artificial Sequence
  Homo sapiens motile sperm domain containing 2 (MOSPD2), protein variant X2
MAENHAQNKAKLISETRRRFEAEYVTDKSDKYDARDVERLQQDDNWVESYLSWRHNIVDETLKMLDESFQWRKEISVNDLNESSIPRWLLEIGVIYLHGYDKEGNKLFWIRVKYHVKDQKTILDKKKLIAFWLERYAKRENGKPVTVMFDLSETGINSIDMDFVRFIINCFKVYYPKYLSKIVIFDMPWLMNAAFKIVKTWLGPEAVSLLKFTSKNEVQDYVSVEYLPPHMGGTDPFKYSYPPLVDDDFQTPLCENGPITSEDETSSKEDIESDGKETLETISNEEQTPLLKKINPTESTSKAEENEKVDSKVKAFKKPLSVFKGPLLHISPAEELYFGSTESGEKKTLIVLTNVTKNIVAFKVRTTAPEKYRVKPSNSSCDPGASVDIVVSPHGGLTVSAQDRFLIMAAEMEQSSGTGPAELTQFWKEVPRNKVMEHRLRCHTVESSKPNTLTLKDNAFNMSDKTSEDICLQYS

SEQ ID NO: 5 - DNA, 3218 bp, Artificial Sequence
  Homo sapiens motile sperm domain containing 2 (MOSPD2), transcript variant 1
accgcctccccctcccacccttctctgtctacctctgggcggactgccgggtgatgagatactcggtcggcgacggtagaacgggcgacggcgacaaccgcaatcacatccacgacggtgatcatggcagagaatcacgcccagaataaagccaagctcatctctgagacccggaggaggttcgaagctgagtatgtgacagataagtcagataaatatgatgcacgtgatgttgaaaggctacaacaagatgataactgggttgaaagttacttatcttggagacataatattgtagatgaaacactgaagatgctcgatgagagttttcagtggaggaaagaaatttctgtcaatgaccttaatgaatcctccattcccagatggttattggaaattggtgttatttatctccatggttatgacaaagaaggtaacaaattgttctggatcagggtgaagtatcatgtaaaagaccagaaaaccatattggacaaaaagaagctcatagcattctggttggaacgttatgctaagagggaaaatgggaaacctgtaacagtgatgtttgacctgtcagaaactggaataaatagcattgacatggactttgtacgctttatcatcaactgctttaaggtttattaccctaaatacctctcaaaaatagtgatctttgatatgccttggttaatgaatgctgctttcaaaattgtgaaaacctggcttggtccagaagcagtgagcttgttgaagtttacaagcaaaaatgaagtccaggactatgtcagtgtagaatacctgcctccccacatgggtggaactgatcctttcaagtatagctatccaccactagtagatgatgacttccagaccccactgtgtgagaatgggcctattaccagtgaggatgaaacttcaagtaaagaagacatagaaagtgatggcaaagaaacattggaaacaatttctaatgaagaacaaacacctcttcttaaaaagattaacccaaccgaatctacttccaaagcagaagaaaatgaaaaagttgattcaaaagtgaaagctttcaagaaaccattgagtgtatttaaaggccccttactacacatcagcccagcagaagaactgtactttggaagtacagaatccggagagaagaaaaccttaatagtgttgacaaatgtaactaaaaatatagtggcatttaaggtgagaacaacagctccagaaaaatacagagtcaagccaagcaatagcagctgtgacccgggtgcatcagtggatatagttgtgtctccccatgggggtttaacagtctctgcccaagaccgttttctgataatggctgcagaaatggaacagtcatctggcacaggcccagcagaattaactcagttttggaaagaagttcccagaaacaaagtgatggaacataggttaagatgccatactgttgaaagcagtaaaccaaacactcttacgttaaaagacaatgctttcaatatgtcagataaaaccagtgaagatatatgtctacaactcagtcgtttactagaaagcaataggaagcttgaagaccaagttcagcgttgtatctggttccagcagctgctgctttccttaacaatgctcttgcttgcttttgtcacctctttcttctatttattgtacagttaaagaagtggtgccgggtaggaaccacggttccttcgtccattagttggaaaaagtaacagacctaaaactctaccaagctactaaaaacattgcacatctgtgcttcctaaaaggaaatatgcagcacgtggaggggaacacatacatgtcttgaaaataaactgctagaataaagaaatgctggagaaattgattataagagactatagctatttagtaaagtaagtaaaggcatatccattgtgtaaattaatagtttaaatataatttattttttccttttgatctgaatacttttaaagcttaagttttatcgtgtaaatacattagctaaactgaaaagtataagtaacatgctttgttgcagccaaaaaatgtaatctgcttttttatgacagaattattatagctgagctgacttactagcttttctatactatgtatatagaagaacatgtatattgagaaagaaaacatacttatatagaggaatttatgtaaccatgactttgtaattttgagaattcctcccagtgatggtcagtattcttttggaatgtaaaccgatttaatgccaaaccaccttaacctttgtttctcagtgttccttaacagcctgccttttattaatctcaggcttttttatgaacactctcatttcagtagaatttggaaaactaagcgtggttggaatttctttgaattctgttagtaatgcccaaaagaaaagtctcaagcagtccccctatccagtcatttttatggagtttcatgttgtccactatagctggacactgaaccttttgcctaatttattataaaggcctgaccctctattgtcccatcttcacccccattccagagcagaggagtctctgtggaccatgaattgcactgtctccctcctcatttctaaatgaaaggtattagatataaatttttttgaaaggttagttgtttgagatgctaagcaggataataaatttagattttaaaatgttccctgtaaaagtcagcccatgacaaggaaatttacaaaatactagagtatctagaagggtgaaaacaaaaaaaaataaaaagaaacacagacgcccaggtgtcagctctccgtttaaagaatgaaaaatgtaactcatgatgatctgtgaaaccttcaaactaggaccaattgacttacttgatattctgcctttgatatggtagtacccacccggtattcctaaaatcctaaaaagatacaccttgcagtagcagaggcaatgacatgagtttgttttctcattaatatgaccagtttgggtctatgttggttcacatgtacatctactttatatgaaagaaaaaacagttgtctgcctgtaaaatgttgagtttcgattgagccatgtttggagattttattactattctgaagggtagtgttgttggttttcatcttcaagaagttgattccaaaactgagttatgaagaatgatataacagttccttcaaaattggcctaggaaataaaaccttaaaaggacaaaaaaaaaaa

SEQ ID NO: 6 - DNA, 3183 bp, Artificial Sequence
  Homo sapiens motile sperm domain containing 2 (MOSPD2), transcript variant 2
agtcacaataataggtacttaaaaacatgtcatatgataggaaactagaataacacacctctaaataatccatggattaccaacttctcagcaatggtgtaaattcttctgctaaataacccgtgggttaaagaggacatcacattggaaattccagagtacttaaaataagtcagataaatatgatgcacgtgatgttgaaaggctacaacaagatgataactgggttgaaagttacttatcttggagacataatattgtagatgaaacactgaagatgctcgatgagagttttcagtggaggaaagaaatttctgtcaatgaccttaatgaatcctccattcccagatggttattggaaattggtgttatttatctccatggttatgacaaagaaggtaacaaattgttctggatcagggtgaagtatcatgtaaaagaccagaaaaccatattggacaaaaagaagctcatagcattctggttggaacgttatgctaagagggaaaatgggaaacctgtaacagtgatgtttgacctgtcagaaactggaataaatagcattgacatggactttgtacgctttatcatcaactgctttaaggtttattaccctaaatacctctcaaaaatagtgatctttgatatgccttggttaatgaatgctgctttcaaaattgtgaaaacctggcttggtccagaagcagtgagcttgttgaagtttacaagcaaaaatgaagtccaggactatgtcagtgtagaatacctgcctccccacatgggtggaactgatcctttcaagtatagctatccaccactagtagatgatgacttccagaccccactgtgtgagaatgggcctattaccagtgaggatgaaacttcaagtaaagaagacatagaaagtgatggcaaagaaacattggaaacaatttctaatgaagaacaaacacctcttcttaaaaagattaacccaaccgaatctacttccaaagcagaagaaaatgaaaaagttgattcaaaagtgaaagctttcaagaaaccattgagtgtatttaaaggccccttactacacatcagcccagcagaagaactgtactttggaagtacagaatccggagagaagaaaaccttaatagtgttgacaaatgtaactaaaaatatagtggcatttaaggtgagaacaacagctccagaaaaatacagagtcaagccaagcaatagcagctgtgacccgggtgcatcagtggatatagttgtgtctccccatgggggtttaacagtctctgcccaagaccgttttctgataatggctgcagaaatggaacagtcatctggcacaggcccagcagaattaactcagttttggaaagaagttcccagaaacaaagtgatggaacataggttaagatgccatactgttgaaagcagtaaaccaaacactcttacgttaaaagacaatgctttcaatatgtcagataaaaccagtgaagatatatgtctacaactcagtcgtttactagaaagcaataggaagcttgaagaccaagttcagcgttgtatctggttccagcagctgctgctttccttaacaatgctcttgcttgcttttgtcacctctttcttctatttattgtacagttaaagaagtggtgccgggtaggaaccacggttccttcgtccattagttggaaaaagtaacagacctaaaactctaccaagctactaaaaacattgcacatctgtgcttcctaaaaggaaatatgcagcacgtggaggggaacacatacatgtcttgaaaataaactgctagaataaagaaatgctggagaaattgattataagagactatagctatttagtaaagtaagtaaaggcatatccattgtgtaaattaatagtttaaatataatttattttttccttttgatctgaatacttttaaagcttaagttttatcgtgtaaatacattagctaaactgaaaagtataagtaacatgctttgttgcagccaaaaaatgtaatctgcttttttatgacagaattattatagctgagctgacttactagcttttctatactatgtatatagaagaacatgtatattgagaaagaaaacatacttatatagaggaatttatgtaaccatgactttgtaattttgagaattcctcccagtgatggtcagtattcttttggaatgtaaaccgatttaatgccaaaccaccttaacctttgtttctcagtgttccttaacagcctgccttttattaatctcaggcttttttatgaacactctcatttcagtagaatttggaaaactaagcgtggttggaatttctttgaattctgttagtaatgcccaaaagaaaagtctcaagcagtccccctatccagtcatttttatggagtttcatgttgtccactatagctggacactgaaccttttgcctaatttattataaaggcctgaccctctattgtcccatcttcacccccattccagagcagaggagtctctgtggaccatgaattgcactgtctccctcctcatttctaaatgaaaggtattagatataaatttttttgaaaggttagttgtttgagatgctaagcaggataataaatttagattttaaaatgttccctgtaaaagtcagcccatgacaaggaaatttacaaaatactagagtatctagaagggtgaaaacaaaaaaaaataaaaagaaacacagacgcccaggtgtcagctctccgtttaaagaatgaaaaatgtaactcatgatgatctgtgaaaccttcaaactaggaccaattgacttacttgatattctgcctttgatatggtagtacccacccggtattcctaaaatcctaaaaagatacaccttgcagtagcagaggcaatgacatgagtttgttttctcattaatatgaccagtttgggtctatgttggttcacatgtacatctactttatatgaaagaaaaaacagttgtctgcctgtaaaatgttgagtttcgattgagccatgtttggagattttattactattctgaagggtagtgttgttggttttcatcttcaagaagttgattccaaaactgagttatgaagaatgatataacagttccttcaaaattggcctaggaaataaaaccttaaaaggacaaaaaaaaaaa

SEQ ID NO: 7 - DNA, 2330 bp, artificial sequence
  Homo sapiens motile sperm domain containing 2 (MOSPD2), transcript variant 3
accgcctccccctcccacccttctctgtctacctctgggcgggactgccgggtgatgagatactcggtcggcgacggtagaacgggcgacggcgacaaccgcaatcacatccacgacggtgatcatggcagagaatcacgcccagaataaagccaagctcatctctgagacccggaggaggttcgaagctgagtatgtgacagataagtcagataaatatgatgcacgtgatgttgaaaggctacaacaagatgataactgggttgaaagttacttatcttggagacataatattgtagatgaaacactgaagatgctcgatgagagttttcagtggaggaaagaaatttctgtcaatgaccttaatgaatcctccattcccagatggttattggaaattggtgttatttatctccatggttatgacaaagaaggtaacaaattgttctggatcagggtgaagtatcatgtaaaagaccagaaaaccatattggacaaaaagaagctcatagcattctggttggaacgttatgctaagagggaaaatgggaaacctgtaacagtgatgtttgacctgtcagaaactggaataaatagcattgacatggactttgtacgctttatcatcaactgctttaaggtttattaccctaaatacctctcaaaaatagtgatctttgatatgccttggttaatgaatgctgctttcaaaattgtgaaaacctggcttggtccagaagcagtgagcttgttgaagtttacaagcaaaaatgaagtccaggactatgtcagtgtagaatacctgcctccccacatgggtggaactgatcctttcaagtatagctatccaccactagtagatgatgacttccagaccccactgtgtgagaatgggcctattaccagtgaggatgaaacttcaagtaaagaagacatagaaagtgatggcaaagaaacattggaaacaatttctaatgaagaacaaacacctcttcttaaaaagattaacccaaccgaatctacttccaaagcagaagaaaatgaaaaagttgattcaaaagtgaaagctttcaagaaaccattgagtgtatttaaaggccccttactacacatcagcccagcagaagaactgtactttggaagtacagaatccggagagaagaaaaccttaatagtgttgacaaatgtaactaaaaatatagtggcatttaaggtgagaacaacagctccagaaaaatacagagtcaagccaagcaatagcagctgtgacccgggtgcatcagtggatatagttgtgtctccccatgggggtttaacagtctctgcccaagaccgttttctgataatggctgcagaaatggaacagtcatctggcacaggcccagcagaattaactcagttttggaaagaagttcccagaaacaaagtgatggaacataggttaagatgccatactgttgaaagcagtaaaccaaacactcttacgttaaaagacaatgctttcaatatgtcagataaaaccagtgaagatatatgtctacaatttgccacctccagctgtgaaatggactgcagtccaccctaagtactgtgcacagtatctccctgtgtgtgtgcacagtggcttccccttacatggtagatttttggccttaatataatctaatcccaaagtagttgtgtatgttttctgttccttggcaaataaatgaagaaataattagccaagattgaaaatgtattgtcctaacggtgtccctttaatgtttcatatgaaaaattatgttgacccactaaaatatccttgctcaatgtctggtcagttgaatttaataacatatcttgttaatgtttgtgtgtctattaaatgtgactaagcaggattactgaaaattcactataaaatcaaaggcatctaaacgtttgtacttgtcttgattaatcatatatttacacttgatttttttctgtcttcatttgtttttatttaatcataattgcatgatttttttggtactctaatcagtaattttatttttaatcatgtcattacctattcatgaccaaattaccaaggaaccaacatttagatttagatatttgttttcacttaggaatggaaattaatagattttccatgaaagcattagtgaaatatcattaccttgatctgcaagtagcctaaaaatgcgattgctggtaaacctggcctcaaatttcatactaccataactgtttttatatattgccactaattttgactggatttaatagcactttattgtacaactacaaaaaaaaatatattcctagaattgttgccagtgtaa

SEQ ID NO: 8 - DNA, 3909 bp, Artificial Sequence
  Homo sapiens motile sperm domain containing 2 (MOSPD2), transcript variant X2
accgcctccccctcccacccttctctgtctacctctgggcgggactgccgggtgatgagatactcggtcggcgacggtagaacgggcgacggcgacaaccgcaatcacatccacgacggtgatcatggcagagaatcacgcccagaataaagccaagctcatctctgagacccggaggaggttcgaagctgagtatgtgacagataagtcagataaatatgatgcacgtgatgttgaaaggctacaacaagatgataactgggttgaaagttacttatcttggagacataatattgtagatgaaacactgaagatgctcgatgagagttttcagtggaggaaagaaatttctgtcaatgaccttaatgaatcctccattcccagatggttattggaaattggtgttatttatctccatggttatgacaaagaaggtaacaaattgttctggatcagggtgaagtatcatgtaaaagaccagaaaaccatattggacaaaaagaagctcatagcattctggttggaacgttatgctaagagggaaaatgggaaacctgtaacagtgatgtttgacctgtcagaaactggaataaatagcattgacatggactttgtacgctttatcatcaactgctttaaggtttattaccctaaatacctctcaaaaatagtgatctttgatatgccttggttaatgaatgctgctttcaaaattgtgaaaacctggcttggtccagaagcagtgagcttgttgaagtttacaagcaaaaatgaagtccaggactatgtcagtgtagaatacctgcctccccacatgggtggaactgatcctttcaagtatagctatccaccactagtagatgatgacttccagaccccactgtgtgagaatgggcctattaccagtgaggatgaaacttcaagtaaagaagacatagaaagtgatggcaaagaaacattggaaacaatttctaatgaagaacaaacacctcttcttaaaaagattaacccaaccgaatctacttccaaagcagaagaaaatgaaaaagttgattcaaaagtgaaagctttcaagaaaccattgagtgtatttaaaggccccttactacacatcagcccagcagaagaactgtactttggaagtacagaatccggagagaagaaaaccttaatagtgttgacaaatgtaactaaaaatatagtggcatttaaggtgagaacaacagctccagaaaaatacagagtcaagccaagcaatagcagctgtgacccgggtgcatcagtggatatagttgtgtctccccatgggggtttaacagtctctgcccaagaccgttttctgataatggctgcagaaatggaacagtcatctggcacaggcccagcagaattaactcagttttggaaagaagttcccagaaacaaagtgatggaacataggttaagatgccatactgttgaaagcagtaaaccaaacactcttacgttaaaagacaatgctttcaatatgtcagataaaaccagtgaagatatatgtctacaatacagttaaagaagtggtgccgggtaggaaccacggttccttcgtccattagttggaaaaagtaacagacctaaaactctaccaagctactaaaaacattgcacatctgtgcttcctaaaaggaaatatgcagcacgtggaggggaacacatacatgtcttgaaaataaactgctagaataaagaaatgctggagaaattgattataagagactatagctatttagtaaagtaagtaaaggcatatccattgtgtaaattaatagtttaaatataatttattttttccttttgatctgaatacttttaaagcttaagttttatcgtgtaaatacattagctaaactgaaaagtataagtaacatgctttgttgcagccaaaaaatgtaatctgcttttttatgacagaattattatagctgagctgacttactagcttttctatactatgtatatagaagaacatgtatattgagaaagaaaacatacttatatagaggaatttatgtaaccatgactttgtaattttgagaattcctcccagtgatggtcagtattcttttggaatgtaaaccgatttaatgccaaaccaccttaacctttgtttctcagtgttccttaacagcctgccttttattaatctcaggcttttttatgaacactctcatttcagtagaatttggaaaactaagcgtggttggaatttctttgaattctgttagtaatgcccaaaagaaaagtctcaagcagtccccctatccagtcatttttatggagtttcatgttgtccactatagctggacactgaaccttttgcctaatttattataaaggcctgaccctctattgtcccatcttcacccccattccagagcagaggagtctctgtggaccatgaattgcactgtctccctcctcatttctaaatgaaaggtattagatataaatttttttgaaaggttagttgtttgagatgctaagcaggataataaatttagattttaaaatgttccctgtaaaagtcagcccatgacaaggaaatttacaaaatactagagtatctagaagggtgaaaacaaaaaaaaataaaaagaaacacagacgcccaggtgtcagctctccgtttaaagaatgaaaaatgtaactcatgatgatctgtgaaaccttcaaactaggaccaattgacttacttgatattctgcctttgatatggtagtacccacccggtattcctaaaatcctaaaaagatacaccttgcagtagcagaggcaatgacatgagtttgttttctcattaatatgaccagtttgggtctatgttggttcacatgtacatctactttatatgaaagaaaaaacagttgtctgcctgtaaaatgttgagtttcgattgagccatgtttggagattttattactattctgaagggtagtgttgttggttttcatcttcaagaagttgattccaaaactgagttatgaagaatgatataacagttccttcaaaattggcctaggaaataaaaccttaaaaggacactggtgtgctactttgtcttaatttgggcttttctgtttcagtttgccacctccagctgtgaaatggactgcagtccaccctaagtactgtgcacagtatctccctgtgtgtgtgcacagtggcttccccttacatggtagatttttggccttaatataatctaatcccaaagtagttgtgtatgttttctgttccttggcaaataaatgaagaaataattagccaagattgaaaatgtattgtcctaacggtgtccctttaatgtttcatatgaaaaattatgttgacccactaaaatatccttgctcaatgtctggtcagttgaatttaataacatatcttgttaatgtttgtgtgtctattaaatgtgactaagcaggattactgaaaattcactataaaatcaaaggcatctaaacgtttgtacttgtcttgattaatcatatatttacacttgatttttttctgtcttcatttgtttttatttaatcataattgcatgatttttttggtactctaatcagtaattttatttttaatcatgtcattacctattcatgaccaaattaccaaggaaccaacatttagatttagatatttgttttcacttaggaatggaaattaatagattttccatgaaagcattagtgaaatatcattaccttgatctgcaagtagcctaaaaatgcgattgctggtaaacctggcctcaaatttcatactaccataactgtttttatatattgccactaattttgactggatttaatagcactttattgtacaactacaaaaaaaaatatattcctagaattgttgccagtgtaa

SEQ ID NO: 9 - protein, 769 aa, Artificial Sequence
  Integrin beta-2 isoform 1 precursor [Homo sapiens]
MLGLRPPLLALVGLLSLGCVLSQECTKFKVSSCRECIESGPGCTWCQKLNFTGPGDPDSIRCDTRPQLLMRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVHLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 10 - protein, 700 aa, Artificial Sequence
  Integrin beta-2 isoform 2 [Homo sapiens]
MRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVHLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 11 - protein, 700 aa, Artificial Sequence; identical to SEQ ID NO: 10
  Integrin beta-2 isoform X1 [Homo sapiens]

SEQ ID NO: 12 - protein, 238 aa, Artificial Sequence
  CD63, partial, Homo sapiens
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCIDVTVGCGINFNEKAIHKEGCVEKIGGWLRKNVLVVAAAALGIAFVEVLGIVFACCLVKSIRSGYEVM

(19) United States
(12) Patent Application Publication
(10) Pub. No.: US 2025/0277052 A1
(43) Pub. Date: Sep. 4, 2025

(54) MOTILE SPERM DOMAIN CONTAINING PROTEIN 2, INTEGRIN BETA2 AND CD63

(71) Applicant: IMMUNEWALK THERAPEUTICS, INC., Pearl River, NY (US)

(72) Inventors: ltzhak MENDEL, Rehovot (IL); Oshrat Propheta MEIRAN, Petach Tikva (IL); Yaniv SALEM, Kiryat Ono (IL); Pinhas KAFRI, Petach Tikva (IL); Niva YACOV, Tel Aviv (IL); Eyal BREITBART, Hashmona'im (IL)

(21) Appl. No.: 18/552,952

(22) PCT Filed: Mar. 28, 2022
(86) PCT No.: PCT/IB2022/052854

§ 371 Date: Sep. 28, 2023

Related U.S. Application Data
(60) Provisional application No. 63/167,325, filed on Mar. 29, 2021.

(51) Int. Cl. C07K 16/28; A61P 29/00; A61P 35/04

(52) U.S. Cl. CPC C07K 16/2896; A61P 29/00; A61P 35/04; C07K 16/2845

(57) ABSTRACT

The present disclosure relates to agents capable of modulating the interaction of Motile Sperm Domain Containing Protein 2 (MOSPD2) and CD63, MOSPD2 and integrin β2, CD63 and integrin β2, or a complex of MOSPD2, CD63 and integrin β2, compositions comprising the same, and methods of using the same. In addition, the present disclosure relates to compositions comprising an inhibitor of MOSPD2, an inhibitor of integrin β2, and/or an inhibitor of CD63, and methods of using the same.

### CROSS-REFERENCE TO RELATED APPLICATIONS

This application is the U.S. national phase of Int'l Application No. PCT/IB2022/052854, filed Mar. 28, 2022, which claims the benefit of U.S. Provisional Application No. 63/167,325, filed Mar. 29, 2021, which are hereby incorporated by reference herein in their entireties.

### REFERENCE TO A SEQUENCE LISTING SUBMITTED ELECTRONICALLY

The content of the electronically submitted sequence listing (Name: 5374_0970001_SequenceListing_ST25.txt; Size: 56,003 bytes; and Date of Creation: Apr. 19, 2024) is herein incorporated by reference in its entirety.

### FIELD

The present disclosure relates to agents capable of modulating the interaction of Motile Sperm Domain Containing Protein 2 (MOSPD2) and CD63, MOSPD2 and integrin β2, CD63 and integrin β2, or a complex of MOSPD2, CD63 and integrin β2, compositions comprising the same, and methods of using the same. In addition, the present disclosure relates to compositions comprising an inhibitor of MOSPD2, an inhibitor of integrin β2, and/or an inhibitor of CD63, and methods of using the same.

### BACKGROUND

Leukocytes are immune system cells involved in defending the body against infectious disease and foreign materials. Monocytes are a type of leukocytes and have critical roles in innate and adaptive immunity, immune surveillance, and particle scavenging. While a subset of monocytes is “resident” and recruited to tissues independently of inflammatory stimuli to assist in steady-state surveillance, wound-healing and resolution of inflammation, the majority (80-90%) of human circulating monocytes are classified as “inflammatory.” Circulating monocytes can sense inflammatory stimuli and quickly migrate through the vascular or lymphatic endothelium to the periphery, where they can differentiate into macrophages and dendritic cells (DCs) which cooperate with additional cell subsets to promote inflammation. While playing a necessary role in host defense, monocytes are nonetheless identified as critical mediators of inflammatory diseases and disorders.

Chemokine receptors and adhesion molecules play a key role in regulation of leukocyte trafficking. A complex array of chemokine receptors and G-protein coupled receptors (GPCRs) that are differentially expressed on leukocyte lineages and subsets, regulates which cell types migrate and to which tissue, under different conditions. Chemokines or chemotactic cytokines are secreted proteins that regulate migration and activation of leukocytes and stromal cells. Binding of chemokines to chemokine receptors activates signaling pathways such as the MAPK/ERK and PI3K/AKT pathways, resulting in phosphorylation of ERK and AKT, respectively. In the case of inflammatory monocytes, exit from the bone marrow across a monolayer of endothelial cells (diapedesis) to enter the circulatory system (intravasation) and to migrate to the inflamed tissue is dependent on C—C motif receptor 2 (CCR2) signaling, in response to activation by chemokine C—C motif ligand (CCL) 2 (also known as monocyte chemotactic protein-1; MCP-1) and CCL7 (MCP-3). On the other hand, constitutive migration of resident monocytes to non-inflamed tissues is mostly dependent on CCL3 (also known as Macrophage Inflammatory Protein-1α; MIP-1α) and chemokine (C—X3-C motif) ligand 1 (CX3CL1).

Inhibition of inflammatory cell migration (e.g., leukocyte chemotaxis) towards inflammatory sites is an attractive anti-inflammatory approach to treat chronic diseases. Suppressing the accumulation of unwanted monocytes and/or macrophages in chronically inflamed tissue has therapeutic potential, and migration inhibitors have accordingly demonstrated beneficial therapeutic results in animal models and clinical trials. Nevertheless, there have been several phase II clinical trial failures with chemokine and chemokine receptor antagonists, possibly due to redundancy of the target receptor and the complexity of heterogeneous diseases such as multiple sclerosis and rheumatoid arthritis.

Fibrosis is the formation of excess fibrous connective tissue in an organ or tissue. Fibrosis is similar to the process of scarring, in that both involve stimulated cells (e.g., fibroblasts) laying down connective tissue, including collagen and glycosaminoglycans. Fibrogenesis is a dynamic process and occurs in four phases: (i) initiation, due to injury of the organ/tissue; (ii) inflammation and activation of effector cells; (iii) enhanced synthesis of extracellular matrix (ECM); and (iv) deposition of ECM with progression to end-organ failure.

Fibrosis can cause severe morbidity and deleterious effects on patients' daily function, activity of daily living (ADL) and quality of life, and can lead to poor prognosis. For example, idiopathic pulmonary fibrosis (IPF) is a chronic intractable disease associated with worsening and debilitating shortness of breath. IPF patients become oxygen dependent, and have an average median survival time of three years and a five-year survival rate of 20% to 40% after diagnosis.

Metastasis, the spread of cancer cells from their tissue of origin to other organs, is a result of a multi-step process that involves several molecules. Evidence suggests that chemokines and chemokine receptors play an important role in tumor metastasis.

Motile Sperm Domain Containing Protein 2 (MOSPD2) is a 518-amino acid long, highly conserved protein with 90% homology between human and mouse. Bioinformatic analysis indicates that MOSPD2 contains a CRAL-TRIO region, named after the cellular retinaldehyde-binding protein (CRALBP) and the TRIO protein. MOSPD2 also contains a structurally related region to the nematode major sperm protein and one transmembrane region.

MOSPD2 is expressed on the surface of monocytes that have infiltrated into inflamed tissues and on several different tumor types (Int'l Pub. No. WO 2017/021857). MOSPD2 is associated with metastasis of cancer cells and promotes monocyte migration (Int'l Pub. No. WO 2017/021857). Accordingly, inhibition of MOSPD2 (e.g., with an anti-MOSPD2 antibody) has been described as a treatment for inflammatory diseases and disorders (Int'l Pub. No. WO 2017/021855) and for cancer and cancer metastasis (Int'l Pub. No. WO 2017/021857).

Integrins are integral cell-surface proteins composed of an alpha chain and a beta chain, and are crucial for cells to be able to efficiently bind to the extracellular matrix (ECM). This is especially important for neutrophils, as cellular adhesion plays a large role in extravasation from the blood vessels. A given alpha or beta chain may combine with multiple partners, resulting in different integrins. Leukocyte migration from the periphery to sites of inflammation is a multistage, tightly-regulated process in which integrins play a key role. Integrins mediate leukocyte adhesion to endothelial cells of blood vessels and are involved in leukocyte travel through the ECM. Integrins are transmembrane surface expressed heterodimers comprised of a/P subunits.

Integrin β2 (also known as integrin beta2, integrin β2, beta2 integrin, β2 integrin, or CD18) is an integrin beta chain protein that is encoded by the ITGB2 gene in humans. Upon binding with one of several different alpha chains, integrin β2 is capable of forming multiple heterodimers, which play significant roles in cellular adhesion and cell surface signaling, as well as important roles in immune responses. In particular, integrin β2 is involved in adhesion to endothelial cells and ECM. Laboratory Investigation (2002), 82: 521-534. Integrin β2 also exists in soluble, ligand binding forms. Deficiencies in integrin β2 expression can lead to adhesion defects in circulating white blood cells in humans, reducing the immune system's ability to fight off foreign invaders.

The integrin β2 subgroup of integrins includes αLβ2, αMβ2, αXβ2 and αDβ2 heterodimers. Integrin β2 can bind a myriad of ligands, including the intercellular adhesion molecule (ICAM) and vascular cell adhesion molecule (VCAM) family of adhesion molecules upregulated on activated endothelial cells, and components of the ECM like fibronectin, collagen, and heparin.

Structural and signaling studies have shown that integrins can adopt three conformational states: bent-closed, extended-close, and extended-open. Inactive integrins display a bent-closed conformation which precludes engagement with counterpart ligands. Upon ligation of chemokine receptors or toll like receptors, an inside-out signaling induces a transition to the intermediate extended-close and further on to the high affinity extended-open conformation. Binding of the high affinity form to extracellular ligands leads to integrin clustering and strengthened integrin-ligand adhesion. Regulation of integrins is therefore an important piece in controlling leukocyte migration and inflammation.

CD63 (also known as LAMP-3, ME491, MLA1, OMA81H, or TSPAN30) is a protein mainly associated with membranes of intracellular vesicles, although cell surface expression may be induced. CD63 is a member of the transmembrane 4 superfamily, also known as the tetraspanin family. It is a cell-surface protein that is characterized by the presence of four hydrophobic domains and is known to complex with integrins. In particular, CD63 interacts with integrins β1 and β2 to promote cell adhesion to the extracellular matrix. J. Exp. Med. (2005), 201: 385-396. CD63 is expressed by human primary monocytes and neutrophils. CD63 also mediates signal transduction events that play a role in the regulation of cell development, activation, growth, and motility, and is associated with tumor progression. Deficiency of CD63 is associated with Hermansky-Pudlak Syndrome.

There is a need for additional treatments for inflammatory diseases and disorders and treatments for cancer and metastasis of cancer directed to new combinations of targets.

### BRIEF SUMMARY

Provided herein is a method of inhibiting migration of an inflammatory cell, comprising contacting the inflammatory cell with an isolated agent capable of modulating the interaction of motile sperm domain containing protein 2 (MOSPD2) and CD63.

Also provided herein is a method of promoting adhesion of an inflammatory cell to an extracellular matrix component, comprising contacting the inflammatory cell with an isolated agent capable of modulating the interaction of MOSPD2 and CD63.

Also provided herein is a method of inhibiting migration of an inflammatory cell, comprising contacting the inflammatory cell with an isolated agent capable of modulating the interaction of MOSPD2 and integrin β2.

Also provided herein is a method of promoting adhesion of an inflammatory cell to an extracellular matrix component, comprising contacting the inflammatory cell with an isolated agent capable of modulating the interaction of MOSPD2 and integrin β2.

Also provided herein is a method of modulating adhesion of an inflammatory cell to an extracellular matrix component, comprising contacting the inflammatory cell with an isolated agent capable of modulating the interaction of MOSPD2 and integrin β2.

Also provided herein is a method of inhibiting migration of an inflammatory cell, comprising contacting the inflammatory cell with an isolated agent capable of modulating the interaction of CD63 and integrin β2.

Also provided herein is a method of promoting adhesion of an inflammatory cell to an extracellular matrix component, comprising contacting the inflammatory cell with an isolated agent capable of modulating the interaction of CD63 and integrin β2.

Also provided herein is a method of modulating adhesion of an inflammatory cell to an extracellular matrix component, comprising contacting the inflammatory cell with an isolated agent capable of modulating the interaction of CD63 and integrin β2.

Also provided herein is a method of inhibiting migration of an inflammatory cell, comprising contacting the inflammatory cell with an isolated agent capable of modulating the interaction of a complex of MOSPD2, integrin β2, and CD63.

Also provided herein is a method of promoting adhesion of an inflammatory cell to an extracellular matrix component, comprising contacting the inflammatory cell with an isolated agent capable of modulating the interaction of a complex of MOSPD2, integrin β2, and CD63.

Also provided herein is a method of modulating adhesion of an inflammatory cell to an extracellular matrix component, comprising contacting the inflammatory cell with an isolated agent capable of modulating the interaction of a complex of MOSPD2, integrin β2, and CD63.

Also provided herein is a method of promoting adhesion of an inflammatory cell to an adhesion molecule, comprising contacting the inflammatory cell with an isolated agent capable of modulating the interaction of MOSPD2 and CD63.

Also provided herein is a method of promoting adhesion of an inflammatory cell to an adhesion molecule, comprising contacting the inflammatory cell with an isolated agent capable of modulating the interaction of MOSPD2 and integrin β2.

Also provided herein is a method of modulating adhesion of an inflammatory cell to an adhesion molecule, comprising contacting the inflammatory cell with an isolated agent capable of modulating the interaction of MOSPD2 and integrin β2.

Also provided herein is a method of promoting adhesion of an inflammatory cell to an adhesion molecule, comprising contacting the inflammatory cell with an isolated agent capable of modulating the interaction of CD63 and integrin β2.

Also provided herein is a method of modulating adhesion of an inflammatory cell to an adhesion molecule, comprising contacting the inflammatory cell with an isolated agent capable of modulating the interaction of CD63 and integrin β2.

Also provided herein is a method of promoting adhesion of an inflammatory cell to an adhesion molecule, comprising contacting the inflammatory cell with an isolated agent capable of modulating the interaction of a complex of MOSPD2, integrin β2, and CD63.

Also provided herein is a method of modulating adhesion of an inflammatory cell to an adhesion molecule, comprising contacting the inflammatory cell with an isolated agent capable of modulating the interaction of a complex of MOSPD2, integrin β2, and CD63.

Also provided herein is a method of inducing expression of a protein associated with integrin activation in an inflammatory cell, comprising contacting the inflammatory cell with an isolated agent capable of modulating the interaction of MOSPD2 and CD63.

Also provided herein is a method of inducing expression of a protein associated with integrin activation in an inflammatory cell, comprising contacting the inflammatory cell with an isolated agent capable of modulating the interaction of MOSPD2 and integrin 02.

Also provided herein is a method of inducing expression of a protein associated with integrin activation in an inflammatory cell, comprising contacting the inflammatory cell with an isolated agent capable of modulating the interaction of CD63 and integrin β2.

Also provided herein is a method of inducing expression of a protein associated with integrin activation in an inflammatory cell, comprising contacting the inflammatory cell with an isolated agent capable of modulating the interaction of a complex of MOSPD2, integrin β2, and CD63.

Also provided herein is a method of treating or preventing an inflammatory disease or disorder, comprising administering a therapeutically effective amount of an isolated agent capable of modulating the interaction of MOSPD2 and CD63 to a subject in need thereof.

Also provided herein is a method of treating or preventing an inflammatory disease or disorder, comprising administering a therapeutically effective amount of an isolated agent capable of modulating the interaction of MOSPD2 and integrin β2 to a subject in need thereof.

Also provided herein is a method of treating or preventing an inflammatory disease or disorder, comprising administering a therapeutically effective amount of an isolated agent capable of modulating the interaction of CD63 and integrin β2 to a subject in need thereof.

Also provided herein is a method of treating or preventing an inflammatory disease or disorder, comprising administering a therapeutically effective amount of agent capable of modulating the interaction of a complex of MOSPD2, integrin β2, and CD63 to a subject in need thereof.

Also provided herein is a method of treating, preventing or reducing the incidence of cancer metastasis, comprising administration of a therapeutically effective amount of an agent capable of modulating the interaction of MOSPD2 and CD63 to a subject in need thereof.

Also provided herein is a method of treating, preventing or reducing the incidence of cancer metastasis, comprising administration of a therapeutically effective amount of an agent capable of modulating the interaction of MOSPD2 and integrin β2 to a subject in need thereof.

Also provided herein is a method of treating, preventing or reducing the incidence of cancer metastasis, comprising administration of a therapeutically effective amount of an agent capable of modulating the interaction of CD63 and integrin β2 to a subject in need thereof.

Also provided herein is a method of treating, preventing or reducing the incidence of cancer metastasis, comprising administration of a therapeutically effective amount of an agent capable of modulating the interaction of a complex of MOSPD2, integrin β2, and CD63 to a subject in need thereof.

Also provided herein is an isolated agent capable of modulating the interaction of MOSPD2 and CD63.

Also provided herein is an isolated agent capable of modulating the interaction of MOSPD2 and integrin β2.

Also provided herein is an isolated agent capable of modulating the interaction of CD63 and integrin β2.

Also provided herein is an isolated agent capable of modulating the interaction of a complex of MOSPD2, integrin β2, and CD63.

Also provided herein is a composition comprising (i) an inhibitor of MOSPD2, and (ii) an inhibitor of integrin β2 or an inhibitor of CD63.

Also provided herein is a composition comprising (i) an inhibitor of integrin β2, and (ii) an inhibitor of CD63.

Also provided herein is a composition comprising (i) an inhibitor of MOSPD2, (ii) an inhibitor of integrin β2, and (iii) an inhibitor of CD63.

Also provided herein is a method of treating or preventing an inflammatory disease or disorder, comprising administering (i) a therapeutically effective amount of an inhibitor of MOSPD2, and (ii) a therapeutically effective amount of an inhibitor of integrin β2 or a therapeutically effective amount of an inhibitor of CD63 to a subject in need thereof.

Also provided herein is a method of treating or preventing an inflammatory disease or disorder, comprising administering (i) a therapeutically effective amount of an inhibitor of integrin β2, and (ii) a therapeutically effective amount of an inhibitor of CD63 to a subject in need thereof.

Also provided herein is a method of treating or preventing an inflammatory disease or disorder, comprising administering (i) a therapeutically effective amount of an inhibitor of MOSPD2, (ii) a therapeutically effective amount of an inhibitor of integrin 02, and (iii) a therapeutically effective amount of an inhibitor of CD63 to a subject in need thereof.

Also provided herein is a method of treating, preventing or reducing the incidence of cancer metastasis comprising administering (i) a therapeutically effective amount of an inhibitor of MOSPD2, and (ii) a therapeutically effective amount of an inhibitor of integrin β2 or a therapeutically effective amount of an inhibitor of CD63 to a subject in need thereof.

Also provided herein is a method of treating, preventing or reducing the incidence of cancer metastasis comprising administering (i) a therapeutically effective amount of an inhibitor of integrin β2, and (ii) a therapeutically effective amount of an inhibitor of CD63 to a subject in need thereof.

Also provided herein is a method of treating, preventing or reducing the incidence of cancer metastasis comprising administering (i) a therapeutically effective amount of an inhibitor of MOSPD2, (ii) a therapeutically effective amount of an inhibitor of integrin 02, and (iii) a therapeutically effective amount of an inhibitor of CD63 to a subject in need thereof.

### BRIEF DESCRIPTION OF THE DRAWINGS

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1A shows the number of neutrophils counted at 1, 2 and 4 hours after the induction of inflammation with thioglycolate in wild type mice and CD63 knockout (CD63−/−) mice. FIG. 1A is reproduced from Blood 2011, 118:4265-4273.

FIG. 1B shows the number of macrophages counted 3 days after the induction of inflammation with thioglycolate in wild type (W.T) mice and MOSPD2 knock out (MOSPD2-KO) mice. ***p<0.001 by t-test.

FIG. 2A shows staining of THP-1 cells having CD63 silenced by CRISPR (THP-1 CD63-CRISPR) and control-silenced THP-1 cells (THP-1 control-CRISPR-IgG and THP-1 control-CRISPR) detected by flow cytometry.

FIG. 2B shows migration of CD63-silenced THP-1 cells (CD63-CRISPR) relative to control THP-1 cells (Control-CRISPR) towards SDF-1 and MCP-1 (100 ng/ml each). Samples were run in triplicate. Results are shown as mean±SD. ***p<0.001 by t-test.

FIG. 3A shows protein expression of MOSPD2 in THP-1 cells having MOSPD2 silenced by CRISPR (transduced with CRISPR-MOSPD2 lentiviral particles) or control-silenced THP-1 cells (Control). Two different isolated clones (MOSPD2 M1 and MOSPD2 M2) are shown for CRISPR-MOSPD2 cells. HSP-90 was used as a loading control.

FIG. 3B shows transwell migration of CRISPR-Control and CRISPR-MOSPD2 cells described in FIG. 3A toward RANTES and MCP-1 (100 ng/ml each). Cell count is presented. Samples were run in triplicate. Results are shown as mean±SD. **p<0.01 by t-test.

FIG. 3C shows adhesion of CRISPR-Control and CRISPR-MOSPD2 cells described in FIG. 3A on a plate coated with Collagen IV. The percentage of adherent CRISPR-MOSPD2 cells relative to adherent CRISPR-Control cells is presented. Samples were run in triplicate. Results are shown as mean±SD. **p<0.01, ***p<0.001 by t-test.

FIG. 3D shows adhesion of CRISPR-Control and CRISPR-MOSPD2 M1 cells described in FIG. 3A to different extracellular matrix (ECM) proteins. The percentage of adherent CRISPR-MOSPD2 cells relative to adherent CRISPR-Control cells is presented. Samples were run in triplicate. Results are shown as mean±SD. *p<0.05, **p<0.01, ***p<0.001, n.s.=not statistically significant by t-test.

FIG. 4A shows staining of THP-1 cells having integrin β2 silenced by CRISPR (transduced with CRISPR-Integrin β2 lentiviral particles) and control-silenced THP-1 cells (Control) detected by flow cytometry. Two different isolated clones (Integrin β2 I1 and Integrin β2 I2) are shown for CRISPR-Integrin β2 cells. CRISPR-Control cells were also stained with a phycoerythrin (PE)-labeled isotype-matched antibody.

FIG. 4B shows a comparison of transwell migration between CRISPR-Control and CRISPR-MOSPD2 cells described in FIG. 3A and CRISPR-Integrin β2 cells described in FIG. 4A toward RANTES and MCP-1 (100 ng/mL each). Cell count is presented. Samples were run in triplicate. Results are shown as mean±SD. *p<0.05 by t-test.

FIG. 4C shows adhesion of CRISPR-Control and CRISPR-Integrin β2 I2 cells described in FIG. 4A to a plate coated with different ECM proteins. The percentage of adherent CRISPR-Integrin 62 2 I2 cells relative to adherent CRISPR-Control cells is presented. Samples were run in triplicate. Results are shown as mean±SD. *p<0.05, **p<0.01, ***p<0.001 by t-test.

FIG. 5A and FIG. 5B show western blot analysis of constitutively expressing HA-tagged MOSPD2-expressing HEK293 cells un-transfected or transiently transfected with FLAG-tagged CD63. After immunoprecipitation cell lysate samples were loaded on SDS-PAGE gel and blotted with anti-FLAG antibody (FIG. 5A) or anti-MOSPD2 antibody (FIG. 5B).

FIG. 6A and FIG. 6B show western blot analysis of constitutively expressing HA-tagged MOSPD2-expressing HEK293 cells un-transfected or transiently transfected with FLAG-tagged integrin β2. After immunoprecipitation cell lysate samples were loaded on SDS-PAGE gel and blotted with anti-FLAG antibody (FIG. 6A) or anti-MOSPD2 antibody (FIG. 6B).

FIG. 7A shows the percentage of migrating monocytes (CD14 primary cells) following treatment with 10 μg/mL of anti-MOSPD2 antibody (αMOSPD2), anti-integrin β2 mAb antibody (α-Intg β2), anti-CD63 antibody (αCD63), or control antibody (IgG). Statistical significance of p<0.001 is indicated (t-test).

FIG. 7B shows transwell migration of CD14+ monocytes treated with control or anti-integrin β2 polyclonal antibody (α-Intg β2) toward RANTES and MCP-1 (100 ng/mL each). Cell count is presented. Samples were run in triplicate. Results are shown as mean±SD.

FIG. 8 shows the percentage of adherent monocytes (CD14+ primary cells) to fibronectin following treatment with 10 μg/mL of anti-MOSPD2 antibody (αMOSPD2), anti-integrin β2 antibody mAb (α-Intg β2), anti-CD63 antibody (αCD63), or control antibody (IgG). Statistical significance of **p<0.01, ***p<0.001 is indicated (t-test).

FIG. 9 shows adhesion of CD14+ monocytes treated with control or anti-integrin β2 polyclonal antibody (α-Intg β2) to a plate coated with different ECM proteins. The percentage of adherent cells treated with α-Intg β2 relative to adherent control cells is presented. Samples were run in triplicate. Results are shown in mean±SD. *p<0.05 by t-test.

FIG. 10 shows the percentage of adherent monocytes (CD14+ primary cells) to fibronectin following treatment with 0.1, 1 or 10 μg/mL of anti-MOSPD2 antibody (αMOSPD2) or 10 μg/mL control antibody (IgG). Statistical significance of *p<0.05 is indicated (t-test).

FIG. 11 shows the percentage of adherent monocytes (CD14+ primary cells) to collagen IV following treatment with 10 μg/mL of anti-MOSPD2 antibody (αMOSPD2) or control antibody (IgG). Statistical significance of ***p<0.001 is indicated (t-test).

FIG. 12 shows adhesion of CD14+ monocytes treated with isotype control or anti-MOSPD2 antibody (αMOSPD2, 10 μg/mL) to a plate coated with different ECM proteins. The percentage of adherent cells treated with αMOSPD2 relative to adherent control cells is presented. Samples were run in triplicate. Results are shown in mean SD. *p<0.05, **p<0.01, ***p<0.001 by t-test.

FIG. 13 shows adhesion of CD14+ monocytes treated with isotype control or anti-MOSPD2 antibody (αMOSPD2, 10 μg/mL) to a plate coated with ICAM-1 or VCAM-1. The percentage of adherent cells treated with αMOSPD2 relative to adherent control cells is presented. Samples were run in triplicate. Results are shown in mean±SD. is shown. ***p<0.001 by t-test.

FIG. 14 shows representative western blot images from experiments to determine the presence of phosphorylated FAK (p-FAK), phosphorylated AKT (p-AKT), phosphorylated ERK1/2 (p-ERK1/2), or phosphorylated P38 (p-P38) at 5, 15 and 30 minutes following treatment with 10 μg/mL of anti-MOSPD2 antibody (αMOSPD2) or control antibody (IgG). C-YES was used as a loading control.

FIG. 15 shows the results from an experiment to determine the percentage of migrating monocytes (CD14 primary cells) following treatment with 10 μg/mL of anti-MOSPD2 antibody (αMOSPD2), Natalizumab (anti-α4β1 antibody), Vedolizumab (anti-α4β7 antibody), or control antibody (IgG). Statistical significance of ***p<0.001 is indicated (t-test).

FIG. 16A and FIG. 16B show flow cytometry analysis for expression of MOSPD2 on HEK293 cells sham transduced or stably transduced with HA-tagged MOSPD2 and stained with anti-HA-PE antibody (FIG. 16A), or CD63 on stably expressing MOSPD2 HEK293 cells that were sham transfected or transiently transfected with FLAG-tagged CD63 and stained with anti-CD63-PE (FIG. 16B).

FIG. 17 shows flow cytometry analysis for expression of integrin β2 on stably expressing HA-tagged MOSPD2 expressing HEK293 cells that were sham transfected or transiently transfected with FLAG-tagged integrin β2 and stained with anti-integrin β2-PE.

FIG. 18A and FIG. 18B show anti-MOSPD2 and anti-CD63 antibodies promote monocyte binding to endothelial cell adhesion molecules. Adhesion to ICAM-1 (FIG. 18A) and VCAM-1 (FIG. 18B) coated plates of freshly isolated human primary monocytes incubated with 10 μg/ml of the indicated antibodies. Adhesion relative to IgG control antibody is shown. Samples were run in triplicate. Results are shown as mean SD. Statistical significance of ***p<0.001 indicated (t-test).

FIG. 19A and FIG. 19B show anti-MOSPD2 antibody induces low to high affinity transition of LFA-1 on monocytes. Primary monocytes (FIG. 19A) or THP-1 monocytic cells (FIG. 19B) were incubated with 10 μg/ml of IgG control or anti-MOSPD2 antibody, stained with fluorophore conjugated anti-LFA-1 m24 monoclonal antibody (mAb), and analyzed by flow cytometry.

FIG. 20A shows staining of CRISPR-Control or CRISPR-MOSPD2 M1 and M2 clones described in FIG. 3A with anti-LFA-1 clone m24, as detected by flow cytometry. CRISPR-Control cells were also stained with an isotype-control antibody (IgG).

FIG. 20B shows staining of THP-1 cells treated with isotype control antibody (IgG, 10 g/mL) or different doses of anti-MOSPD2 antibody (αMOSPD2, 0.1 g/mL, 1 g/mL, or 10 g/mL) with anti-LFA-1 clone m24, as detected by flow cytometry.

FIG. 20C shows flow cytometry results of THP-1 cells treated with isotype control antibody or anti-MOSPD2 antibody (αMOSPD2, 10 μg/ml) for the indicated times and stained with anti-LFA-1 clone m24.

FIG. 20D shows flow cytometry results of CD14+ monocytes treated with isotype control antibody (IgG, 10 g/mL) or different doses of anti-MOSPD2 antibody (αMOSPD2, 1 g/mL or 10 g/mL) and stained with anti-LFA-1 clone m24.

FIGS. 20E and 20F show representative western blot images from experiments to determine the presence of phosphorylated FAK (p-FAK at amino acid residues Y407 and Y397), phosphorylated AKT (p-AKT) and phosphorylated ERK1/2 (p-ERK1/2) of THP-1 (FIG. 20E) or CD14+ monocytes (FIG. 20F) treated with isotype control antibody (IgG) or anti-MOSPD2 antibody (αMOSPD2, 10 μg/ml) at the indicated time points. HSP-90 and C-YES were used as loading controls for FIGS. 20E and 20F, respectively.

FIG. 21A shows tumor area (%) of anti-MOSPD2 antibody (Anti MOSPD2 mAb) treated mice versus control antibody treated mice (Control Ab). FIG. 21B shows representative pictures of lung hematoxylin and eosin (H&E) staining sections. Left side—Control Ab treated. Right side—Anti MOSPD2 mAb treated.

### DETAILED DESCRIPTION OF THE INVENTION

In order that the present disclosure may be more readily understood, certain terms are first defined. As used in this application, except as otherwise expressly provided herein, each of the following terms shall have the meaning set forth below. Additional definitions are set forth throughout the application.

### Definitions

Various terms relating to aspects of disclosure are used throughout the specification and claims. Such terms are to be given their ordinary meaning in the art, unless otherwise indicated. Other specifically defined terms are to be construed in a manner consistent with the definition provided herein.

As used herein, “MOSPD2” refers to any polypeptide classified as “Motile Sperm Domain Containing Protein 2” or “Motile Sperm Domain Containing 2.” Examples of MOSPD2 include, but are not limited to, the polypeptides of SEQ ID NOs:1-4, or any variant thereof (e.g., having a sequence at least 75%, at least 80%, at least 85%, at least 90%, at least 95%, at least 96%, at least 97%, at least 98%, or at least 99% identical to any one of SEQ ID NOs:1-4). Other examples of MOSPD2 include, but are not limited to, a polypeptide encoded by a polynucleotide of any one of SEQ ID NOs:5-8, or any variant thereof (e.g., a polynucleotide having at least 75%, at least 80%, at least 85%, at least 90%, at least 95%, at least 96%, at least 97%, at least 98%, or at least 99% identical to any one of SEQ ID NOs:5-8). Other examples of MOSPD2 can be identified by searching public databases (e.g., BLAST), as well known to one skilled in the art.

As used herein, “integrin β2” (also known as integrin beta2, integrin β2, integrin beta chain 2, or CD18) refers to an integrin beta chain protein that is encoded by the ITGB2 gene. Integrin β2 is involved in modulating adhesion of inflammatory cells to endothelial cells and/or extracellular matrix, and modulating adhesion of endothelial cells and/or extracellular matrix to inflammatory cells. Examples of integrin β2 include, but are not limited to, the polypeptides of SEQ ID NOs:9-11, or any variant thereof (e.g., having a sequence at least 75%, at least 80%, at least 85%, at least 90%, at least 95%, at least 96%, at least 97%, at least 98%, or at least 99% identical to any one of SEQ ID NOs:9-11). Other examples of integrin β2 can be identified by searching public databases (e.g., BLAST), as well known to one skilled in the art.

As used herein, “CD63” (also known as LAMP-3, ME491, MLA1, OMA81H, or TSPAN30) refers to a protein that is encoded by the CD63 gene. CD63 is a member of the transmembrane 4 superfamily, and a cell-surface protein characterized by the presence of four hydrophobic domains and its ability to complex with integrins. Examples of CD63 include, but are not limited to, the polypeptides of SEQ ID NO:12, or any variant thereof (e.g., having a sequence at least 75%, at least 80%, at least 85%, at least 90%, at least 95%, at least 96%, at least 97%, at least 98%, or at least 99% identical to SEQ ID NO:12). Other examples of CD63 can be identified by searching public databases (e.g., BLAST), as well known to one skilled in the art.

As used herein, “modulating” an interaction means reducing, blocking, inhibiting, increasing, promoting, or activating the interaction (or the like), either directly or indirectly.

As used herein, an “agent capable of modulating the interaction of motile sperm domain containing protein 2 (MOSPD2) and CD63” (or CD63 and MOSPD2) refers to any protein, DNA, or RNA compound that results in reducing, blocking, inhibiting, increasing, promoting, or activating, directly or indirectly, the interaction of MOSPD2 and CD63 (or CD63 and MOSPD2). The agent can be, for example, an anti-MOSPD2 antibody, anti-CD63 antibody, or antigen binding fragment thereof. The agent can also be, for example, an antisense DNA, decoy DNA, double-stranded DNA, single-stranded DNA, complexed DNA, encapsulated DNA, viral DNA, plasmid DNA, RNAi, miRNA, siRNA, shRNA, antisense RNA, naked RNA, encapsulated RNA, viral RNA, double-stranded RNA, a molecule capable of generating RNA interference, or a gene editing system directed against MOSPD2 and/or CD63 (e.g., directed against a gene or mRNA encoding MOSPD2 and/or CD63).

As used herein, an “agent capable of modulating the interaction of motile sperm domain containing protein 2 (MOSPD2) and integrin β2” (or integrin β2 and MOSPD2) refers to any protein, DNA, or RNA compound that results in reducing, blocking, inhibiting, increasing, promoting, or activating, directly or indirectly, the interaction of MOSPD2 and integrin β2 (or integrin β2 and MOSPD2). The agent can be, for example, an anti-MOSPD2 antibody, anti-integrin β2 antibody, or antigen binding fragment thereof. The agent can also be, for example, an antisense DNA, decoy DNA, double-stranded DNA, single-stranded DNA, complexed DNA, encapsulated DNA, viral DNA, plasmid DNA, RNAi, miRNA, siRNA, shRNA, antisense RNA, naked RNA, encapsulated RNA, viral RNA, double-stranded RNA, a molecule capable of generating RNA interference, or a gene editing system directed against MOSPD2 and/or integrin β2 (e.g., directed against a gene or mRNA encoding MOSPD2 and/or integrin β2).

As used herein, an “agent capable of modulating the interaction of CD63 and integrin β2” (or integrin β2 and CD63) refers to any protein, DNA, or RNA compound that results in reducing, blocking, inhibiting, increasing, promoting, or activating, directly or indirectly, the interaction of CD63 and integrin β2 (or integrin β2 and CD63). The agent can be, for example, an anti-CD63 antibody, anti-integrin β2 antibody, or antigen binding fragment thereof. The agent can also be, for example, an antisense DNA, decoy DNA, double-stranded DNA, single-stranded DNA, complexed DNA, encapsulated DNA, viral DNA, plasmid DNA, RNAi, miRNA, siRNA, shRNA, antisense RNA, naked RNA, encapsulated RNA, viral RNA, double-stranded RNA, a molecule capable of generating RNA interference, or a gene editing system directed against CD63 and/or integrin β2 (e.g., directed against a gene or mRNA encoding CD63 and/or integrin β2).

As used herein, an “agent capable of modulating the interaction of motile sperm domain containing protein 2 (MOSPD2), integrin β2 and CD63” refers to any protein, DNA, or RNA that results in reducing, blocking, inhibiting, increasing, promoting, or activating, directly or indirectly, the interaction of a complex of MOSPD2, integrin β2, and/or CD63. The agent can be, for example, an anti-MOSPD2 antibody, anti-integrin β2 antibody, anti-CD63 antibody, or antigen binding fragment thereof. The agent can also be, for example, an antisense DNA, decoy DNA, double-stranded DNA, single-stranded DNA, complexed DNA, encapsulated DNA, viral DNA, plasmid DNA, RNAi, miRNA, siRNA, shRNA, antisense RNA, naked RNA, encapsulated RNA, viral RNA, double-stranded RNA, a molecule capable of generating RNA interference, or a gene editing system directed against MOSPD2, integrin β2, and/or CD63 (e.g., directed against a gene or mRNA encoding MOSPD2, integrin β2, and/or CD63). The agent can, for example, inhibit or reduce the interaction by specifically binding to MOSPD2, integrin β2, or CD63, or two or more of MOSPD2, integrin β2, and CD63.

As used herein, a “gene editing system” refers to a type of genetic engineering in which DNA is inserted, deleted, modified or replaced in a genome of a living organism. CRISPR (clustered regularly interspaced short palindromic repeat) gene editing is one example of a gene editing system. In some aspects, the CRISPR system is a CRISPR/CAS system or CRISPR/CAS9 system.

As used herein, “an inhibitor of MOSPD2” and “a MOSPD2 inhibitor” refer to any compound that downregulates an activity of MOSPD2. The inhibitor can be, for example, a polypeptide, DNA, or RNA. The inhibitor can also be, for example, a molecule that specifically binds to a MOSPD2 polypeptide, a molecule that specifically binds to a ligand of a MOSPD2 polypeptide, an antisera raised against a MOSPD2 polypeptide, a soluble MOSPD2 polypeptide, or a soluble MOSPD2 polypeptide comprising, consisting essentially of, or consisting of an extracellular domain of a MOSPD2 polypeptide. The inhibitor can also be, for example, an antibody that specifically binds to a MOSPD2 polypeptide or an antigen binding fragment of an antibody that specifically binds to a MOSPD2 polypeptide. The inhibitor can also be, for example, an RNAi, miRNA, siRNA, shRNA, antisense RNA, antisense DNA, decoy molecule, decoy DNA, double-stranded DNA, single-stranded DNA, complexed DNA, encapsulated DNA, viral DNA, plasmid DNA, naked RNA, encapsulated RNA, viral RNA, double-stranded RNA, molecule capable of generating RNA interference, or combinations thereof, that hybridizes to a nucleotide sequence encoding a MOSPD2 polypeptide.

As used herein, “an inhibitor of CD63” and “a CD63 inhibitor” refer to any compound that downregulates an activity of CD63. The inhibitor can be, for example, a polypeptide, DNA, or RNA. The inhibitor can also be, for example, a molecule that specifically binds to a CD63 polypeptide, a molecule that specifically binds to a ligand of a CD63 polypeptide, or an antisera raised against a CD63 polypeptide. The inhibitor can also be, for example, an antibody that specifically binds to a CD63 polypeptide or an antigen binding fragment of an antibody that specifically binds to a CD63 polypeptide. The inhibitor can also be, for example, an RNAi, miRNA, siRNA, shRNA, antisense RNA, antisense DNA, decoy molecule, decoy DNA, double-stranded DNA, single-stranded DNA, complexed DNA, encapsulated DNA, viral DNA, plasmid DNA, naked RNA, encapsulated RNA, viral RNA, double-stranded RNA, molecule capable of generating RNA interference, or combinations thereof, that hybridizes to a nucleotide sequence encoding a CD63 polypeptide.

As used herein, “an inhibitor of integrin β2” and “a integrin β2 inhibitor” refer to any compound that downregulates an activity of integrin β2. The inhibitor can be, for example, a polypeptide, DNA, or RNA. The inhibitor can also be, for example, a molecule that specifically binds to an integrin β2 polypeptide, a molecule that specifically binds to a ligand of a integrin β2 polypeptide, or an antisera raised against a integrin β2 polypeptide. The inhibitor can also be, for example, an antibody that specifically binds to an integrin β2 polypeptide or an antigen binding fragment of an antibody that specifically binds to an integrin β2 polypeptide. The inhibitor can also be, for example, an RNAi, miRNA, siRNA, shRNA, antisense RNA, antisense DNA, decoy molecule, decoy DNA, double-stranded DNA, single-stranded DNA, complexed DNA, encapsulated DNA, viral DNA, plasmid DNA, naked RNA, encapsulated RNA, viral RNA, double-stranded RNA, molecule capable of generating RNA interference, or combinations thereof, that hybridizes to a nucleotide sequence encoding an integrin β2 polypeptide.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

An “antigen binding fragment” or “antigen binding fragment thereof” refer to a portion of an intact antibody that binds to an antigen. An antigen binding fragment can contain an antigen recognition site of an intact antibody (e.g., complementarity determining regions (CDRs) sufficient to specifically bind antigen). Examples of antigen binding fragments of antibodies include, but are not limited to Fab, Fab′, F(ab′)₂, and Fv fragments, linear antibodies, and single chain antibodies. An antigen binding fragment can be derived from any animal species, such as rodents (e.g., mouse, rat, or hamster) and humans or can be artificially produced.

A “monoclonal” antibody or antigen binding fragment thereof refers to a homogeneous antibody or antigen binding fragment population involved in the highly specific binding of a single antigenic determinant, or epitope. This is in contrast to polyclonal antibodies that typically include different antibodies directed against different antigenic determinants. The term “monoclonal” antibody or antigen binding fragment thereof encompasses both intact and full-length monoclonal antibodies as well as antibody fragments (such as Fab, Fab′, F(ab′)₂, Fv), single chain (scFv) mutants, fusion proteins comprising an antibody portion, and any other modified immunoglobulin molecule comprising an antigen recognition site. Furthermore, “monoclonal” antibody or antigen binding fragment thereof refers to such antibodies and antigen binding fragments thereof made in any number of manners including but not limited to by hybridoma, phage selection, recombinant expression, and transgenic animals.

The term “humanized” antibody or antigen binding fragment thereof refers to forms of non-human (e.g., murine) antibodies or antigen binding fragments that are specific immunoglobulin chains, chimeric immunoglobulins, or fragments thereof that contain minimal non-human (e.g., murine) sequences. Typically, humanized antibodies or antigen binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, certain Fv framework region (FR) residues of a human immunoglobulin are replaced with the corresponding residues in an antibody or fragment from a non-human species that has the desired specificity, affinity, and capability. The humanized antibody or antigen binding fragment thereof can be further modified by the substitution of additional residues either in the Fv framework region and/or within the non-human CDR residues to refine and optimize antibody or antigen binding fragment thereof specificity, affinity, and/or capability. In general, the humanized antibody or antigen binding fragment thereof will comprise variable domains containing all or substantially all of the CDR regions that correspond to the non-human immunoglobulin whereas all or substantially all of the FR regions are those of a human immunoglobulin consensus sequence. The humanized antibody or antigen binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-73 (1994), and Roguska et al., Protein Eng. 9(10):895-904 (1996), which are incorporated by reference herein in their entireties.

The term “human” antibody or antigen binding fragment thereof means an antibody or antigen binding fragment thereof having an amino acid sequence derived from a human immunoglobulin gene locus, where such antibody or antigen binding fragment is made using any technique known in the art. This definition of a human antibody or antigen binding fragment thereof includes intact or full-length antibodies and fragments thereof.

As used herein, the term “extracellular matrix” (also known as ECM) refers to any non-cellular component present within tissues and organs. Extracellular matrix provides physical scaffolding for cellular constituents and plays a role in many crucial biochemical and biomechanical processes. Examples of extracellular matrix include, but are not limited to, collagen, elastin, fibronectin, fibrillin, laminin, tenascin, vitronectin, osteonectin, biglycan, agrecan, versican, and neurocan.

As used herein, the term “endothelial cell adhesion molecule” refers to any protein that facilitates cell-to-cell contact between an endothelial cell and another cell (e.g., a leukocyte). Examples of an endothelial cell adhesion molecule include, but are not limited to, an intercellular adhesion molecule (ICAM) (e.g., intercellular adhesion molecule-1 (ICAM-1), intercellular adhesion molecule-2 (ICAM-2), and intercellular adhesion molecule-3 (ICAM-3)), a vascular cell adhesion molecule (VCAM) (e.g., vascular cell adhesion molecule-1 (VCAM-1)), or a selectin (e.g., E-selectin or P-selectin).

As used herein, the term “treating” includes abrogating, inhibiting, slowing or reversing the progression of a condition, substantially ameliorating clinical or aesthetical symptoms of a condition or substantially preventing the appearance of clinical or aesthetical symptoms of a condition.

The terms “administer,” “administering,” “administration,” and the like, as used herein, refer to methods that can be used to enable delivery of an antibody or antigen binding fragment thereof to the desired site of biological action (e.g., intravenous or oral administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa. and Matucci, A. et al., Respiratory Research, 19(1):154 (2018).

The terms “subject” and “patient” are used interchangeably and include any animal. Mammals are preferred, including companion (e.g., cat, dog) and farm mammals (e.g., pig, horse, cow), as well as rodents, including mice, rabbits, and rats, guinea pigs, and other rodents. Non-human primates are more preferred, and human are highly preferred.

By “specifically binds,” it is generally meant that an antibody or fragment, variant, or derivative thereof binds to an antigen or epitope by its antigen binding domain, and that the binding entails some complementarity between the antigen binding domain and the antigen or epitope. According to this definition, an antibody or fragment, variant, or derivative thereof is said to “specifically bind” to an antigen or epitope when it binds to that antigen or epitope via its antigen binding domain more readily than it would bind to a random, unrelated epitope.

An “inflammatory cell” is any cell that is part of a subject's natural defense response to injury or disease. An inflammatory cell includes, but is not limited to, a leukocyte, lymphoid cell, myeloid cell, lymphocyte, granulocyte, B cell, T cell, NK cell, neutrophil, eosinophil, basophil, mast cell, monocyte, dendritic cell, macrophage, or the like.

The term “percent identity,” as known in the art, is a relationship between two or more polypeptide sequences or two or more polynucleotide sequences, as determined by comparing the sequences. In the art, “identity” and “sequence identity” also mean the degree of sequence relatedness between polypeptide or polynucleotide sequences, as the case may be, as determined by the match between strings of such sequences. “Identity” and “similarity” can be readily calculated by known methods and publicly available resources, including but not limited to those described in: (1) Computational Molecular Biology (Lesk, A. M., Ed.) Oxford University: NY (1988); (2) Biocomputing: Informatics and Genome Projects (Smith, D. W., Ed.) Academic: NY (1993); (3) Computer Analysis of Sequence Data, Part I (Griffin, A. M., and Griffin, H. G., Eds.) Humania: NJ (1994); (4) Sequence Analysis in Molecular Biology (von Heinje, G., Ed.) Academic (1987); and (5) Sequence Analysis Primer (Gribskov, M. and Devereux, J., Eds.) Stockton: NY (1991), which are incorporated by reference herein in their entireties.

As used in the present disclosure and claims, the singular forms “a,” “an,” and “the” include plural forms unless the context clearly dictates otherwise.

It is understood that wherever aspects of the present disclosure are described herein with the language “comprising,” otherwise analogous aspects described in terms of “consisting of” and/or “consisting essentially of” are also provided.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of ±10% of the value or range remain within the intended meaning of the recited value or range. As is understood by one skilled in the art, reference to “about” a value or range herein includes (and describes) instances that are directed to that value or range per se. For example, description referring to “about X” includes description of “X.”

Any agents, antibodies, compositions, kits or methods provided herein can be combined with one or more agents, antibodies, compositions, kits or methods provided herein.

Throughout this application, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Agent Capable of Modulating the Interaction of MOSPD2 and CD63, the Interaction of MOSPD2 and Integrin β2, the Interaction of CD63 and Integrin β2, or the Interaction of a Complex of MOSPD2, Integrin β2, and CD63.

In some aspects, the disclosure provides an agent capable of modulating the interaction of MOSPD2 and CD63. In some aspects, the agent capable of modulating the interaction of MOSPD2 and CD63 is a polypeptide, DNA or RNA. In some aspects, the disclosure provides an agent capable of modulating the interaction of MOSPD2 and integrin β2. In some aspects, the agent capable of modulating the interaction of MOSPD2 and integrin β2 is a polypeptide, DNA or RNA. In some aspects, the disclosure provides an agent capable of modulating the interaction of CD63 and integrin β2. In some aspects, the agent capable of modulating the interaction of CD63 and integrin β2 is a polypeptide, DNA or RNA. In some aspects, the disclosure provides an agent capable of modulating the interaction of a complex of MOSPD2, integrin β2, and CD63. In some aspects, the agent capable of modulating the interaction of a complex of MOSPD2, integrin β2, and CD63 is a polypeptide, DNA or RNA.

As used herein, “modulating” an interaction means reducing, blocking, inhibiting, increasing, promoting, or activating the interaction (or the like), either directly or indirectly.

In some aspects, the DNA or RNA agent capable of modulating the interaction of MOSPD2 and CD63 is an antisense DNA, decoy DNA, double-stranded DNA, single-stranded DNA, complexed DNA, encapsulated DNA, viral DNA, plasmid DNA, RNAi, miRNA, siRNA, shRNA, antisense RNA, naked RNA, encapsulated RNA, viral RNA, double-stranded RNA, a molecule capable of generating RNA interference, or a gene editing system targeting MOSPD2 and/or CD63. In some aspects, the gene editing system is a CRISPR system (e.g., a CRISPR/CAS system or CRISPR/CAS9 system).

In some aspects, the DNA or RNA agent capable of modulating the interaction of MOSPD2 and integrin β2 is an antisense DNA, decoy DNA, double-stranded DNA, single-stranded DNA, complexed DNA, encapsulated DNA, viral DNA, plasmid DNA, RNAi, miRNA, siRNA, shRNA, antisense RNA, naked RNA, encapsulated RNA, viral RNA, double-stranded RNA, a molecule capable of generating RNA interference, or a gene editing system targeting MOSPD2 and/or CD63. In some aspects, the gene editing system is a CRISPR system (e.g., a CRISPR/CAS system or CRISPR/CAS9 system).

In some aspects, the DNA or RNA agent capable of modulating the interaction of CD63 and integrin β2 is an antisense DNA, decoy DNA, double-stranded DNA, single-stranded DNA, complexed DNA, encapsulated DNA, viral DNA, plasmid DNA, RNAi, miRNA, siRNA, shRNA, antisense RNA, naked RNA, encapsulated RNA, viral RNA, double-stranded RNA, a molecule capable of generating RNA interference, or a gene editing system targeting CD63 and/or integrin β2. In some aspects, the gene editing system is a CRISPR system (e.g., a CRISPR/CAS system or CRISPR/CAS9 system).

In some aspects, the DNA or RNA agent capable of modulating the interaction of a complex of MOSPD2, integrin β2, and CD63 is an antisense DNA, decoy DNA, double-stranded DNA, single-stranded DNA, complexed DNA, encapsulated DNA, viral DNA, plasmid DNA, RNAi, miRNA, siRNA, shRNA, antisense RNA, naked RNA, encapsulated RNA, viral RNA, double-stranded RNA, a molecule capable of generating RNA interference, or a gene editing system targeting MOSPD2, integrin β2, and/or CD63. In some aspects, the gene editing system is a CRISPR system (e.g., a CRISPR/CAS system or CRISPR/CAS9 system).

DNA and RNA agents capable of modulating the interaction of MOSPD2 and CD63, DNA and RNA agents capable of modulating the interaction of MOSPD2 and integrin β2, DNA and RNA agents capable of modulating the interaction of CD63 and integrin β2, and DNA and RNA agents capable of modulating the interaction of a complex of MOSPD2, integrin β2, and CD63, and methods of making the same have been described. General methods for making inhibitory DNA and RNA agents are known and described, for example, in Agrawal et al., RNA Interference: Biology, Mechanism, and Applications, Microbiol Mol Biol Rev, 2003 December; 67(4):657-685; and Dong et al., Comprehensive Biotechnology (Second Edition), 2011, Chapter 3.08.5.3 “Downregulating the Expression of Target Protein by Antisense RNA,” which are incorporated by reference herein in their entireties.

Examples of DNA and RNA agents capable of modulating interactions involving integrin β2 are known and described, for example, in Olivera et al., Braz J Med Biol Res, January 2010, Volume 43(1) 25-35, which is incorporated by reference herein in its entirety.

Examples of DNA and RNA agents capable of modulating interactions involving CD63 are also known and described, for example, in Jang et al., Exp Mol Med, 2003 Aug. 31; 35(4):317-23; and Hallden et al., J Biol Chem, 1999 Sep. 24; 274(39):27914-24, which are incorporated by reference herein in their entireties.

Examples of DNA and RNA agents capable of modulating interactions involving MOSPD2 are also known and described, for example, in Int'l Appl. Nos. PCT/IB2016/054582, PCT/IB2016/054584 and PCT/IB2019/052049 and U.S. Prov. Appl. No. 63/076,697, which are incorporated by reference herein in their entireties.

In some aspects, a polypeptide agent capable of modulating the interaction of MOSPD2 and CD63 is an anti-MOSPD2 antibody, anti-CD63 antibody, or an antigen binding fragment thereof. In some aspects, a polypeptide agent capable of modulating the interaction of MOSPD2 and integrin β2 is an anti-MOSPD2 antibody, anti-integrin β2 antibody, or an antigen binding fragment thereof. In some aspects, a polypeptide agent capable of modulating the interaction of CD63 and integrin β2 is an anti-CD63 antibody, anti-integrin β2 antibody, or an antigen binding fragment thereof. In some aspects, a polypeptide agent capable of modulating the interaction of a complex of MOSPD2, integrin β2, and CD63 is an anti-MOSPD2 antibody, anti-integrin β2 antibody, anti-CD63 antibody, or an antigen binding fragment thereof.

Polypeptide agents capable of modulating the interaction of MOSPD2 and CD63, polypeptide agents capable of modulating the interaction of MOSPD2 and integrin β2, polypeptide agents capable of modulating the interaction of CD63 and integrin β2, and polypeptide agents capable of modulating the interaction of a complex of MOSPD2, integrin β2, and CD63, and methods of making the same have been described. General methods for making inhibitory polypeptide agents and antibodies are known and described, for example in “Monoclonal Antibody Production,” National Research Council (US) Committee on Methods of Producing Monoclonal Antibodies, National Academies Press, 1999; and Carvalho et al., “Production Processes for Monoclonal Antibodies” in Fermentation Processes, IntechOpen, 2017, which are incorporated by reference herein in their entireties.

Examples of polypeptide agents capable of modulating interactions involving integrin β2 and anti-integrin β2 antibodies are known and described, for example, in Baran et al., Circulation, 2001 Dec. 4; 104(23):2778-83; and Hildreth et al., Mol Immunol, 1989 December; 26(12):1155-67, which are incorporated by reference herein in their entireties.

Examples of polypeptide agents capable of modulating interactions involving CD63 and anti-CD63 antibodies are also known and described, for example, in Wansook et al., Mol Immunol, 2019 October; 114:591-599; and Kraft et al., J Exp Med, 2005 Feb. 7; 201(3):385-96, which are incorporated by reference herein in their entireties.

Examples of polypeptide agents capable of modulating interactions involving MOSPD2 and anti-MOSPD2 antibodies are also known and described, for example, in Int'l Appl. Nos. PCT/IB2016/054582, PCT/IB2016/054584 and PCT/IB2019/052049 and U.S. Prov. Appl. No. 63/076,697, which are incorporated by reference herein in their entireties.

In some aspects, the anti-MOSPD2 antibody, anti-integrin β2 antibody, and/or anti-CD63 antibody is a polyclonal, monoclonal, murine, human, humanized, chimeric or single chain antibody.

In some aspects, the antigen binding fragment of an anti-MOSPD2 antibody, anti-integrin β2 antibody, and/or anti-CD63 antibody is a Fab, Fab′, F(ab′)₂, Fv, scFv, sdFv fragment, VH domain, or VL domain.

In some aspects, the anti-MOSPD2 antibody, anti-integrin β2 antibody, anti-CD63 antibody, or antigen binding fragment thereof specifically binds to MOSPD2, integrin β2, or CD63 with an antibody-antigen equilibrium dissociation constant (K_{D}) of from about 10⁻⁶ M to about 10⁻¹² M, or any range of values thereof (e.g., from about 10⁻⁷ M to about 10⁻¹², from 108 M to about 10⁻¹² M, from about 10⁻⁹ M to about 10⁻¹² M, from about 10⁻¹⁰ M to about 10⁻¹² M, from about 10⁻¹¹ M to about 10⁻¹² M, from about 10⁻⁶ M to about 10⁻¹¹ M, from about 10⁻⁷ M to about 10⁻¹¹ M, from about 10⁻⁸ M to about 10⁻¹¹ M, from about 10⁻⁹ M to about 10⁻¹¹ M, from about 10⁻¹⁰ M to about 10⁻¹¹ M, from about 10⁻⁶ M to about 10⁻¹⁰ M, from about 10⁻⁷ M to about 10⁻¹⁰ M, from about 10⁻⁸ M to about 10⁻¹⁰ M, from about 10⁻⁹ M to about 10⁻¹⁰ M, from about 10⁻⁶ M to about 10⁻⁹ M, from about 10⁻⁷ M to about 10⁻⁹ M, from about 10⁻⁸ M to about 10⁻⁹ M, from about 10⁻⁶ M to about 10⁻⁸ M, or from about 10⁻⁷ M to about 10⁻⁸). In some aspects, the anti-MOSPD2 antibody, anti-integrin β2 antibody, anti-CD63 antibody, or antigen binding fragment thereof specifically binds to MOSPD2, integrin β2, or CD63 with a K_{D} of about 10⁻⁶ M, about 10⁻⁷ M, about 10⁻⁸ M, about 10⁻⁹ M, about 10⁻¹⁰ M, about 10⁻¹¹ M, or about 10⁻¹² M.

In some aspects, the anti-MOSPD2 antibody, anti-integrin β2 antibody, anti-CD63 antibody, or antigen binding fragment thereof specifically binds to MOSPD2, integrin β2, or CD63 with a Kₒₙ of from about 10³ 1/Ms to about 10⁶ 1/Ms, or any range of values thereof (e.g., from about 10³ 1/Ms to about 10⁵ 1/Ms, from about 10⁴ 1/Ms to about 10⁵ 1/Ms, from about 10⁴ 1/Ms to about 10⁶ 1/Ms, from about 10⁵ 1/Ms to about 10⁶ 1/Ms, or from about 10³/Ms to about 10⁴ 1/Ms). In some aspects, the anti-MOSPD2 antibody, anti-integrin β2 antibody, anti-CD63 antibody, or antigen binding fragment thereof specifically binds to MOSPD2, integrin β2, or CD63 with a Kₒₙ of about 10³ 1/Ms, about 10⁴ 1/Ms, about 10⁵ 1/Ms, or about 10⁶ 1/Ms.

In some aspects, the anti-MOSPD2 antibody, anti-integrin β2 antibody, anti-CD63 antibody, or antigen binding fragment thereof specifically binds to MOSPD2, integrin β2, or CD63 with a K_{off} of from about 10⁻³ 1/s to about 10⁻⁶ 1/s, or any range of values thereof (e.g., from about 10⁻³ 1/s to about 10⁻⁵ 1/s, from about 10⁻⁴ 1/s to about 10⁻⁵ 1/s, from about 10⁻⁴ 1/s to about 10⁻⁶ 1/s, from about 10⁻⁵ 1/s to about 10⁻⁶ 1/s, or from about 10^{-3 1}/s to about 10⁻⁴ 1/s). In some aspects, the anti-MOSPD2 antibody, anti-integrin β2 antibody, anti-CD63 antibody, or antigen binding fragment thereof specifically binds to MOSPD2, integrin β2, or CD63 with a K_{off} of about 10⁻³ 1/s, about 10⁻⁴ 1/s, about 10^{-5 1}/s, or about 10⁻⁶ 1/s.

In some aspects, the anti-MOSPD2 antibody or antigen binding fragment thereof specifically binds to a MOSPD2 polypeptide having a sequence at least 75%, at least 80%, at least 85%, at least 90%, at least 95%, at least 96%, at least 97%, at least 98%, at least 99%, or 100% identical to any one of SEQ ID NOs:1⁻⁴. In other aspects, the anti-MOSPD2 antibody or antigen binding fragment thereof specifically binds to a MOSPD2 polypeptide encoded by a sequence at least 75%, at least 80%, at least 85%, at least 90%, at least 95%, at least 96%, at least 97%, at least 98%, at least 99%, or 100% identical to any one of SEQ ID NOs:5⁻⁸.

In some aspects, the anti-integrin β2 antibody or antigen binding fragment thereof specifically binds to integrin β2 having a sequence at least 75%, at least 80%, at least 85%, at least 90%, at least 95%, at least 96%, at least 97%, at least 98%, at least 99%, or 100% identical to any one of SEQ ID NOs:9⁻¹¹.

In some aspects, the anti-CD63 antibody or antigen binding fragment thereof specifically binds to CD63 having a sequence at least 75%, at least 80%, at least 85%, at least 90%, at least 95%, at least 96%, at least 97%, at least 98%, at least 99%, or 100% identical to SEQ ID NO:12.

In some aspects, the agent capable of modulating the interaction of MOSPD2 and CD63 is not an oxidized phospholipid. In some aspects, the agent capable of modulating the interaction of MOSPD2 and CD63 is not 1-hexadecyl-2-(4′-carboxy)butyl-glycero-3-phosphocholine (VB-201).

In some aspects, the agent capable of modulating the interaction of MOSPD2 and integrin β2 is not an oxidized phospholipid. In some aspects, the agent capable of modulating the interaction of MOSPD2 and integrin β2 is not 1-hexadecyl-2-(4′-carboxy)butyl-glycero-3-phosphocholine (VB-201).

In some aspects, the agent capable of modulating the interaction of CD63 and integrin β2 is not an oxidized phospholipid. In some aspects, the agent capable of modulating the interaction of CD63 and integrin β2 is not 1-hexadecyl-2-(4′-carboxy)butyl-glycero-3-phosphocholine (VB-201).

In some aspects, the agent capable of modulating the interaction of a complex of MOSPD2, integrin β2, and CD63 is not an oxidized phospholipid. In some aspects, the agent capable of modulating the interaction of a complex of MOSPD2, integrin β2, and CD63 is not VB-201.

In some aspects, the disclosure provides a composition comprising an agent capable of modulating the interaction of MOSPD2 and CD63, as described herein. In some aspects, the composition comprises an agent capable of modulating the interaction of MOSPD2 and CD63, as described herein, and a carrier.

In some aspects, the disclosure provides a composition comprising an agent capable of modulating the interaction of MOSPD2 and integrin β2, as described herein. In some aspects, the composition comprises an agent capable of modulating the interaction of MOSPD2 and integrin β2, as described herein, and a carrier.

In some aspects, the disclosure provides a composition comprising an agent capable of modulating the interaction of CD63 and integrin β2, as described herein. In some aspects, the composition comprises an agent capable of modulating the interaction of CD63 and integrin β2, as described herein, and a carrier.

In some aspects, the disclosure provides a composition comprising an agent capable of modulating the interaction of a complex of MOSPD2, integrin β2, and CD63, as described herein. In some aspects, the composition comprises an agent capable of modulating the interaction of a complex of MOSPD2, integrin β2, and CD63, as described herein, and a carrier.

In some aspects, the carrier is a diluent, adjuvant, excipient, or vehicle with which the agent is administered. Examples of a carrier include, but are not limited to, liquids, such as water and oils. Water, aqueous solution saline, and aqueous dextrose and glycerol solutions can also be employed as carriers, particularly for injectable solutions.

In some aspects, the composition is formulated for oral administration. Examples of such a composition include, but are not limited to, tablets and capsules.

In some aspects, the composition is formulated for administration as an injection or infusion. Routes of administration by injection or infusion include intravenous, intraperitoneal, intramuscular, intrathecal and subcutaneous.

In some aspects, the disclosure provides a kit comprising (i) an agent capable of modulating the interaction of MOSPD2 and CD63, or other antibody, antigen binding fragment, or composition as described herein; and (ii) an instruction for use. In some aspects, the disclosure provides a kit comprising (i) an agent capable of modulating the interaction of MOSPD2 and integrin β2, or other antibody, antigen binding fragment, or composition as described herein; and (ii) an instruction for use. In some aspects, the disclosure provides a kit comprising (i) an agent capable of modulating the interaction of CD63 and integrin β2, or other antibody, antigen binding fragment, or composition as described herein; and (ii) an instruction for use. In some aspects, the disclosure provides a kit comprising (i) an agent capable of modulating the interaction of a complex of MOSPD2, integrin β2 and CD63, or other antibody, antigen binding fragment, or composition as described herein; and (ii) an instruction for use. In some aspects, the instruction for use is for any method described herein (e.g., for treating or preventing an inflammatory disease or disorder or treating, preventing or reducing the incidence of cancer or cancer metastasis).

In some aspects, the disclosure provides a composition comprising an agent capable of modulating the interaction of MOSPD2 and CD63, as described herein, and one or more additional active agents. In some aspects, the disclosure provides a composition comprising an agent capable of modulating the interaction of MOSPD2 and integrin β2, as described herein, and one or more additional active agents. In some aspects, the disclosure provides a composition comprising an agent capable of modulating the interaction of CD63 and integrin β2, as described herein, and one or more additional active agents. In some aspects, the disclosure provides a composition comprising an agent capable of modulating the interaction of a complex of MOSPD2, integrin β2, and CD63, as described herein, and one or more additional active agents.

### Methods of Use

In some aspects, the disclosure provides methods of use for an agent capable of modulating the interaction of MOSPD2 and CD63, as described herein. In some aspects, the disclosure provides methods of use for an agent capable of modulating the interaction of MOSPD2 and integrin β2, as described herein. In some aspects, the disclosure provides methods of use for an agent capable of modulating the interaction of CD63 and integrin β2, as described herein. In some aspects, the disclosure provides methods of use for an agent capable of modulating the interaction of a complex of MOSPD2, integrin β2, and CD63, as described herein.

In some aspects, the disclosure provides a method of inhibiting migration of an inflammatory cell, comprising contacting the inflammatory cell with an agent capable of modulating the interaction of MOSPD2 and CD63. In some aspects, the disclosure provides a method of inhibiting migration of an inflammatory cell, comprising contacting the inflammatory cell with an agent capable of modulating the interaction of MOSPD2 and integrin β2. In some aspects, the disclosure provides a method of inhibiting migration of an inflammatory cell, comprising contacting the inflammatory cell with an agent capable of modulating the interaction of CD63 and integrin β2. In some aspects, the disclosure provides a method of inhibiting migration of an inflammatory cell, comprising contacting the inflammatory cell with an agent capable of modulating the interaction of a complex of MOSPD2, integrin β2, and CD63.

In some aspects, the disclosure provides a method of promoting adhesion of an inflammatory cell to an endothelial cell adhesion molecule (and adhesion of an endothelial cell adhesion molecule to an inflammatory cell), comprising contacting the inflammatory cell with an isolated agent capable of modulating the interaction of MOSPD2 and CD63. In some aspects, the disclosure provides a method of promoting adhesion of an inflammatory cell to an endothelial cell adhesion molecule (and adhesion of an endothelial cell adhesion molecule to an inflammatory cell), comprising contacting the inflammatory cell with an isolated agent capable of modulating the interaction of MOSPD2 and integrin β2. In some aspects, the disclosure provides a method of promoting adhesion of an inflammatory cell to an endothelial cell adhesion molecule (and adhesion of an endothelial cell adhesion molecule to an inflammatory cell), comprising contacting the inflammatory cell with an isolated agent capable of modulating the interaction of CD63 and integrin β2. In some aspects, the disclosure provides a method of promoting adhesion of an inflammatory cell to an endothelial cell adhesion molecule (and adhesion of an endothelial cell adhesion molecule to an inflammatory cell), comprising contacting the inflammatory cell with an isolated agent capable of modulating the interaction of a complex of MOSPD2, integrin β2, and CD63.

In some aspects, the disclosure provides a method of modulating adhesion of an inflammatory cell to an endothelial cell adhesion molecule (and adhesion of an endothelial cell adhesion molecule to an inflammatory cell), comprising contacting the inflammatory cell with an isolated agent capable of modulating the interaction of MOSPD2 and CD63. In some aspects, the disclosure provides a method of modulating adhesion of an inflammatory cell to an endothelial cell adhesion molecule (and adhesion of an endothelial cell adhesion molecule to an inflammatory cell), comprising contacting the inflammatory cell with an isolated agent capable of modulating the interaction of MOSPD2 and integrin β2. In some aspects, the disclosure provides a method of modulating adhesion of an inflammatory cell to an endothelial cell adhesion molecule (and adhesion of an endothelial cell adhesion molecule to an inflammatory cell), comprising contacting the inflammatory cell with an isolated agent capable of modulating the interaction of CD63 and integrin β2. In some aspects, the disclosure provides a method of modulating adhesion of an inflammatory cell to an endothelial cell adhesion molecule (and adhesion of an endothelial cell adhesion molecule to an inflammatory cell), comprising contacting the inflammatory cell with an isolated agent capable of modulating the interaction of a complex of MOSPD2, integrin β2, and CD63. In some aspects, modulating adhesion is promoting adhesion. In other aspects, modulating adhesion is inhibiting adhesion.

In some aspects, the disclosure provides a method of promoting adhesion of an inflammatory cell to an extracellular matrix component (and adhesion of an extracellular matrix component to an inflammatory cell), comprising contacting the inflammatory cell with an agent capable of modulating the interaction of MOSPD2 and CD63. In some aspects, the disclosure provides a method of promoting adhesion of an inflammatory cell to an extracellular matrix component (or adhesion of an extracellular matrix component to an inflammatory cell), comprising contacting the inflammatory cell with an agent capable of modulating the interaction of MOSPD2 and integrin β2. In some aspects, the disclosure provides a method of promoting adhesion of an inflammatory cell to an extracellular matrix component (or adhesion of an extracellular matrix component to an inflammatory cell), comprising contacting the inflammatory cell with an agent capable of modulating the interaction of CD63 and integrin β2. In some aspects, the disclosure provides a method of promoting adhesion of an inflammatory cell to an extracellular matrix component (or adhesion of an extracellular matrix component to an inflammatory cell), comprising contacting the inflammatory cell with an agent capable of modulating the interaction of a complex of MOSPD2, integrin β2, and CD63.

In some aspects, the disclosure provides a method of modulating adhesion of an inflammatory cell to an extracellular matrix component (and adhesion of an extracellular matrix component to an inflammatory cell), comprising contacting the inflammatory cell with an agent capable of modulating the interaction of MOSPD2 and CD63. In some aspects, the disclosure provides a method of modulating adhesion of an inflammatory cell to an extracellular matrix component (or adhesion of an extracellular matrix component to an inflammatory cell), comprising contacting the inflammatory cell with an agent capable of modulating the interaction of MOSPD2 and integrin β2. In some aspects, the disclosure provides a method of modulating adhesion of an inflammatory cell to an extracellular matrix component (or adhesion of an extracellular matrix component to an inflammatory cell), comprising contacting the inflammatory cell with an agent capable of modulating the interaction of CD63 and integrin β2. In some aspects, the disclosure provides a method of modulating adhesion of an inflammatory cell to an extracellular matrix component (or adhesion of an extracellular matrix component to an inflammatory cell), comprising contacting the inflammatory cell with an agent capable of modulating the interaction of a complex of MOSPD2, integrin β2, and CD63. In some aspects, modulating adhesion is promoting adhesion. In other aspects, modulating adhesion is inhibiting adhesion.

In some aspects, the disclosure provides a method of inducing expression of a protein associated with integrin activation in an inflammatory cell, comprising contacting the inflammatory cell with an isolated agent capable of modulating the interaction of MOSPD2 and CD63. In some aspects, the disclosure provides a method of inducing expression of a protein associated with integrin activation in an inflammatory cell, comprising contacting the inflammatory cell with an isolated agent capable of modulating the interaction of MOSPD2 and integrin β2. In some aspects, the disclosure provides a method of inducing expression of a protein associated with integrin activation in an inflammatory cell, comprising contacting the inflammatory cell with an isolated agent capable of modulating the interaction of CD63 and integrin β2. In some aspects, the disclosure provides a method of inducing expression of a protein associated with integrin activation in an inflammatory cell, comprising contacting the inflammatory cell with an isolated agent capable of modulating the interaction of a complex of MOSPD2, integrin β2, and CD63.

In some aspects, the inflammatory cell of the methods provided herein is a leukocyte, lymphoid cell, myeloid cell, lymphocyte, granulocyte, B cell, T cell, NK cell, neutrophil, eosinophil, basophil, mast cell, monocyte, dendritic cell, or macrophage. In some aspects, the macrophage is a tumor-associated macrophage (TAM).

In some aspects, the extracellular matrix component of the methods provided herein is collagen, elastin, fibronectin, fibrillin, laminin, tenascin, vitronectin, osteonectin, biglycan, agrecan, versican, or neurocan.

In some aspects, the endothelial cell adhesion molecule of the methods provided herein is an ICAM, VCAM, or selectin. In some aspects, the endothelial cell adhesion molecule of the methods provided herein is ICAM-1, ICAM-2, ICAM-3, VCAM-1, E-selectin, or P-selectin.

In some aspects, the protein associated with integrin activation of the methods provided herein is phosphorylated FAK (p-FAK), phosphorylated AKT (p-AKT), phosphorylated ERK1/2 (p-ERK1/2), or phosphorylated P38 (p-P38).

In some aspects, the disclosure provides a method of treating or preventing an inflammatory disease or disorder, comprising administering a therapeutically effective amount of agent capable of modulating the interaction of MOSPD2 and CD63 to a subject in need thereof. In some aspects, the disclosure provides a method of treating or preventing an inflammatory disease or disorder, comprising administering a therapeutically effective amount of agent capable of modulating the interaction of MOSPD2 and integrin β2 to a subject in need thereof. In some aspects, the disclosure provides a method of treating or preventing an inflammatory disease or disorder, comprising administering a therapeutically effective amount of agent capable of modulating the interaction of CD63 and integrin β2 to a subject in need thereof. In some aspects, the disclosure provides a method of treating or preventing an inflammatory disease or disorder, comprising administering a therapeutically effective amount of agent capable of modulating the interaction of a complex of MOSPD2, integrin β2, and CD63 to a subject in need thereof.

In some aspects, the inflammatory disease or disorder of the methods provided herein is an idiopathic inflammatory disease or disorder, a chronic inflammatory disease or disorder, an acute inflammatory disease or disorder, an autoimmune disease or disorder, an infectious disease or disorder, an inflammatory malignant disease or disorder, an inflammatory transplantation-related disease or disorder, an inflammatory degenerative disease or disorder, a disease or disorder associated with a hypersensitivity, an inflammatory cardiovascular disease or disorder, an inflammatory cerebrovascular disease or disorder, a peripheral vascular disease or disorder, an inflammatory glandular disease or disorder, an inflammatory gastrointestinal disease or disorder, an inflammatory cutaneous disease or disorder, an inflammatory hepatic disease or disorder, an inflammatory neurological disease or disorder, an inflammatory musculo-skeletal disease or disorder, an inflammatory renal disease or disorder, an inflammatory reproductive disease or disorder, an inflammatory systemic disease or disorder, an inflammatory connective tissue disease or disorder, necrosis, an inflammatory implant-related disease or disorder, an inflammatory aging process, an immunodeficiency disease or disorder, or an inflammatory pulmonary disease or disorder.

In some aspects, the hypersensitivity is Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity, delayed type hypersensitivity, helper T lymphocyte mediated hypersensitivity, cytotoxic T lymphocyte mediated hypersensitivity, TH1 lymphocyte mediated hypersensitivity, or TH2 lymphocyte mediated hypersensitivity.

In some aspects, the inflammatory cardiovascular disease or disorder is an occlusive disease or disorder, atherosclerosis, a cardiac valvular disease, stenosis, restenosis, in-stent-stenosis, myocardial infarction, coronary arterial disease, acute coronary syndromes, congestive heart failure, angina pectoris, myocardial ischemia, thrombosis, Wegener's granulomatosis, Takayasu's arteritis, Kawasaki syndrome, anti-factor VIII autoimmune disease or disorder, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity, Chagas' disease or disorder, or anti-helper T lymphocyte autoimmunity.

In some aspects, the inflammatory cerebrovascular disease or disorder is stroke, cerebrovascular inflammation, cerebral hemorrhage, or vertebral arterial insufficiency.

In some aspects, the peripheral vascular disease or disorder is gangrene, diabetic vasculopathy, ischemic bowel disease, thrombosis, diabetic retinopathy, or diabetic nephropathy.

In some aspects, the autoimmune disease or disorder is chronic rheumatoid arthritis, juvenile rheumatoid arthritis, systemic lupus erythematosus, scleroderma, mixed connective tissue disease, polyarteritis nodosa, polymyositis/dermatomyositis, Sjogren's syndrome, Bechet's disease, autoimmune diabetes, Hashimoto's disease, psoriasis, primary myxedema, pernicious anemia, myasthenia gravis, chronic active hepatitis, autoimmune hemolytic anemia, idiopathic thrombocytopenic purpura, uveitis, vasculitides, or heparin induced thrombocytopenia.

In some aspects, the inflammatory glandular disease or disorder is a pancreatic disease or disorder, Type I diabetes, thyroid disease or disorder, Graves' disease or disorder, thyroiditis, spontaneous autoimmune thyroiditis, Hashimoto's thyroiditis, idiopathic myxedema, ovarian autoimmunity, autoimmune anti-sperm infertility, autoimmune prostatitis, or Type I autoimmune polyglandular syndrome.

In some aspects, the inflammatory gastrointestinal disease or disorder is colitis, ileitis, Crohn's disease, chronic inflammatory intestinal disease, inflammatory bowel syndrome, inflammatory bowel disease, celiac disease, ulcerative colitis, an ulcer, a skin ulcer, a bed sore, a gastric ulcer, a peptic ulcer, a buccal ulcer, a nasopharyngeal ulcer, an esophageal ulcer, a duodenal ulcer, or a gastrointestinal ulcer.

In some aspects, the inflammatory cutaneous disease or disorder is acne, autoimmune bullous skin disease or disorder, pemphigus vulgaris, bullous pemphigoid, pemphigus foliaceus, contact dermatitis, or drug eruption.

In some aspects, the inflammatory hepatic disease or disorder is autoimmune hepatitis, hepatic cirrhosis, or biliary cirrhosis.

In some aspects, the inflammatory neurological disease or disorder is multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease or disorder, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, Huntington's disease, AIDS associated dementia, amyotrophic lateral sclerosis, multiple sclerosis, stroke, an inflammatory retinal disease or disorder, an inflammatory ocular disease or disorder, optic neuritis, spongiform encephalopathy, migraine, headache, cluster headache, or stiff-man syndrome.

In some aspects, the multiple sclerosis is relapsing-remitting multiple sclerosis (RRMS), primary progressive multiple sclerosis (PPMS), or secondary progressive multiple sclerosis (SPMS).

In some aspects, the inflammatory connective tissue disease or disorder is Duchenne muscular dystrophy (DMD), autoimmune myositis, primary Sjogren's syndrome, smooth muscle autoimmune disease or disorder, myositis, tendinitis, a ligament inflammation, chondritis, a joint inflammation, a synovial inflammation, carpal tunnel syndrome, arthritis, ankylosing spondylitis, a skeletal inflammation, an autoimmune ear disease or disorder, or an autoimmune disease or disorder of the inner ear.

In some aspects, the arthritis is rheumatoid arthritis, osteoarthritis, psoriatic arthritis, juvenile arthritis, chronic rheumatoid arthritis, or juvenile rheumatoid arthritis.

In some aspects, the inflammatory renal disease or disorder is autoimmune interstitial nephritis.

In some aspects, the inflammatory reproductive disease or disorder is repeated fetal loss, ovarian cyst, or a menstruation associated disease or disorder.

In some aspects, the inflammatory systemic disease or disorder is systemic lupus erythematosus, systemic sclerosis, septic shock, toxic shock syndrome, or cachexia.

In some aspects, the infectious disease or disorder is a chronic infectious disease or disorder, a subacute infectious disease or disorder, an acute infectious disease or disorder, a viral disease or disorder, a bacterial disease or disorder, a protozoan disease or disorder, a parasitic disease or disorder, a fungal disease or disorder, a mycoplasma disease or disorder, gangrene, sepsis, a prion disease or disorder, influenza, tuberculosis, malaria, acquired immunodeficiency syndrome, or severe acute respiratory syndrome.

In some aspects, the inflammatory transplantation-related disease or disorder is graft rejection, chronic graft rejection, subacute graft rejection, acute graft rejection hyperacute graft rejection, or graft versus host disease or disorder.

In some aspects, the implant is a prosthetic implant, a breast implant, a silicone implant, a dental implant, a penile implant, a cardiac implant, an artificial joint, a bone fracture repair device, a bone replacement implant, a drug delivery implant, a catheter, a pacemaker, an artificial heart, an artificial heart valve, a drug release implant, an electrode, or a respirator tube.

In some aspects, the inflammatory pulmonary disease or disorder is asthma, allergic asthma, emphysema, chronic obstructive pulmonary disease or disorder, sarcoidosis, or bronchitis.

In some aspects, the inflammatory disease or disorder is fibrosis.

In some aspects, the inflammatory disease or disorder is vascular inflammation in a subject suffering from a chronic autoimmune or chronic inflammatory disease.

In some aspects, the chronic autoimmune or inflammatory disease is psoriasis.

In some aspects, the vascular inflammation is associated with a cardiovascular disease, a peripheral vascular disease, a coronary artery disease, a cerebral vascular disease, a renal artery stenosis, an ischemic disease, or an aortic aneurism.

In some aspects, the vascular inflammation is associated with an ischemic heart disease, atherosclerosis, acute coronary syndrome, unstable angina, stable angina, or stroke.

In some aspects, the vascular inflammation is inflammation of a carotid artery or inflammation of an aorta.

In some aspects, the inflammatory disease or disorder is inflammation associated with an implant. In some aspects, the inflammation associated with an implant is a local inflammation or a systemic inflammatory reaction. In some aspects, the implant is a silicone implant, a saline implant, a metal implant, a plastic implant, a polymeric implant, a cosmetic implant, a prosthetic implant, a subdermal implant, a transdermal implant, a bone replacement implant, a bone fracture repair device, a drug delivery implant, a drug release implant, an artificial joint, an artificial heart, an artificial heart valve, a testicular prosthesis, a breast implant, a dental implant, an ocular implant, a cochlear implant, a penile implant, a cardiac implant, a catheter, an implantable urinary continence device, a pacemaker, an electrode, a Hernia support device, or a respirator tube.

In some aspects, the inflammatory disease or disorder is hepatitis, steatohepatitis, nonalcoholic steatohepatitis (NASH), glomerulonephritis, focal segmental glomerulosclerosis (FSGS), or osteoporosis.

In some aspects, the disclosure provides a method of treating or preventing cancer, comprising administering a therapeutically effective amount of an agent capable of modulating the interaction of MOSPD2 and CD63 to a subject in need thereof. In some aspects, the disclosure provides a method of treating or preventing cancer, comprising administering a therapeutically effective amount of an agent capable of modulating the interaction of MOSPD2 and integrin β2 to a subject in need thereof. In some aspects, the disclosure provides a method of treating or preventing cancer, comprising administering a therapeutically effective amount of an agent capable of modulating the interaction of CD63 and integrin β2 to a subject in need thereof. In some aspects, the disclosure provides a method of treating or preventing cancer, comprising administering a therapeutically effective amount of an agent capable of modulating the interaction of a complex of MOSPD2, integrin β2, and CD63 to a subject in need thereof.

In some aspects, the disclosure provides a method of treating, preventing or reducing the incidence of cancer metastasis, comprising administration of a therapeutically effective amount of an agent capable of modulating the interaction of MOSPD2 and CD63 to a subject in need thereof. In some aspects, the disclosure provides a method of treating, preventing or reducing the incidence of cancer metastasis, comprising administration of a therapeutically effective amount of an agent capable of modulating the interaction of MOSPD2 and integrin β2 to a subject in need thereof. In some aspects, the disclosure provides a method of treating, preventing or reducing the incidence of cancer metastasis, comprising administration of a therapeutically effective amount of an agent capable of modulating the interaction of CD63 and integrin β2 to a subject in need thereof. In some aspects, the disclosure provides a method of treating, preventing or reducing the incidence of cancer metastasis, comprising administration of a therapeutically effective amount of an agent capable of modulating the interaction of a complex of MOSPD2, integrin β2, and CD63 to a subject in need thereof.

In some aspects, the cancer of the methods described herein is bladder cancer, breast cancer, colon cancer, rectal cancer, kidney cancer, liver cancer, lung cancer, esophageal cancer, gall-bladder cancer, ovarian cancer, pancreatic cancer, stomach cancer, cervical cancer, thyroid cancer, prostate cancer, skin cancer, hematopoietic cancer, cancer of mesenchymal origin, cancer of central or peripheral nervous system, endometrial cancer, head and neck cancer, glioblastoma, or malignant ascites.

In some aspects, the lung cancer is a small-cell lung cancer or a non-small-cell lung cancer.

In some aspects, the skin cancer is squamous cell carcinoma, basal cell cancer, melanoma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma.

In some aspects, the cancer is a hematopoietic cancer of lymphoid lineage. In some aspects, the hematopoietic cancer of lymphoid lineage is leukemia, acute lymphocytic leukemia, chronic lymphocytic leukemia, acute lymphoblastic leukemia, B-cell lymphoma, T-cell-lymphoma, Hodgkin's lymphoma, non-Hodgkin's lymphoma, hairy cell lymphoma, or Burkitt's lymphoma.

In some aspects, the cancer is a hematopoietic cancer of myeloid lineage. In some aspects, the hematopoietic cancer of myeloid lineage is acute myelogenous leukemia, chronic myelogenous leukemia, myelodysplastic syndrome, and promyelocytic leukemia.

In some aspects, the cancer is a cancer of mesenchymal origin. In some aspects, the cancer of mesenchymal origin is fibrosarcoma, rhabdomyosarcoma, soft tissue sarcoma, or bone sarcoma.

In some aspects, the cancer is a cancer of the central or peripheral nervous system. In some aspects, the cancer of the central or peripheral nervous system is astrocytoma, neuroblastoma, glioma, or schwannomas.

In some aspects, the cancer is anal cancer, bone cancer, gastrointestinal stomal cancer, gestational trophoblastic disease, Hodgkin's lymphoma, Kaposi sarcoma, keratoacanthoma, malignant mesothelioma, multicentric castleman disease, multiple myeloma and other plasma cell neoplasms, myeloproliferative neoplasms, neuroblastoma, non-Hodgkin's lymphoma, osteosarcoma, ovarian, fallopian tube, or primary peritoneal cancer, penile cancer, retinoblastoma, rhabdomyosarcoma, seminoma, soft tissue sarcoma, stomach (gastric) cancer, testicular cancer, teratocarcinoma, thyroid follicular cancer, vaginal cancer, vulvar cancer, Wilms tumor and other childhood kidney cancers, or xeroderma pigmentosum.

In some aspects, the cancer is bladder cancer, brain cancer, breast cancer, colon cancer, esophageal cancer, lung cancer, skin cancer, tongue cancer, kidney cancer, or hepatic cancer.

In some aspects, ERK phosphorylation in a cancer cell of the methods provided herein is inhibited, AKT phosphorylation in a cancer cell of the methods of provided herein is inhibited, and/or FAK phosphorylation in a cancer cell of the methods provided herein is inhibited.

In some aspects, the methods of treating, preventing or reducing the incidence of cancer or cancer metastasis provided herein further comprise administering a therapeutically effective amount of an anticancer agent. In some aspects, the anticancer agent is Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and I 131 Iodine Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPOX, Carboplatin, Carboplatin-Taxol, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, Chlorambucil-Prednisone, CHOP, Cisplatin, Clafen (Cyclophosphamide), Clofarabine, CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), Folfiri, Folfiri-Bevacizumab, Folfiri-Cetuximab, Folfirinox, Folflox, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, OEPA, Ofatumumab, OFF, Olaparib, Omacetaxine Mepesuccinate, Oncaspar (Pegaspargase), Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and 1131 Iodine Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), Zydelig (Idelalisib), Zykadia (Ceritinib), or Zytiga (Abiraterone Acetate).

In some aspects, the disclosure provides a method for inducing a conformational change in an integrin, comprising contacting an inflammatory cell or a subject in need thereof with an agent capable of modulating the interaction of MOSPD2 and CD63. In some aspects, the disclosure provides a method for inducing a conformational change in an integrin, comprising contacting an inflammatory cell or a subject in need thereof with an agent capable of modulating the interaction of MOSPD2 and integrin β2. In some aspects, the disclosure provides a method for inducing a conformational change in an integrin, comprising contacting an inflammatory cell or a subject in need thereof with an agent capable of modulating the interaction of CD63 and integrin β2. In some aspects, the disclosure provides a method for inducing a conformational change in an integrin, comprising contacting an inflammatory cell or a subject in need thereof with an agent capable of modulating the interaction of a complex of MOSPD2, integrin β2, and CD63.

In some aspects, the disclosure provides a method for inducing a transition in an integrin from low to high binding affinity conformation, comprising contacting an inflammatory cell or a subject in need thereof with an agent capable of modulating the interaction of MOSPD2 and CD63. In some aspects, the disclosure provides a method for inducing a transition in an integrin from low to high binding affinity conformation, comprising contacting an inflammatory cell or a subject in need thereof with an agent capable of modulating the interaction of MOSPD2 and integrin β2. In some aspects, the disclosure provides a method for inducing a transition in an integrin from low to high binding affinity conformation, comprising contacting an inflammatory cell or a subject in need thereof with an agent capable of modulating the interaction of CD63 and integrin β2. In some aspects, the disclosure provides a method for inducing a transition in an integrin from low to high binding affinity conformation, comprising contacting an inflammatory cell or a subject in need thereof with an agent capable of modulating the interaction of a complex of MOSPD2, integrin β2, and CD63.

In some aspects, the disclosure provides a method for the prediction, diagnosis, or prognosis of an inflammatory disease or disorder, cancer, or cancer metastasis in a subject comprising determining the level of a complex of MOSPD2 and CD63 in a sample of a subject. In some aspects, the disclosure provides a method for the prediction, diagnosis, or prognosis of an inflammatory disease or disorder, cancer, or cancer metastasis in a subject comprising determining the level of a complex of MOSPD2 and integrin β2 in a sample of a subject. In some aspects, the disclosure provides a method for the prediction, diagnosis, or prognosis of an inflammatory disease or disorder, cancer, or cancer metastasis in a subject comprising determining the level of a complex of CD63 and integrin β2 in a sample of a subject. In some aspects, the disclosure provides a method for the prediction, diagnosis, or prognosis of an inflammatory disease or disorder, cancer, or cancer metastasis in a subject comprising determining the level of a complex of MOSPD2, integrin β2, and CD63 in a sample of a subject.

In some aspects, the level of the complex is determined using an agent capable of modulating the interaction of MOSPD2 and CD63, as described herein (e.g., an anti-MOSPD2 antibody and/or anti-CD63 antibody). In some aspects, the level of the complex is determined using an agent capable of modulating the interaction of MOSPD2 and integrin β2, as described herein (e.g., an anti-MOSPD2 antibody and/or anti-integrin β2 antibody). In some aspects, the level of the complex is determined using an agent capable of modulating the interaction of CD63 and integrin β2, as described herein (e.g., an anti-CD63 antibody and/or anti-integrin β2 antibody). In some aspects, the level of the complex is determined using an agent capable of modulating the interaction of a complex of MOSPD2, integrin β2, and CD63, as described herein (e.g., an anti-MOSPD2 antibody, anti-integrin β2 antibody, and/or anti-CD63 antibody).

In some aspects, the method comprises (i) determining or quantifying the level of a complex of MOSPD2 and CD63 in a sample of the subject, and (ii) comparing the level obtained in step (i) with a control or reference value, wherein an increased level of a complex of MOSPD2 and CD63 with respect to the control or reference value is indicative of an inflammatory disease or disorder, cancer, cancer metastasis, or an increased risk of an inflammatory disease or disorder, cancer, cancer metastasis.

In some aspects, the method comprises (i) determining or quantifying the level of a complex of MOSPD2 and integrin β2 in a sample of the subject, and (ii) comparing the level obtained in step (i) with a control or reference value, wherein an increased level of a complex of MOSPD2 and integrin β2 with respect to the control or reference value is indicative of an inflammatory disease or disorder, cancer, cancer metastasis, or an increased risk of an inflammatory disease or disorder, cancer, cancer metastasis.

In some aspects, the method comprises (i) determining or quantifying the level of a complex of CD63 and integrin β2 in a sample of the subject, and (ii) comparing the level obtained in step (i) with a control or reference value, wherein an increased level of a complex of CD63 and integrin β2 with respect to the control or reference value is indicative of an inflammatory disease or disorder, cancer, cancer metastasis, or an increased risk of an inflammatory disease or disorder, cancer, cancer metastasis.

In some aspects, the method comprises (i) determining or quantifying the level of a complex of MOSPD2, integrin β2, and CD63 in a sample of the subject, and (ii) comparing the level obtained in step (i) with a control or reference value, wherein an increased level of a complex of MOSPD2, integrin β2, and CD63 with respect to the control or reference value is indicative of an inflammatory disease or disorder, cancer, cancer metastasis, or an increased risk of an inflammatory disease or disorder, cancer, cancer metastasis.

In some aspects, the sample is a tissue biopsy, tumor biopsy, or blood sample from a subject.

In some aspects, the control or reference value is the level of a complex of MOSPD2 and CD63 in normal tissue (e.g., normal adjacent tissue (NAT)). In some aspects, the control or reference value is the level of a complex of MOSPD2 and integrin β2 in normal tissue (e.g., normal adjacent tissue (NAT)). In some aspects, the control or reference value is the level of a complex of CD63 and integrin β2 in normal tissue (e.g., normal adjacent tissue (NAT)). In some aspects, the control or reference value is the level of a complex of MOSPD2, integrin β2, and CD63 in normal tissue (e.g., normal adjacent tissue (NAT)).

In other aspects, the control or reference value is no detectable level of a complex of MOSPD2 and CD63 or no significant level of a complex of MOSPD2 and CD63. In other aspects, the control or reference value is no detectable level of a complex of MOSPD2 and integrin β2 or no significant level of a complex of MOSPD2 and integrin β2. In other aspects, the control or reference value is no detectable level of a complex of CD63 and integrin β2 or no significant level of a complex of CD63 and integrin β2. In other aspects, the control or reference value is no detectable level of a complex of MOSPD2, integrin β2, and CD63 or no significant level of a complex of MOSPD2, integrin β2, and CD63.

In some aspects, the agent capable of modulating the interaction of MOSPD2 and CD63 is an anti-MOSPD2 antibody, anti-CD63 antibody, or antigen binding fragment thereof, and is administered in the methods provided herein at an amount of from about 1 μg/ml to about 10 μg/ml, or any range of values thereof (e.g., from about 2 μg/ml to about 10 μg/ml, from about 3 μg/ml to about 10 μg/ml, from about 4 μg/ml to about 10 μg/ml, from about 5 μg/ml to about 10 μg/ml, from about 6 μg/ml to about 10 μg/ml, from about 7 μg/ml to about 10 μg/ml, from about 8 μg/ml to about 10 μg/ml, from about 9 μg/ml to about 10 μg/ml, from about 1 μg/ml to about 9 μg/ml, from about 2 μg/ml to about 9 μg/ml, from about 3 μg/ml to about 9 μg/ml, from about 4 μg/ml to about 9 μg/ml, from about 5 μg/ml to about 9 μg/ml, from about 6 μg/ml to about 9 μg/ml, from about 7 μg/ml to about 9 μg/ml, from about 8 μg/ml to about 9 μg/ml, from about 1 μg/ml to about 8 μg/ml, from about 2 μg/ml to about 8 μg/ml, from about 3 μg/ml to about 8 μg/ml, from about 4 μg/ml to about 8 μg/ml, from about 5 μg/ml to about 8 μg/ml, from about 6 μg/ml to about 8 μg/ml, from about 7 μg/ml to about 8 μg/ml, from about 1 μg/ml to about 7 μg/ml, from about 2 μg/ml to about 7 μg/ml, from about 3 μg/ml to about 7 μg/ml, from about 4 μg/ml to about 7 μg/ml, from about 5 μg/ml to about 7 μg/ml, from about 6 μg/ml to about 7 μg/ml, from about 1 μg/ml to about 6 μg/ml, from about 2 μg/ml to about 6 μg/ml, from about 3 μg/ml to about 6 μg/ml, from about 4 μg/ml to about 6 μg/ml, from about 5 μg/ml to about 6 μg/ml, from about 1 μg/ml to about 5 μg/ml, from about 2 μg/ml to about 5 μg/ml, from about 3 μg/ml to about 5 μg/ml, from about 4 μg/ml to about 5 μg/ml, from about 1 μg/ml to about 4 μg/ml, from about 2 μg/ml to about 4 μg/ml, from about 3 μg/ml to about 4 μg/ml, from about 1 μg/ml to about 3 μg/ml, from about 2 μg/ml to about 3 μg/ml, or from about 1 μg/ml to about 2 μg/ml). In other aspects, the agent capable of modulating the interaction of MOSPD2 and CD63 is an anti-MOSPD2 antibody, anti-CD63 antibody, or antigen binding fragment thereof, and is administered in the methods provided herein at an amount of from about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, or about 10 μg/ml.

In some aspects, the agent capable of modulating the interaction of MOSPD2 and integrin β2 is an anti-MOSPD2 antibody, anti-integrin β2 antibody, or antigen binding fragment thereof, and is administered in the methods provided herein at an amount of from about 1 μg/ml to about 10 μg/ml, or any range of values thereof (e.g., from about 2 μg/ml to about 10 μg/ml, from about 3 μg/ml to about 10 μg/ml, from about 4 μg/ml to about 10 g/ml, from about 5 g/ml to about 10 g/ml, from about 6 g/ml to about 10 μg/ml, from about 7 g/ml to about 10 μg/ml, from about 8 g/ml to about 10 μg/ml, from about 9 μg/ml to about 10 μg/ml, from about 1 μg/ml to about 9 μg/ml, from about 2 μg/ml to about 9 μg/ml, from about 3 μg/ml to about 9 μg/ml, from about 4 μg/ml to about 9 μg/ml, from about 5 μg/ml to about 9 μg/ml, from about 6 μg/ml to about 9 μg/ml, from about 7 μg/ml to about 9 μg/ml, from about 8 μg/ml to about 9 μg/ml, from about 1 μg/ml to about 8 μg/ml, from about 2 μg/ml to about 8 μg/ml, from about 3 μg/ml to about 8 μg/ml, from about 4 μg/ml to about 8 μg/ml, from about 5 μg/ml to about 8 μg/ml, from about 6 μg/ml to about 8 μg/ml, from about 7 μg/ml to about 8 μg/ml, from about 1 μg/ml to about 7 μg/ml, from about 2 μg/ml to about 7 μg/ml, from about 3 μg/ml to about 7 μg/ml, from about 4 μg/ml to about 7 μg/ml, from about 5 μg/ml to about 7 μg/ml, from about 6 μg/ml to about 7 μg/ml, from about 1 μg/ml to about 6 μg/ml, from about 2 μg/ml to about 6 μg/ml, from about 3 μg/ml to about 6 μg/ml, from about 4 μg/ml to about 6 μg/ml, from about 5 μg/ml to about 6 μg/ml, from about 1 μg/ml to about 5 μg/ml, from about 2 μg/ml to about 5 μg/ml, from about 3 μg/ml to about 5 μg/ml, from about 4 μg/ml to about 5 μg/ml, from about 1 μg/ml to about 4 μg/ml, from about 2 μg/ml to about 4 μg/ml, from about 3 μg/ml to about 4 μg/ml, from about 1 μg/ml to about 3 μg/ml, from about 2 μg/ml to about 3 μg/ml, or from about 1 μg/ml to about 2 μg/ml). In other aspects, the agent capable of modulating the interaction of MOSPD2 and integrin 32 is an anti-MOSPD2 antibody, anti-integrin β2 antibody, or antigen binding fragment thereof, and is administered in the methods provided herein at an amount of from about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, or about 10 μg/ml.

In some aspects, the agent capable of modulating the interaction of CD63 and integrin β2 is an anti-CD63 antibody, anti-integrin β2 antibody, or antigen binding fragment thereof, and is administered in the methods provided herein at an amount of from about 1 μg/ml to about 10 μg/ml, or any range of values thereof (e.g., from about 2 μg/ml to about 10 μg/ml, from about 3 μg/ml to about 10 μg/ml, from about 4 μg/ml to about 10 μg/ml, from about 5 μg/ml to about 10 μg/ml, from about 6 μg/ml to about 10 μg/ml, from about 7 μg/ml to about 10 μg/ml, from about 8 μg/ml to about 10 μg/ml, from about 9 μg/ml to about 10 μg/ml, from about 1 μg/ml to about 9 μg/ml, from about 2 μg/ml to about 9 μg/ml, from about 3 μg/ml to about 9 μg/ml, from about 4 μg/ml to about 9 μg/ml, from about 5 μg/ml to about 9 μg/ml, from about 6 μg/ml to about 9 μg/ml, from about 7 μg/ml to about 9 μg/ml, from about 8 μg/ml to about 9 μg/ml, from about 1 μg/ml to about 8 μg/ml, from about 2 μg/ml to about 8 μg/ml, from about 3 μg/ml to about 8 μg/ml, from about 4 μg/ml to about 8 μg/ml, from about 5 μg/ml to about 8 μg/ml, from about 6 μg/ml to about 8 μg/ml, from about 7 μg/ml to about 8 μg/ml, from about 1 μg/ml to about 7 μg/ml, from about 2 μg/ml to about 7 μg/ml, from about 3 μg/ml to about 7 μg/ml, from about 4 μg/ml to about 7 μg/ml, from about 5 μg/ml to about 7 μg/ml, from about 6 μg/ml to about 7 μg/ml, from about 1 μg/ml to about 6 μg/ml, from about 2 μg/ml to about 6 μg/ml, from about 3 μg/ml to about 6 μg/ml, from about 4 μg/ml to about 6 μg/ml, from about 5 μg/ml to about 6 μg/ml, from about 1 μg/ml to about 5 μg/ml, from about 2 μg/ml to about 5 μg/ml, from about 3 μg/ml to about 5 μg/ml, from about 4 μg/ml to about 5 μg/ml, from about 1 μg/ml to about 4 μg/ml, from about 2 μg/ml to about 4 μg/ml, from about 3 μg/ml to about 4 μg/ml, from about 1 μg/ml to about 3 μg/ml, from about 2 μg/ml to about 3 μg/ml, or from about 1 μg/ml to about 2 μg/ml). In other aspects, the agent capable of modulating the interaction of CD63 and integrin β2 is an anti-CD63 antibody, anti-integrin β2 antibody, or antigen binding fragment thereof, and is administered in the methods provided herein at an amount of from about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, or about 10 μg/ml.

In some aspects, the agent capable of modulating the interaction of a complex of MOSPD2, integrin β2, and CD63 is an anti-MOSPD2 antibody, anti-integrin β2 antibody, and/or anti-CD63 antibody or antigen binding fragment thereof, and is administered in the methods provided herein at an amount of from about 1 μg/ml to about 10 μg/ml, or any range of values thereof (e.g., from about 2 μg/ml to about 10 μg/ml, from about 3 μg/ml to about 10 μg/ml, from about 4 μg/ml to about 10 μg/ml, from about 5 μg/ml to about 10 μg/ml, from about 6 μg/ml to about 10 μg/ml, from about 7 μg/ml to about 10 μg/ml, from about 8 μg/ml to about 10 μg/ml, from about 9 μg/ml to about 10 μg/ml, from about 1 μg/ml to about 9 μg/ml, from about 2 μg/ml to about 9 μg/ml, from about 3 μg/ml to about 9 μg/ml, from about 4 μg/ml to about 9 μg/ml, from about 5 μg/ml to about 9 μg/ml, from about 6 μg/ml to about 9 μg/ml, from about 7 μg/ml to about 9 μg/ml, from about 8 μg/ml to about 9 μg/ml, from about 1 μg/ml to about 8 μg/ml, from about 2 μg/ml to about 8 μg/ml, from about 3 μg/ml to about 8 μg/ml, from about 4 μg/ml to about 8 μg/ml, from about 5 μg/ml to about 8 μg/ml, from about 6 μg/ml to about 8 μg/ml, from about 7 μg/ml to about 8 μg/ml, from about 1 μg/ml to about 7 μg/ml, from about 2 μg/ml to about 7 μg/ml, from about 3 μg/ml to about 7 μg/ml, from about 4 μg/ml to about 7 μg/ml, from about 5 μg/ml to about 7 μg/ml, from about 6 μg/ml to about 7 μg/ml, from about 1 μg/ml to about 6 μg/ml, from about 2 μg/ml to about 6 μg/ml, from about 3 μg/ml to about 6 μg/ml, from about 4 μg/ml to about 6 μg/ml, from about 5 μg/ml to about 6 μg/ml, from about 1 μg/ml to about 5 μg/ml, from about 2 μg/ml to about 5 μg/ml, from about 3 μg/ml to about 5 μg/ml, from about 4 μg/ml to about 5 μg/ml, from about 1 μg/ml to about 4 μg/ml, from about 2 μg/ml to about 4 μg/ml, from about 3 μg/ml to about 4 μg/ml, from about 1 μg/ml to about 3 μg/ml, from about 2 μg/ml to about 3 μg/ml, or from about 1 μg/ml to about 2 μg/ml). In other aspects, the agent capable of modulating the interaction of a complex of MOSPD2, integrin β2, and CD63 is an anti-MOSPD2 antibody, anti-integrin β2 antibody, and/or anti-CD63 antibody or antigen binding fragment thereof, and is administered in the methods provided herein at an amount of from about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, or about 10 μg/ml.

In some aspects, the agent capable of modulating the interaction of MOSPD2 and CD63 is an anti-MOSPD2 antibody, anti-CD63 antibody, or antigen binding fragment thereof, and is administered in the methods provided herein at an amount of from about 10 mg/kg to about 40 mg/kg, or any range of values thereof (e.g., from about 15 mg/kg to about 40 mg/kg, from about 20 mg/kg to about 40 mg/kg, from about 25 mg/kg to about 40 mg/kg, from about 30 mg/kg to about 40 mg/kg, from about 35 mg/kg to about 40 mg/kg, from about 10 mg/kg to about 35 mg/kg, from about 15 mg/kg to about 35 mg/kg, from about 20 mg/kg to about 35 mg/kg, from about 25 mg/kg to about 35 mg/kg, from about 30 mg/kg to about 35 mg/kg, from about 10 mg/kg to about 30 mg/kg, from about 15 mg/kg to about 30 mg/kg, from about 20 mg/kg to about 30 mg/kg, from about 25 mg/kg to about 30 mg/kg, from about 10 mg/kg to about 25 mg/kg, from about 15 mg/kg to about 25 mg/kg, from about 20 mg/kg to about 25 mg/kg, from about 10 mg/kg to about 20 mg/kg, from about 15 mg/kg to about 20 mg/kg, or from about 10 mg/kg to about 15 mg/kg). In some aspects, the agent capable of modulating the interaction of MOSPD2 and CD63 is an anti-MOSPD2 antibody, anti-CD63 antibody, or antigen binding fragment thereof, and is administered in the methods provided herein at an amount of about 10 mg/kg, about 15 mg/kg, about 20 mg/kg, about 25 mg/kg, about 30 mg/kg, about 35 mg/kg, or about 40 mg/kg.

In some aspects, the agent capable of modulating the interaction of MOSPD2 and integrin β2 is an anti-MOSPD2 antibody, anti-integrin β2 antibody, or antigen binding fragment thereof, and is administered in the methods provided herein at an amount of from about 10 mg/kg to about 40 mg/kg, or any range of values thereof (e.g., from about 15 mg/kg to about 40 mg/kg, from about 20 mg/kg to about 40 mg/kg, from about 25 mg/kg to about 40 mg/kg, from about 30 mg/kg to about 40 mg/kg, from about 35 mg/kg to about 40 mg/kg, from about 10 mg/kg to about 35 mg/kg, from about 15 mg/kg to about 35 mg/kg, from about 20 mg/kg to about 35 mg/kg, from about 25 mg/kg to about 35 mg/kg, from about 30 mg/kg to about 35 mg/kg, from about 10 mg/kg to about 30 mg/kg, from about 15 mg/kg to about 30 mg/kg, from about 20 mg/kg to about 30 mg/kg, from about 25 mg/kg to about 30 mg/kg, from about 10 mg/kg to about 25 mg/kg, from about 15 mg/kg to about 25 mg/kg, from about 20 mg/kg to about 25 mg/kg, from about 10 mg/kg to about 20 mg/kg, from about 15 mg/kg to about 20 mg/kg, or from about 10 mg/kg to about 15 mg/kg). In some aspects, the agent capable of modulating the interaction of MOSPD2 and integrin β2 is an anti-MOSPD2 antibody, anti-integrin β2 antibody, or antigen binding fragment thereof, and is administered in the methods provided herein at an amount of about 10 mg/kg, about 15 mg/kg, about 20 mg/kg, about 25 mg/kg, about 30 mg/kg, about 35 mg/kg, or about 40 mg/kg.

In some aspects, the agent capable of modulating the interaction of CD63 and integrin β2 is an anti-CD63 antibody, anti-integrin β2 antibody, or antigen binding fragment thereof, and is administered in the methods provided herein at an amount of from about 10 mg/kg to about 40 mg/kg, or any range of values thereof (e.g., from about 15 mg/kg to about 40 mg/kg, from about 20 mg/kg to about 40 mg/kg, from about 25 mg/kg to about 40 mg/kg, from about 30 mg/kg to about 40 mg/kg, from about 35 mg/kg to about 40 mg/kg, from about 10 mg/kg to about 35 mg/kg, from about 15 mg/kg to about 35 mg/kg, from about 20 mg/kg to about 35 mg/kg, from about 25 mg/kg to about 35 mg/kg, from about 30 mg/kg to about 35 mg/kg, from about 10 mg/kg to about 30 mg/kg, from about 15 mg/kg to about 30 mg/kg, from about 20 mg/kg to about 30 mg/kg, from about 25 mg/kg to about 30 mg/kg, from about 10 mg/kg to about 25 mg/kg, from about 15 mg/kg to about 25 mg/kg, from about 20 mg/kg to about 25 mg/kg, from about 10 mg/kg to about 20 mg/kg, from about 15 mg/kg to about 20 mg/kg, or from about 10 mg/kg to about 15 mg/kg). In some aspects, the agent capable of modulating the interaction of CD63 and integrin β2 is an anti-CD63 antibody, anti-integrin β2 antibody, or antigen binding fragment thereof, and is administered in the methods provided herein at an amount of about 10 mg/kg, about 15 mg/kg, about 20 mg/kg, about 25 mg/kg, about 30 mg/kg, about 35 mg/kg, or about 40 mg/kg.

In some aspects, the agent capable of modulating the interaction of a complex of MOSPD2, integrin β2, and CD63 is an anti-MOSPD2 antibody, anti-integrin β2 antibody, and/or anti-CD63 antibody or antigen binding fragment thereof, and is administered in the methods provided herein at an amount of from about 10 mg/kg to about 40 mg/kg, or any range of values thereof (e.g., from about 15 mg/kg to about 40 mg/kg, from about 20 mg/kg to about 40 mg/kg, from about 25 mg/kg to about 40 mg/kg, from about 30 mg/kg to about 40 mg/kg, from about 35 mg/kg to about 40 mg/kg, from about 10 mg/kg to about 35 mg/kg, from about 15 mg/kg to about 35 mg/kg, from about 20 mg/kg to about 35 mg/kg, from about 25 mg/kg to about 35 mg/kg, from about 30 mg/kg to about 35 mg/kg, from about 10 mg/kg to about 30 mg/kg, from about 15 mg/kg to about 30 mg/kg, from about 20 mg/kg to about 30 mg/kg, from about 25 mg/kg to about 30 mg/kg, from about 10 mg/kg to about 25 mg/kg, from about 15 mg/kg to about 25 mg/kg, from about 20 mg/kg to about 25 mg/kg, from about 10 mg/kg to about 20 mg/kg, from about 15 mg/kg to about 20 mg/kg, or from about 10 mg/kg to about 15 mg/kg). In some aspects, the agent capable of modulating the interaction of a complex of MOSPD2, integrin β2, and CD63 is an anti-MOSPD2 antibody, anti-integrin β2 antibody, and/or anti-CD63 antibody or antigen binding fragment thereof, and is administered in the methods provided herein at an amount of about 10 mg/kg, about 15 mg/kg, about 20 mg/kg, about 25 mg/kg, about 30 mg/kg, about 35 mg/kg, or about 40 mg/kg.

In some aspects of any of the methods provided herein, the subject is a human. In some aspects, the subject is a mammal. In some aspects, the subject is a veterinary animal (e.g., a dog, cat, bird, mouse, horse, sheep, cow, goat, or the like).

### EXAMPLES

Reference is now made to the following examples, which together with the above descriptions illustrate some aspects of the disclosure in a non-limiting fashion.

### Example 1

### CD63 and MOSPD2 Have a Similar Role in the Migration of Myeloid Cells During Inflammation

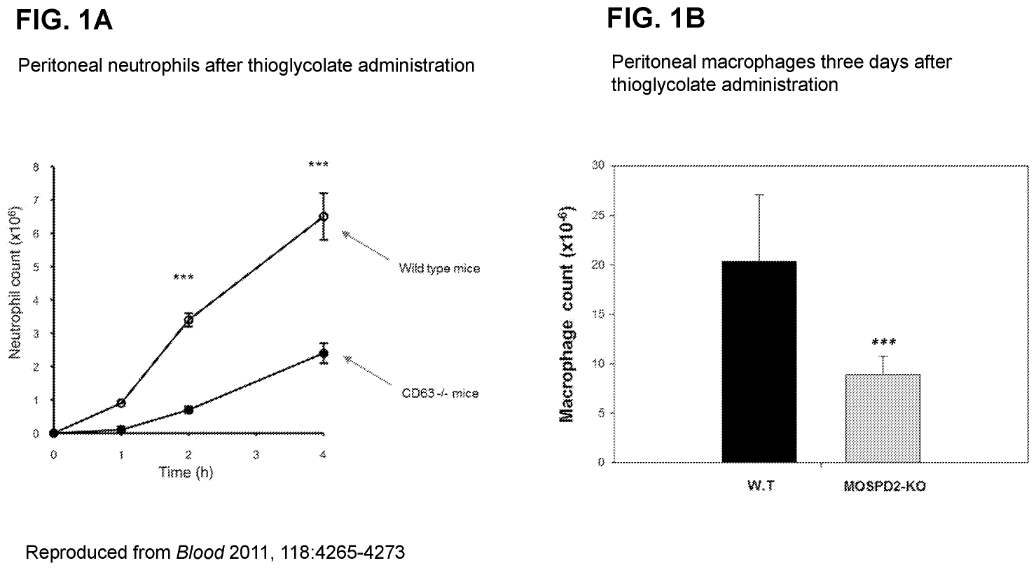

FIG. 1A (reproduced from Blood 2011, 118:4265-4273) shows the number of neutrophils counted at 1, 2, 3 and 4 hours after the induction of inflammation with thioglycolate in wild type mice and CD63 knockout (−/−) mice. The number of neutrophils was significantly reduced in CD63 knockout mice compared to wild type mice, therefore showing that CD63 is involved in the migration of myeloid cells during inflammation.

MOSPD2-deficient and wild type mice (n=9/group) were injected with thioglycolate (1 ml) into the peritoneal cavity. After three days, mice were sacrificed, and the peritoneal cavity was rinsed 3-4 times with 4 ml ice-cold Roswell Park Memorial Institute (RPMI) medium without phenol red until a clear solution was observed. Cell suspension was collected into a sterile 50 ml plastic tube. Cells were spun down, resuspended in phosphate-buffered saline (PBS), and the number of macrophages was counted.

FIG. 1B shows the number of macrophages counted 3 days after the induction of inflammation with thioglycolate in wild type (W.T) mice and MOSPD2 knock out (MOSPD2-KO) mice. The number of macrophages was significantly reduced in MOSPD2 knock out mice compared to wild type mice, therefore demonstrating that MOSPD2 is involved in the migration of myeloid cells during inflammation. Statistical significance of *** p<0.001 by t-test.

Furthermore, a comparison of the results in FIG. 1A and FIG. 1B demonstrates that MOSPD2 and CD63 have a similar effect on the migration of myeloid cells during inflammation.

### Example 2

### CD63 Plays a Role in Monocyte Migration

To establish a role for CD63 in monocyte motility, we first silenced its expression. For that, Clustered Regularly Interspaced Short Palindromic Repeats (CRISPR) non-target control (CRISPR-Control) or CD63-CRISPR lentiviral particles were added to THP-1 monocytic cells (2×10⁶ in 2 ml). Cells were spun for 60 min at 2000 rpm at room temperature in the presence of 8 μg/ml polybrene and then seeded in a 6-well plate. After 72 hr, fresh medium containing puromycin (4 μg/ml) was added for the selection of transduced cells. Single-cell cloning was performed on CRISPR-transduced cells to isolate cells with silenced MOSPD2 protein expression. Cells were stained with PE-conjugated anti-CD63 antibody (CD63-CRISPR) or PE-isotype control antibody.

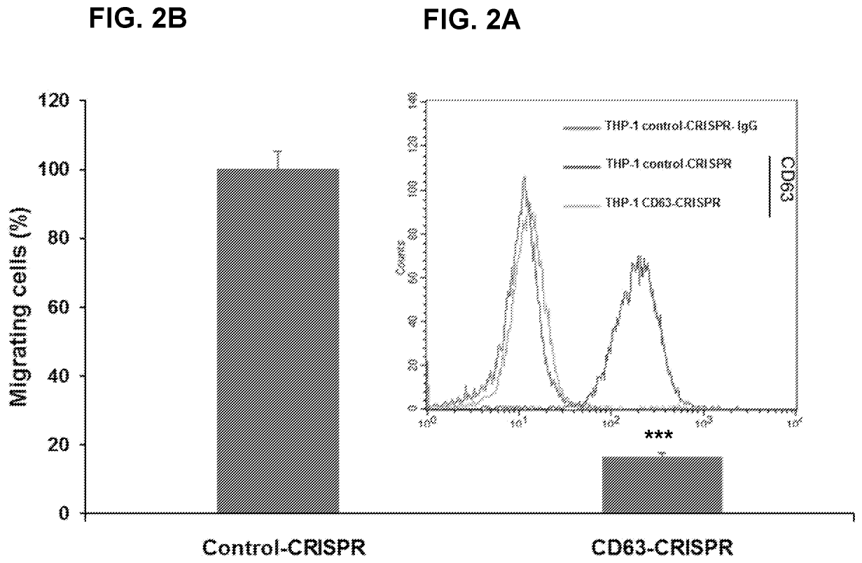

FIG. 2A shows staining of a successfully CD63-silenced clone (THP-1 CD63-CRISPR) compared with control-silenced THP-1 cells (THP-1 control-CRISPR and THP-1 CD63-CRISPR) detected by flow cytometry.

Then, control and CD63 silenced cells were tested for their ability to migrate in response to chemokines. MCP-1 and SDF-1 (100 ng/ml each) were dissolved in 0.5% FBS/RPMI-1640 and placed in the lower chamber of a QCM 24-well migration assay plate (5 μm pores) (Corning-Costar, Corning, NY). Cells (3×10⁵) were seeded in the upper chamber, followed by incubation for 3 hr, after which the number of cells that migrated to the lower compartment was determined by flow cytometry.

FIG. 2B shows migration of CD63-silenced THP-1 cells (CD63-CRISPR) relative to control THP-1 cells (Control-CRISPR) towards SDF-1+MCP-1. Samples were run in triplicates. Results are shown as mean±SD. Statistical significance of *** p<0.001 by t-test.

These data show that inhibition of CD63 with CRISPR resulted in significantly reduced monocyte migration compared to control cells. These data demonstrate that CD63 plays a role in monocyte migration.

### Example 3

### MOSPD2 Regulates Monocyte Adhesion

MOSPD2 was silenced in THP-1 cells using CRISPR non-target control (CRISPR-Control) or CRISPR-MOSPD2 lentiviral particles, as explained in Example 2. Two different clones of CRISPR-MOSPD2 cells were analyzed, MOSPD2 M1 and MOSPD2 M2.

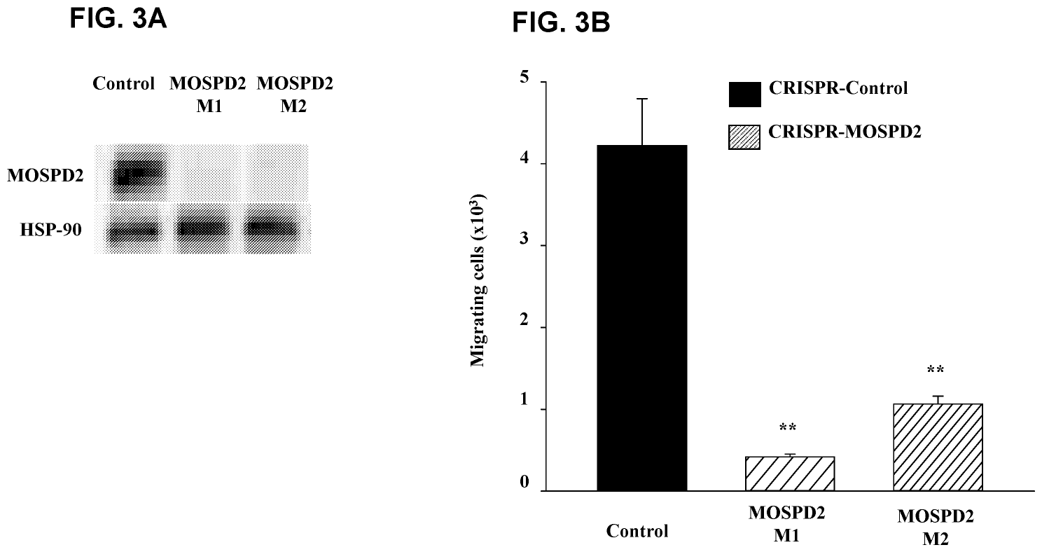

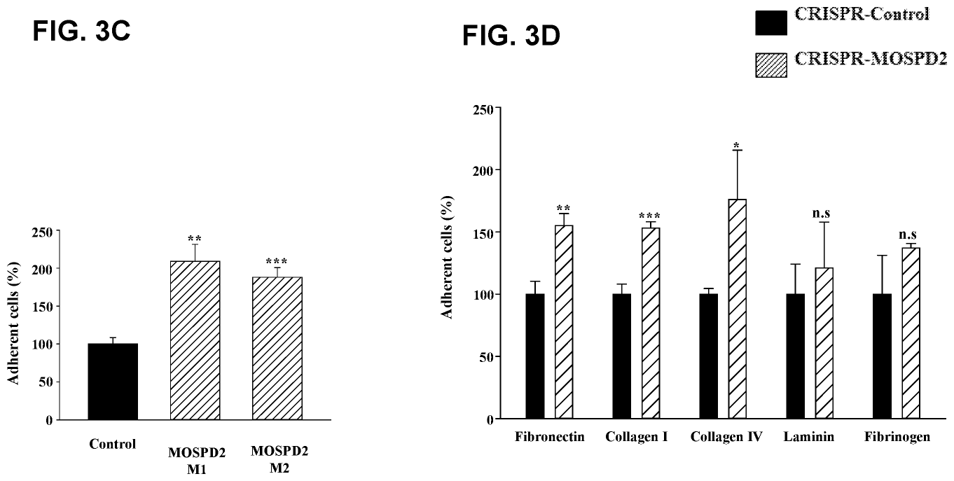

Silencing of MOSPD2 expression in MOSPD2 M1 and MOSPD2 M2 was confirmed by western blot. In brief, CRISPR-Control, CRISPR-MOSPD2 M1 and CRISPR-MOSPD2 M2 cell cultures were lysed using 1% NP-40 lysis buffer containing protease inhibitors, followed by 30 min incubation on ice and 25 min centrifugation at maximum speed. Samples were run on a SDS-polyacrylamide gel electrophoresis (SDS-PAGE) gel and blotted with anti-MOSPD2 monoclonal antibody or anti-HSP-90 antibody as indicated in FIG. 3A.

FIG. 3A shows that MOSPD2 protein expression was silenced in CRISPR-MOSPD2 M1 and CRISPR-MOSPD2 M2, as anti-MOSPD2 antibody did not bind to CRISPR-MOSPD2 M1 and CRISPR-MOSPD2 M2 lysates, but did bind to control lysate.

Next, control and MOSPD2-silenced cells were tested for their ability to migrate in response to chemokines. Regulated upon Activation, Normal T Cell Expressed and Presumably Secreted (RANTES) and Monocyte Chemotactic Protein-1 (MCP-1) (100 ng/ml each) were dissolved in 0.5% FBS/RPMI-1640 and placed in the lower chamber of a QCM 24-well migration assay plate (5 μm pores) (Corning-Costar, Corning, NY). Cells (3×10⁵) were seeded in the upper chamber, followed by incubation for 3 hr, after which the number of cells that migrated to the lower compartment was determined by flow cytometry. Samples were run in triplicate.

Results are shown in FIG. 3B as mean number of migrating control and MOSPD2-silenced cells ±standard deviation (SD). These results show that CRISPR-MOSPD2 M1 and CRISPR-MOSPD2 M2 had significantly less monocyte migration in response to chemokines compared to control cells.

Next, we explored how silencing of MOSPD2 affected monocyte binding to the extracellular matrix (ECM). For that, CRISPR-Control, CRISPR-MOSPD2 M1 and CRISPR-MOSPD2 M2 cells (10⁶/ml) were incubated in starvation medium for 40 min. Then, cells (10⁶/well) were loaded onto Collagen IV-coated plates for 90 minutes at 37° C., followed by 3 washes with PBS. 200 μl of staining solution was then added for 10 min, washed 4 times with DDW, and left to air dry. 200 μl of extraction solution was added to each well for 10 min on an orbital shaker. 150 μl from each extracted sample was transferred to a 96-well microtiter plate and the plate was read at OD 560 nm in a plate reader. Samples were run in triplicate.

Results are shown in FIG. 3C as the mean percentage of adherent CRISPR-MOSPD2 M1 and CRISPR-MOSPD2 M2 cells ±SD, relative to adherent CRISPR-Control cells. These results show that CRISPR-MOSPD2 M1 and CRISPR-MOSPD2 M2 had significantly greater adhesion to Collagen IV compared to CRISPR-Control cells.

Adhesion to other ECM proteins was also tested. FIG. 3D shows the mean percentage of adherent CRISPR-MOSPD2 M1 cells ±SD, relative to adherent CRISPR-Control cells to Fibronectin, Collagen I, Collagen IV, Laminin and Fibrinogen. These results show that CRISPR-MOSPD2 M1 and CRISPR-MOSPD2 M2 cells had greater adhesion to other ECM proteins compared to CRISPR-Control cells.

### Example 4

### MOSPD2 and Integrin β2 Demonstrate Differential Regulation on Monocyte Adhesion

Integrin β2 was silenced in THP-1 cells using CRISPR non-target control (CRISPR-Control) or CRISPR-Integrin 2 lentiviral particles, as explained in Example 2. Two different clones of CRISPR-Integrin β2 cells were analyzed, CRISPR-Integrin β2 I1 and CRISPR-Integrin β2 12.

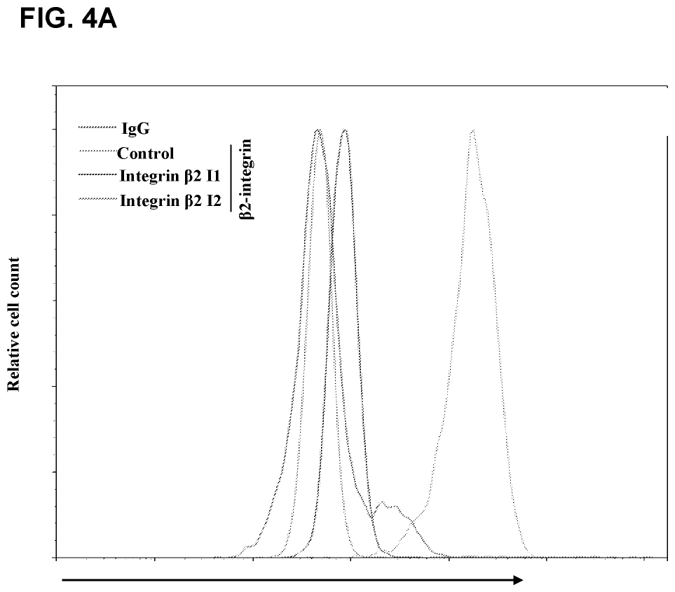

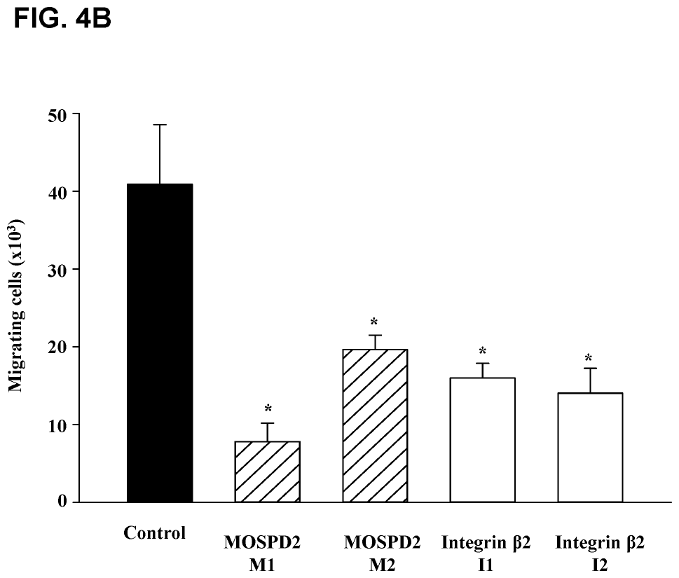

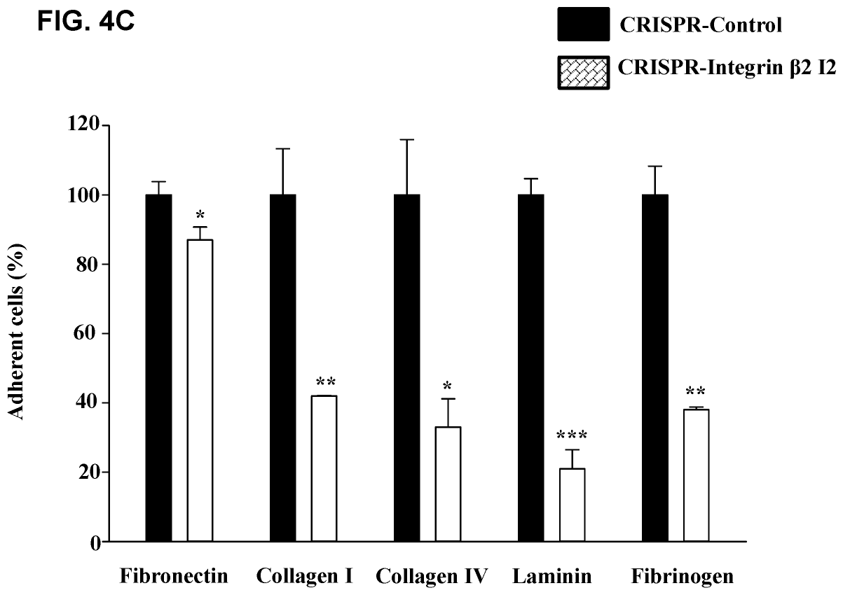

FIG. 4A shows staining of CRISPR-Integrin β2 I1 and CRISPR-Integrin β2 I2 cells and control THP-1 cells (IgG and CRISPR-Control cells) detected by flow cytometry. CRISPR-Control cells were also stained with an PE-labeled isotype-matched antibody

Next, control, CRISPR-MOSPD2 and CRISPR-Integrin β2 cells were tested for their ability to migrate in response to RANTES and MCP-1 (100 ng/ml each), as explained in Example 3. Results are shown in FIG. 4B as the mean number of migrating control, MOSPD2-silenced or Integrin β2-silenced cells ±standard deviation (SD). These results show that CRISPR-MOSPD2 and CRISPR-Integrin β2 had significantly less monocyte migration in response to RANTES and MCP-1 compared to control cells.

The effect of Integrin β2 silencing on binding to ECM proteins was also tested, as explained in Example 3. Results are shown in FIG. 4C as the mean percentage of adherent CRISPR-Integrin β2 I2 cells ±SD, relative to adherent CRISPR-Control cells. These results show that CRISPR-Integrin β2 cells had significantly less adhesion to Fibronectin, Collagen I, Collagen IV, Laminin, and Fibrinogen compared to CRISPR-Control cells. This result is in contrast to the result in FIG. 3D, where MOSPD2 silencing resulted in greater adhesion to ECM proteins.

### Example 5

### MOSPD2 Binds CD63

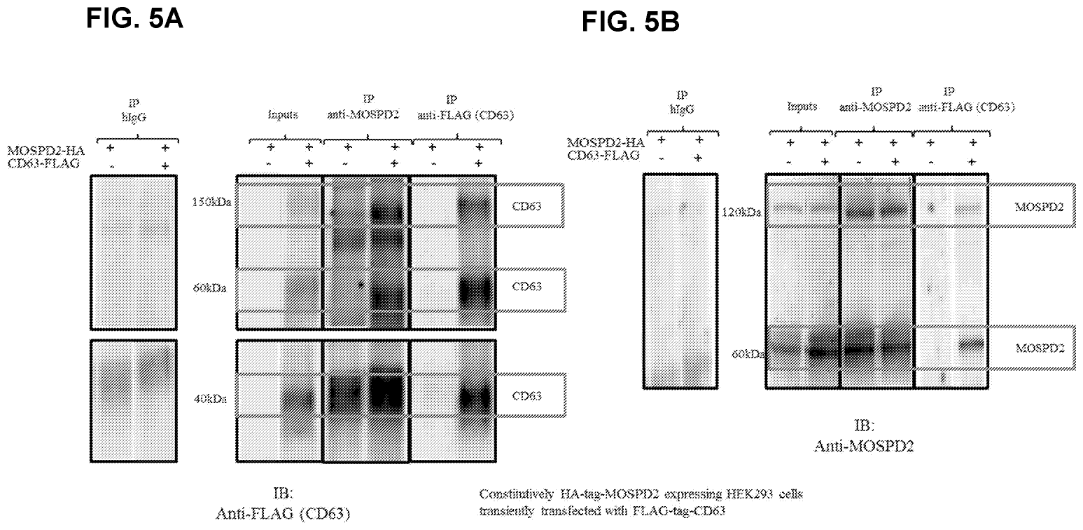

To gain additional evidence for CD63-MOSPD2 interaction, constitutively expressing HA-tagged MOSPD2 expressing HEK293 cells were un-transfected or transiently transfected with FLAG-tagged CD63. Cells were then lysed using 1% 98-Brij lysis buffer containing protease inhibitors, followed by 60 min incubation on ice and 25 min centrifugation at maximum speed. Samples were incubated in a rotator overnight at 4° C. with a human anti-MOSPD2 monoclonal antibody (mAb), anti-FLAG antibody or IgG control antibody. Protein A/G was added for 2 hr at 4° C. Protein elution was performed with loading buffer containing Tris, sodium dodecyl sulfate (SDS), and glycerol for 10 min at room temperature (RT). Proteins eluted were then run on SDS-polyacrylamide gel electrophoresis (SDS-PAGE) gel and blotted as indicated in FIG. 5A and FIG. 5B.

The data in FIG. 5A show that precipitation (IP) with anti-MOSPD2 mAb and blotting (IB) with anti-FLAG antibody results in the detection of CD63, whereas staining for CD63 could not be detected following precipitation with an IgG control antibody or with anti-MOSPD2 mAb on un-transfected cells.

The results in FIG. 5B show that CD63 coprecipitates with MOSPD2. MOSPD2 could not be detected when precipitation was performed with IgG control antibody or with anti-FLAG antibody on un-transfected cells. Taken together, these results indicate that MOSPD2 binds CD63.

### Example 6

### MOSPD2 Binds Integrin β2

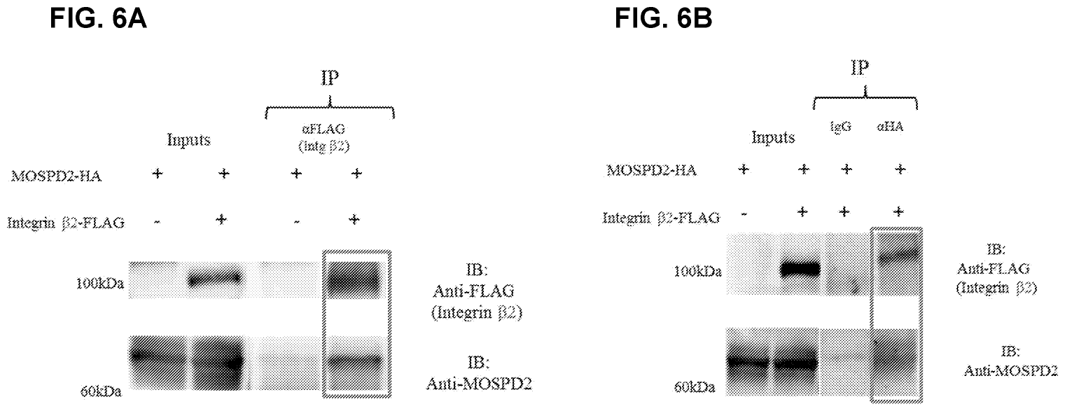

Constitutively expressing HA-tagged MOSPD2 expressing HEK293 cells were un-transfected or transiently transfected with FLAG-tagged integrin β2. Cells were then lysed using 1% 98-Brij lysis buffer containing protease inhibitors, followed by 60 min incubation on ice and 25 min centrifugation at maximum speed. Samples were incubated in a rotator overnight at 4° C. with a human anti-MOSPD2 mAb, anti-FLAG antibody or IgG control antibody. Protein A/G was added for 2 hr at 4° C. Protein elution was performed with loading buffer containing Tris, SDS, and glycerol for 10 min at room temperature (RT). Proteins eluted were then run on sodium dodecyl sulphate-polyacrylamide gel electrophoresis (SDS-PAGE) gel and blotted as indicated in FIG. 6A and FIG. 6B.

The data in FIG. 6A show that precipitation (IP) with anti-FLAG antibody and blotting (IB) with anti-MOSPD2 mAb results in detection of MOSPD2, which could not be detected upon precipitation with anti-FLAG antibody on un-transfected cells.

To gain additional evidence for MOSPD2-integrin β2 interaction, cell lysates were also precipitated with anti-hemagglutinin (anti-HA) antibody or IgG control antibody and blotted with anti-FLAG antibody. The results in FIG. 6B affirm that integrin β2 coprecipitates with MOSPD2.

Taken together, these results demonstrate that MOSPD2 binds integrin β2.

### Example 7

### Anti-MOSPD2 Antibody, Anti-Integrin β2 Antibodies, and Anti-CD63 Antibody Inhibit Monocyte Migration

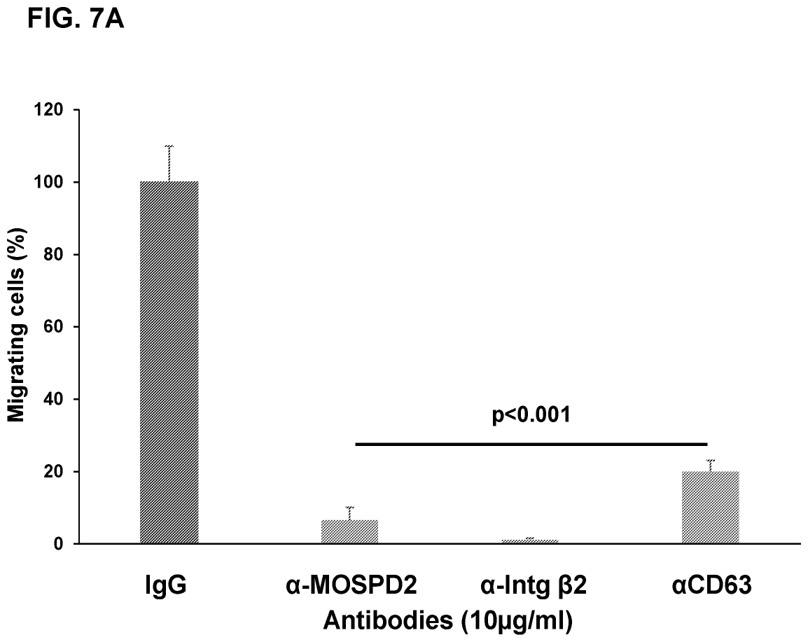

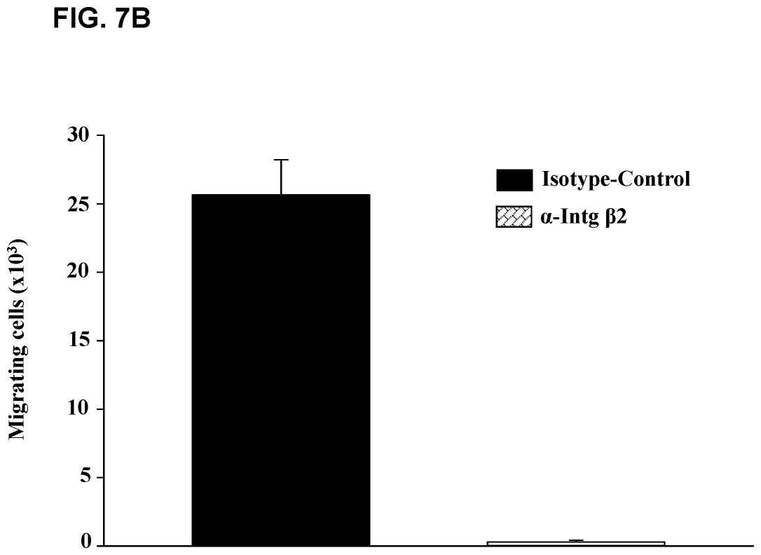

To support the interaction between MOSPD2, CD63 and integrin β2, we compared their effect on monocyte migration using specific mAbs. For that, CD14 monocytes were isolated from blood peripheral blood mononuclear cells (PBMCs) from healthy donors with human CD14 microbeads. MCP-1 and SDF-1 (100 ng/ml each, PeproTech, Israel) were dissolved in 0.5% FBS/RPMI-1640 and placed in the lower chamber of a QCM 24-well migration assay plate (5 μm pores) (Corning-Costar, Corning, NY). Cells (3×10⁵) pre-incubated with 10 μg/ml of the antibodies indicated in FIG. 7A for 30 min were seeded in the upper chamber, followed by incubation for 3 hr, after which the number of cells that migrated towards MCP-1 and SDF-1 in the lower compartment was determined by flow cytometry.

FIG. 7A shows the percentage of migrating monocytes following treatment with anti-MOSPD2 mAb (αMOSPD2), anti-integrin β2 mAb (α-Intg β2), anti-CD63 mAb (αCD63), or control antibody (IgG). The results depicted in FIG. 7A show that anti-MOSPD2 mAb, anti-integrin β2 mAb and anti-CD63 mAb display significant (p<0.001) and similar effect on inhibiting monocyte migration when compared with IgG control antibody. Samples were run in triplicates. Results are shown as mean±SD.

FIG. 7B provides additional data showing the mean number of migrating CD14+ monocytes ±SD following treatment with a control or polyclonal anti-integrin β2 antibody (α-Intg β2) toward RANTES and MCP-1 (100 ng/mL each). Samples were run in triplicate.

These data demonstrate that treatment with anti-MOSPD2 antibody, anti-integrin β2 antibody, or anti-CD63 antibody resulted in significantly decreased monocyte migration.

### Example 8

### Anti-MOSPD2 Antibody Anti-Integrin β2 Antibody, and Anti-CD63 Antibody Promote Monocyte Adhesion to Fibronectin

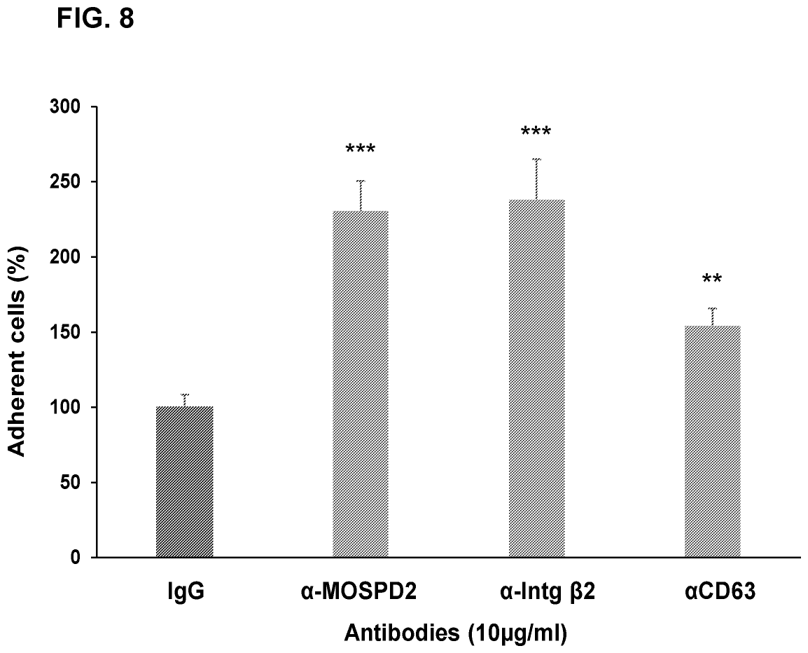

We explored how targeting a complex of MOSPD2, integrin β2 and CD63 will affect monocyte binding to the extracellular matrix (ECM). For that, CD14 monocytes (10⁶/ml) were incubated in starvation medium in the presence of the antibodies indicated in FIG. 7A (10 μg/ml) for 40 min. Then, cells (10⁶/well) were loaded onto fibronectin pre-coated plates (CELL BIOLABS) for 90 minutes at 37° C., followed by 3 washes with PBS. Then, 200 μl of staining solution was added for 10 min, washed 4 times with DDW and left to air dry. Next, 200 μl of extraction solution was added to each well for 10 min on an orbital shaker. 150 μl from each extracted sample was transferred to a 96-well microtiter plate and the plate was read at OD 560 nm in a plate reader. The results in FIG. 8 show that treatment with anti-CD63 antibody and more profoundly with anti-MOSPD2 or anti-integrin β2 antibodies significantly promotes adhesion of monocytes to fibronectin. Adhesion relative to IgG control antibody is shown. Samples were run in triplicates. Results are shown as mean±SD. Statistical significance of **p<0.01, ***p<0.001 by t-test.

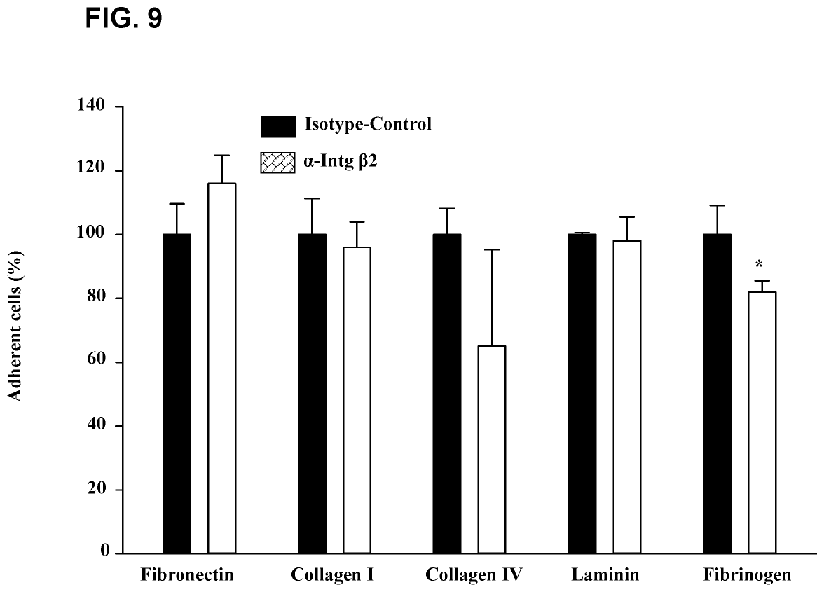

FIG. 9 provides additional data showing the mean percentage of adherent cells SD following treatment with polyclonal anti-integrin β2 antibody (α-Intg β2) relative to adherent cells following treatment with control antibody to different ECM proteins. Samples were run in triplicate. *p<0.05 by t-test. These results suggest that the effect different antibodies targeting integrin-β2 impart on monocyte adhesion may depend on the ECM in test.

### Example 9

### Anti-MOSPD2 Antibody-Driven Increased Adhesion of CD14+ Primary Monocytes to Fibronectin is Dose-Dependent

We explored whether anti-MOSPD2 mAb promotes adhesion of monocytes to fibronectin in a dose dependent manner, CD14 monocytes (10⁶/ml) were incubated in starvation medium in the presence of the anti-MOSPD2 mAb at the concentrations indicated in FIG. 7 for 40 min. Then, cells (10⁶/well) were loaded onto fibronectin pre-coated plates (CELL BIOLABS) for 90 minutes at 37° C., followed by 3 washes with PBS. Then, 200 μl of staining solution was added for 10 min, washed 4 times with DDW and left to air dry. Next, 200 μl of extraction solution was added to each well for 10 min on an orbital shaker. 150 μl from each extracted sample was transferred to a 96-well microtiter plate and the plate was read at OD 560 nm in a plate reader.

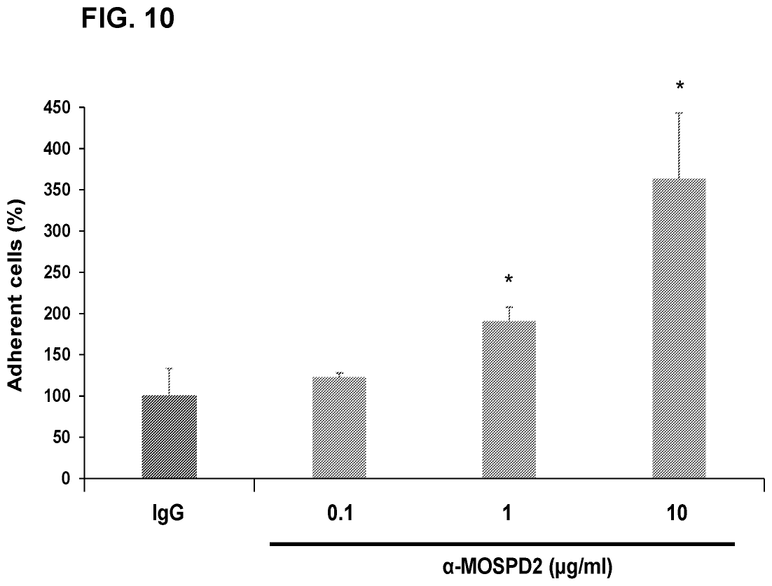

The effect by anti-MOSPD2 mAb was dose dependent (FIG. 10). Adhesion relative to IgG control antibody is shown. Samples were run in triplicate. Results are shown as mean±SD. Statistical significance of *p<0.05 by t-test.

### Example 10

### Anti-MOSPD2 Antibody Increases Adhesion of CD14 Primary Monocytes to ECM Ligands and Adhesion Molecules

We explored how targeting MOSPD2 will affect monocyte binding to collagen. For that, CD14 monocytes (10⁶/ml) were incubated in starvation medium in the presence of the anti-MOSPD2 mAb (10 μg/ml) for 40 min. Then, cells (10⁶/well) were loaded onto collagen IV pre-coated plates (CELL BIOLABS) for 90 minutes at 37° C., followed by 3 washes with PBS. Then, 200 μl of staining solution was added for 10 min, washed 4 times with DDW and left to air dry. Next, 200 μl of extraction solution was added to each well for 10 min on an orbital shaker. 150 μl from each extracted sample was transferred to a 96-well microtiter plate and the plate was read at OD 560 nm in a plate reader.

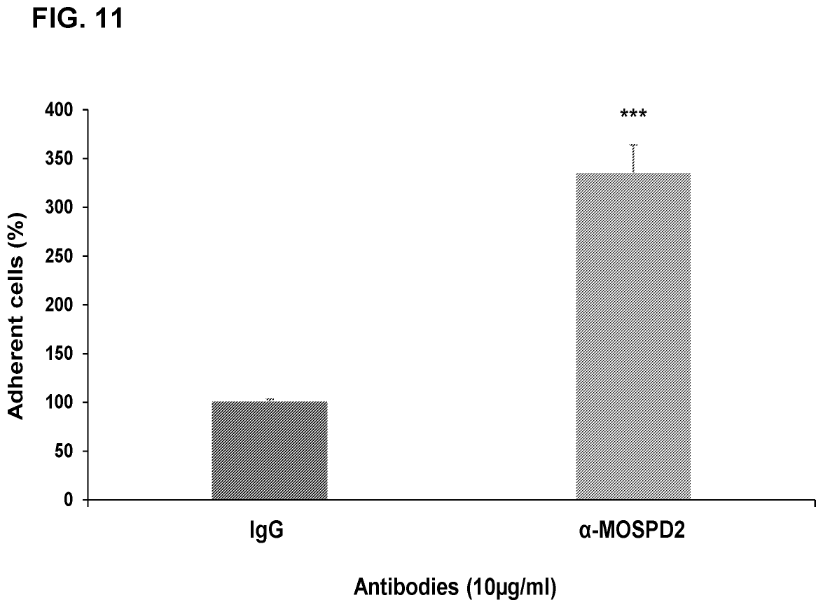

The effect by anti-MOSPD2 mAb was not restricted to one type of ECM component but could also be demonstrated for the ECM protein collagen IV (FIG. 11). FIG. 11 shows adhesion to collagen IV coated plates of freshly isolated human primary monocytes incubated with 10 μg/ml of the indicated antibodies. Adhesion relative to IgG control antibody is shown. Samples were run in triplicate. Results are shown as mean SD. Statistical significance of ***p<0.001 by t-test.

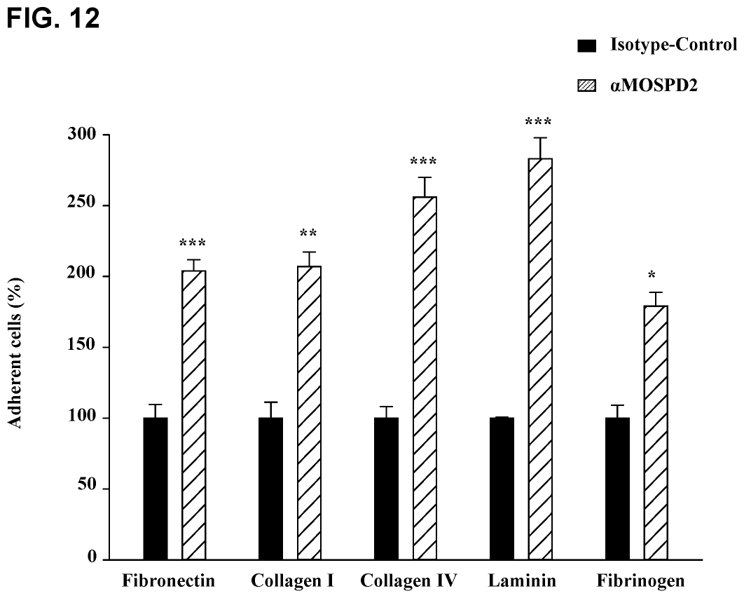

FIG. 12 provides additional data showing adhesion of CD14+ monocytes treated with isotype control or anti-MOSPD2 antibody (αMOSPD2, 10 μg/mL) to a plate coated with different extracellular matrix ligands. The percentage of adherent cells treated with αMOSPD2 relative to adherent control cells is presented. Samples were run in triplicate. Results are shown in mean±SD. *p<0.05, **p<0.01, ***p<0.001 by t-test.

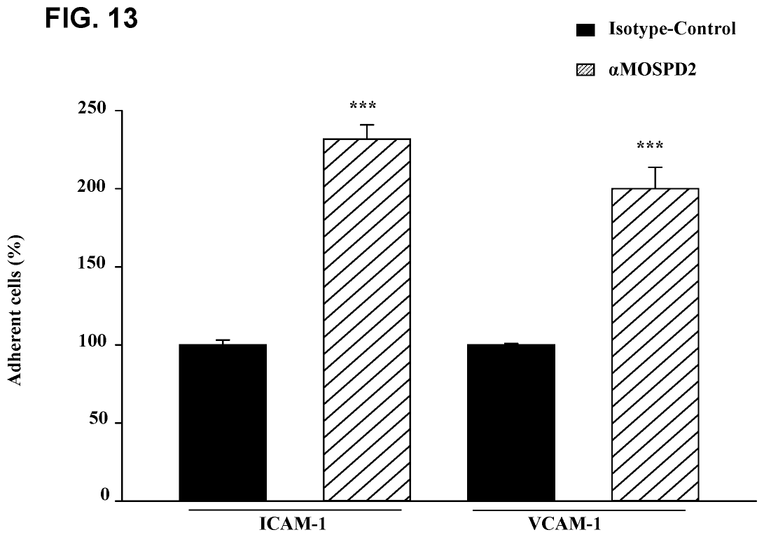

FIG. 13 provides additional data showing adhesion of CD14+ monocytes treated with isotype control or anti-MOSPD2 antibody (αMOSPD2, 10 μg/mL) to a plate coated with ICAM-1 or VCAM-1. The percentage of adherent cells treated with αMOSPD2 relative to adherent control cells is presented. Samples were run in triplicate. Results are shown in mean±SD. ***p<0.001 by t-test.

These data show that anti-MOSPD2 antibody treatment increased adhesion of CD14+ monocytes to different ECM proteins and adhesion molecules.

### Example 11

### Anti-MOSPD2 Antibody Induces Signaling Events Associated with Integrin Activation

Activation of cells via integrins induces a chain of phosphorylation events that promotes cell adhesion and migration. To better understand the mechanism through which anti-MOSPD2 mAb increases binding of monocytes to adhesion molecules and ECM, monocytes (10⁶/well) were seeded on plates in starvation medium for 40 min. IgG control antibody or anti-MOSPD2 mAb (10 μg/ml) was added for the indicated times. Wells were then washed with PBS and lysed in lysis buffer containing protease and phosphatase inhibitors. Samples were loaded on SDS-PAGE gel and blotted for detecting the indicated phosphorylated proteins.

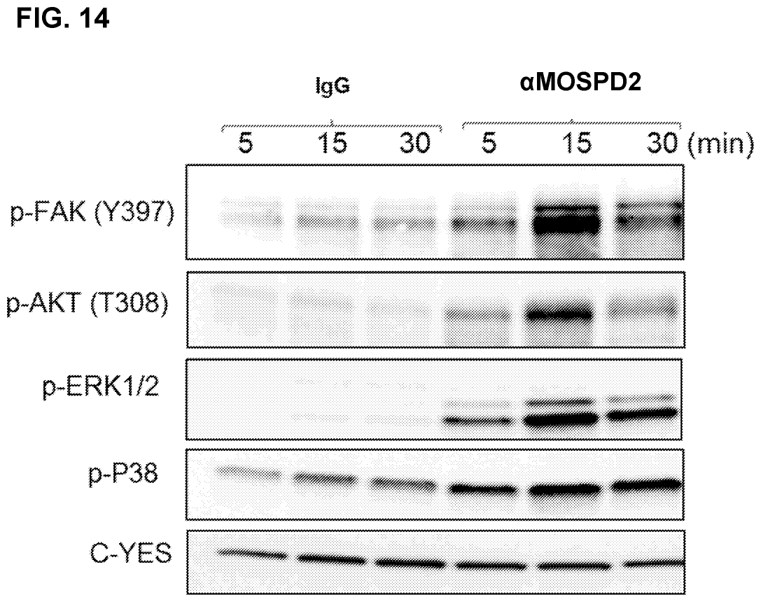

Freshly isolated human primary monocytes were seeded and incubated with 10 μg/ml of IgG control or anti-MOSPD2 mAb for the indicated time points. C-YES was used as loading control. The results in FIG. 14 show that in the presence of anti-MOSPD2 mAb, phosphorylation of proteins that are associated with monocyte adhesion and migration are markedly induced.

### Example 12

### Effect of Anti-MOSPD2 Antibody, Natalizumab (Anti-α4β1 Antibody) and Vedolizumab (Anti-α4β7 Antibody) on Primary Monocyte Migration

To compare the effect of anti-MOSPD2 (αMOSPD2) to Natalizumab (anti-α41 antibody) and Vedolizumab (anti-α4β7 antibody) on monocyte migration, MCP-1 and SDF-1 (100 ng/ml each, PeproTech, Israel) were dissolved in 0.5% FBS/RPMI-1640 and placed in the lower chamber of a QCM 24-well migration assay plate (5 μm pores) (Corning-Costar, Corning, NY). Human primary monocytes (3×10⁵) isolated with CD14 microbeads were pre-incubated with the indicated antibodies for 30 min and seeded in the upper chamber, followed by incubation for 3 hr, after which the number of cells that migrated to the lower compartment was determined by flow cytometry.

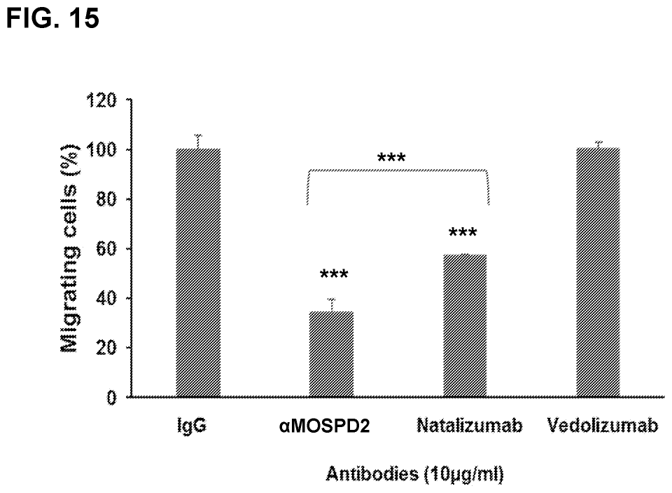

FIG. 15 shows the results from an experiment to determine the percentage of migrating monocytes (CD14 primary cells) following treatment with 10 μg/mL of anti-MOSPD2 antibody (αMOSPD2), Natalizumab (anti-α4β1 antibody), Vedolizumab (anti-α4β7 antibody), or control antibody (IgG). These data demonstrate that treatment with anti-MOSPD2 antibody (αMOSPD2) resulted in decreased monocyte migration compared to Natalizumab and Vedolizumab. Statistical significance of ***p<0.001 by t-test.

### Example 13

### Generation of MOSPD2 and CD63 Double Expressing Cells for Immuno-Coprecipitation

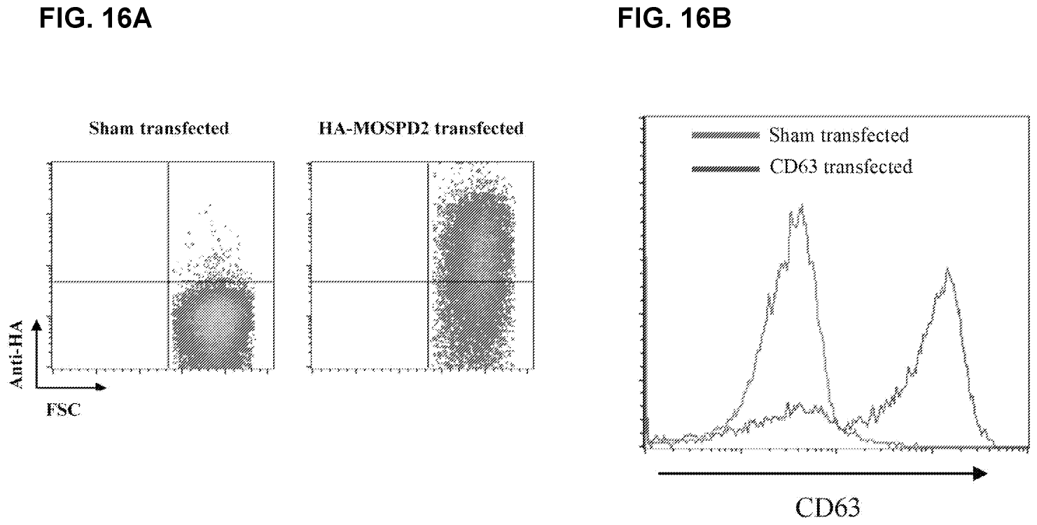

To validate yeast two-hybrid results, immuno-coprecipitation experiments were conducted. For that, HEK293 cells stably expressing HA-tagged human MOSPD2 were generated by transducing the cells with lentiviral particles expressing MOSPD2 in the presence of polybrene (8 μg/ml) and selecting transduced cells with medium containing puromycin (4 μg/ml). FIG. 16A shows positive staining for MOSPD2 in transduced cells expressing MOSPD2 compared with sham transduced cells. Then, the cells were transiently transfected for 48 hr with a plasmid expressing FLAG-tagged human CD63. FIG. 16B displays positive staining for CD63 in transfected compared with sham transfected cells.

### Example 14

### Generation of MOSPD2 and Integrin β2 Double Expressing Cells for Immuno-Coprecipitation

Integrins are heterodimer cell adhesion molecules that mediate cell-cell and cell-extracellular matrix (ECM) interactions. Studies have shown that integrins form complexes with CD63 that may influence cell migration (Barditchevski, 2001). Moreover, CD63 was found to associate with integrin β2 to induce activation of myeloid cells (Skubitz et al, 1996; 2000). Since MOSPD2 binds CD63, MOSPD2 may also bind integrin β2. To test that, HEK293 cells constitutively expressing HA-tagged MOSPD2 were transiently transfected with FLAG-tagged human integrin β2.

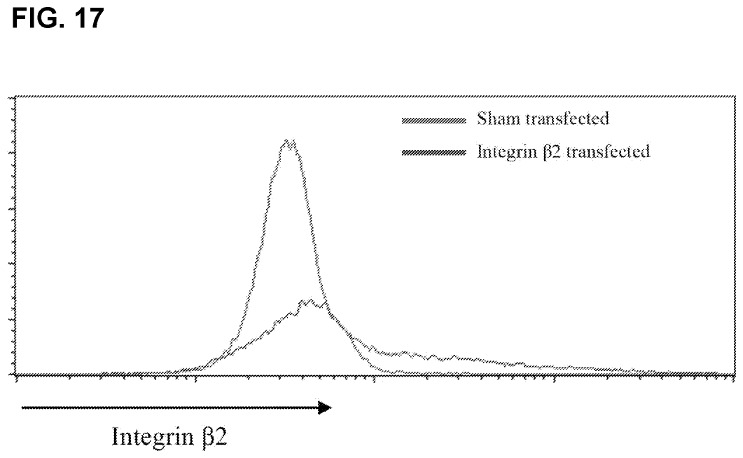

FIG. 17 shows flow cytometry analysis for expression of integrin β2 on stably expressing HA-tagged MOSPD2 expressing HEK293 cells that were sham transfected or transiently transfected with FLAG-tagged integrin β2 and stained with anti-integrin β2-PE. FIG. 17 displays positive staining for integrin β2 in transfected compared with sham transfected cells.

### Example 15

### Anti-MOSPD2 and Anti-CD63 Antibodies Promote Monocyte Binding to Endothelial Cell Adhesion Molecules

Migration of leukocytes from the circulation into inflamed tissues involves binding of integrins to adhesion molecules on endothelial cells and to the ECM. To learn how the interruption of MOSPD2 or CD63 will affect monocyte binding to the integrin 32 adhesion molecules ICAM-1 and VCAM-1, 48 well plates were washed with DPBS containing magnesium and calcium (wash buffer), coated with ICAM-1 or VCAM-1 (10 μg/ml) for 60 min at 37° C., washed and blocked with 0.5% BSA in DPBS for 60 min at 37° C. Wells were kept overnight with wash buffer in 4° C. CD14 monocytes pre-incubated with the indicated antibodies (10 μg/ml) for 45 min at 37° C. in starvation medium (RPMI 0.5% FCS) were seeded (10⁶/well) on the plates for 90 min. After 3 washes, 200 μl of staining solution was added for 10 min, washed 4 times with DDW and left to air dry. Next, 200 μl of extraction solution was added to each well for 10 min on an orbital shaker. 150 μl from each extracted sample was transferred to a 96-well microtiter plate and the plate was read at OD 560 nm in a plate reader.

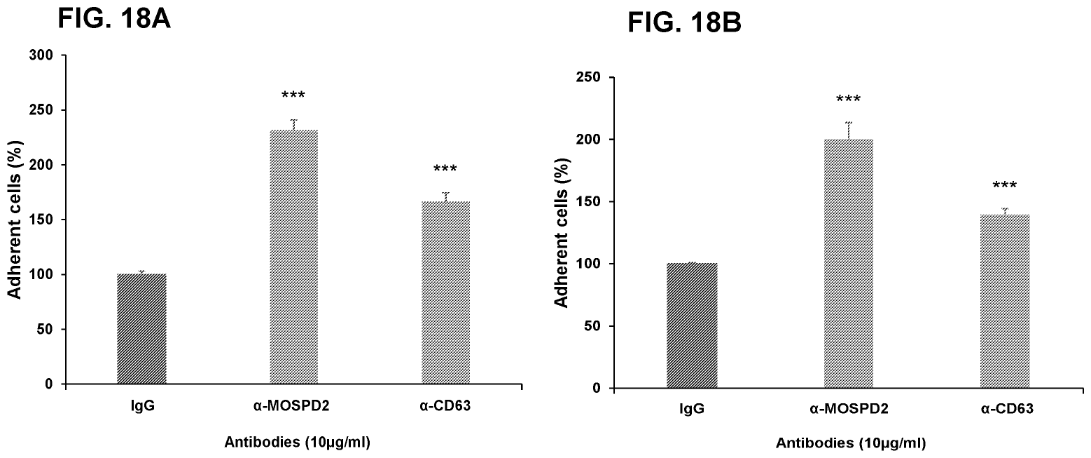

The results shown in FIG. 18A and FIG. 18B demonstrated that anti-MOSPD2 mAb or anti-CD63 mAb promote binding of monocytes to ICAM-1 and VCAM-1, adhesion molecules that are expressed on endothelial cells.

### Example 16

### Anti-MOSPD2 Antibody Induces Low to High Affinity Transition of LFA-1 on Monocytes

Leukocyte adhesion to endothelial cells and ECM is dependent on the transition of integrins from low to high affinity conformation. Since anti-MOSPD2 mAb induces signaling pathways associated with adhesion and migration, we assessed whether it can also instigate integrin change of conformation. The monoclonal antibody m24 recognizes LFA-1 (integrin αLβ2) only in its extended, high affinity conformation. Human primary monocytes or THP-1 monocytic line cells were starved for 4 or 24 hr respectively. Cells (0.5×10⁶/sample) were then re-suspend in 0.5 ml starvation medium and incubate for 30 min at 37° C. with IgG control or anti-MOSPD2 mAb (10 μg/ml) for 30 min in 37° C. in the presence of Alexa-Fluor 647 conjugated m24 (0.2 μg/sample). Cells were then washed and analyzed by flow cytometry.

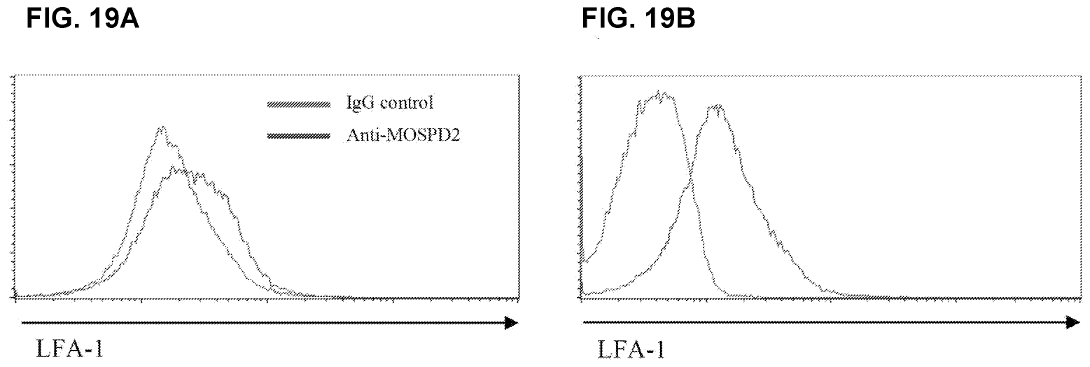

The results in FIG. 19A and FIG. 19B show that anti-MOSPD2 antibody induces the transition of LFA-1 from low to high affinity conformation.

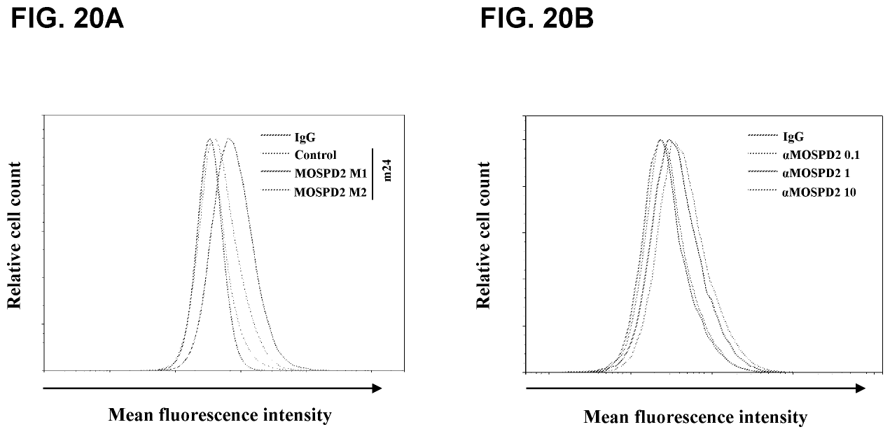

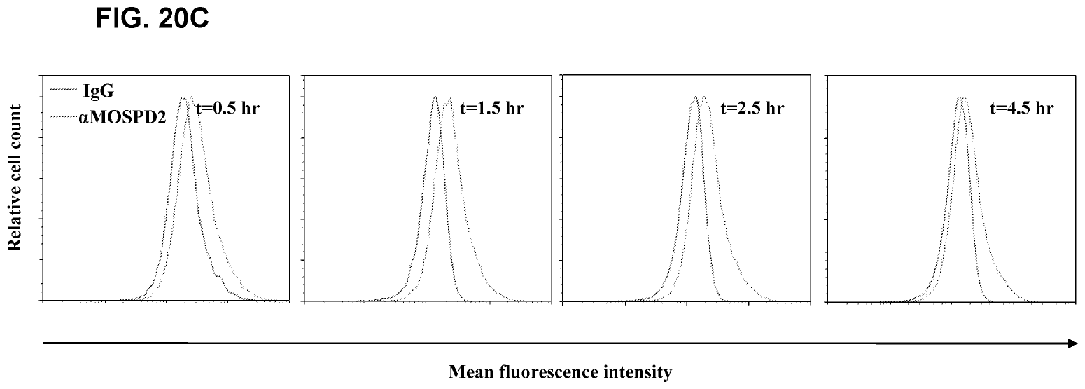

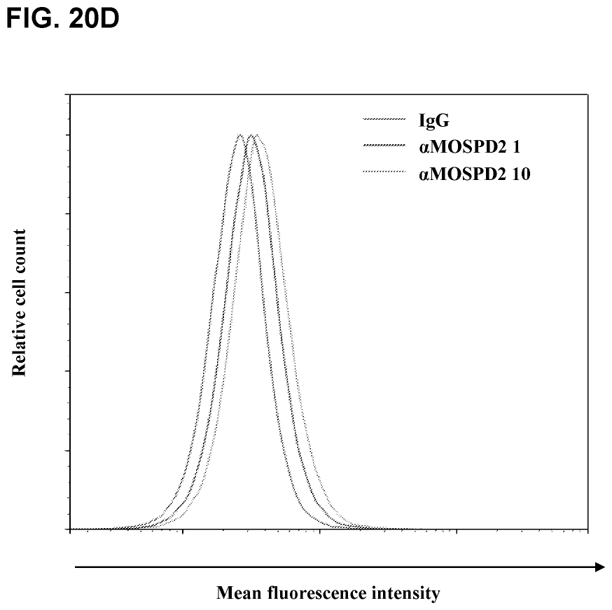

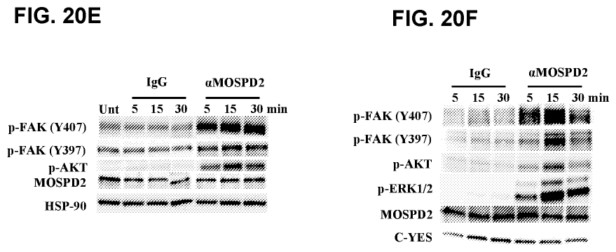

Additional data showing that MOSPD2 governs the conformation state of LFA-1 is provided in FIGS. 20A-20F.

FIG. 20A shows staining of CRISPR-Control or CRISPR-MOSPD2 M1 and M2 clones described in FIG. 3A with anti-LFA-1 clone m24, as detected by flow cytometry. CRISPR-Control cells were also stained with an isotype-control antibody (IgG).

FIG. 20B shows staining of THP-1 cells treated with isotype control antibody (IgG, 10 g/mL) or different doses of anti-MOSPD2 antibody (αMOSPD2, 0.1 g/mL, 1 g/mL, or 10 g/mL) with anti-LFA-1 clone m24, as detected by flow cytometry.

FIG. 20C shows flow cytometry results of THP-1 cells treated with isotype control antibody or anti-MOSPD2 antibody (αMOSPD2, 10 μg/ml) for the indicated times and stained with anti-LFA-1 clone m24.

FIG. 20D shows flow cytometry results of CD14+ monocytes treated with isotype control antibody (IgG, 10 g/mL) or different doses of anti-MOSPD2 antibody (αMOSPD2, 1 μg/mL or 10 μg/mL) and stained with anti-LFA-1 clone m24.

FIGS. 20E and 20F show representative western blot images from experiments to determine the presence of phosphorylated FAK (p-FAK at amino acid residues Y407 and Y397), phosphorylated AKT (p-AKT) and phosphorylated ERK1/2 (p-ERK1/2) of THP-1 (FIG. 20E) or CD14+ monocytes (FIG. 20F) treated with isotype control antibody (IgG) or anti-MOSPD2 antibody (αMOSPD2, 10 μg/ml) at the indicated time points. HSP-90 and C-YES were used as loading controls for FIGS. 20E and 20F, respectively.

### Example 17

### Anti-MOSPD2 Antibodies Prevent Tumor Metastasis

Previous data show that MOSPD2 is highly expressed in multiple solid tumors and cancer cell lines but has low/undetectable levels in healthy tissues. In vitro experiments have shown that silencing MOSPD2 in different breast cancer cell lines significantly inhibited cancer cell chemotaxis migration. Mechanistically, silencing MOSPD2 has been shown to profoundly abate phosphorylation events that are involved in breast tumor cell chemotaxis. In vivo, MOSPD2-silenced breast cancer cells exhibit marked impaired metastasis to the lungs. Taken together, these data suggest that targeting MOSPD2 using monoclonal antibodies may have a therapeutic effect on MOSPD2-expressing tumor cells for the treatment of cancer.

23 SCID-Beige female mice (7⁻⁹ weeks old) were intravenously (IV) injected with 1×10⁶ MDA-231 cells in order to induce lung metastasis. Mice were treated with Control Ab or anti-MOSPD2 mAb 25 mg/kg by intraperitoneal (IP) injection, on days (−4); (−2); 3, 10 and 17. 4 weeks from day 0 mice were sacrificed and lungs were fixed in formalin for further embedding in paraffin and H&E staining of 5 μM lung sections. The tumor area was calculated using a computer software (Imag Pro Plus) and the mean area (%) of 4⁻⁵ fields per mouse was calculated.

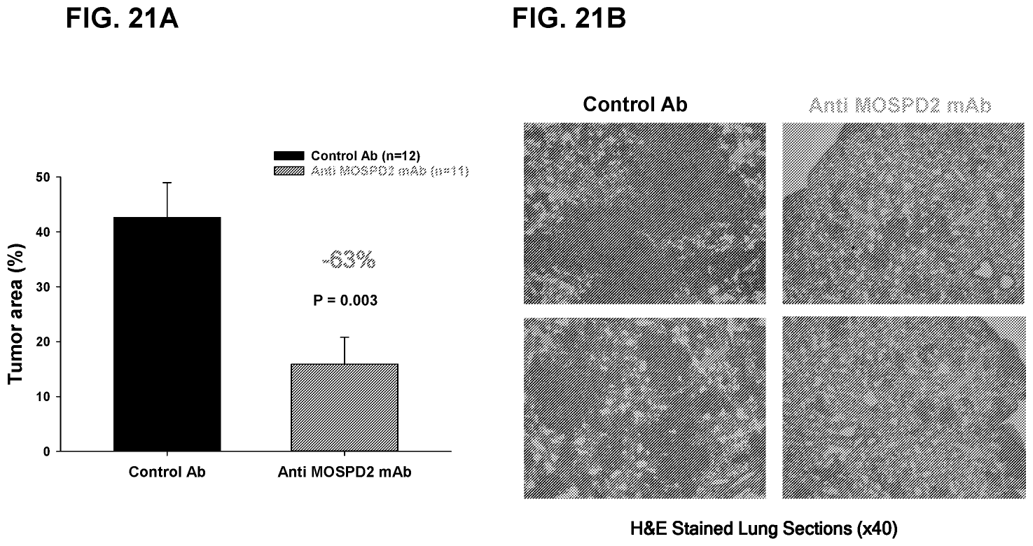

FIG. 21A shows tumor area (%) of anti-MOSPD2 antibody (Anti MOSPD2 mAb) treated mice (n=12) versus control antibody (Control Ab) treated mice (n=11). FIG. 21B shows representative pictures of lung H&E staining sections. Left side—control antibody treated. Right side—anti-MOSPD2 antibody treated. Moreover, anti-MOSPD2 antibody treatment significantly (p=0.003; t-test) decreased metastasis area by 63% in comparison to control antibody treated mice.

The following sequences in Table 1 are part of the present disclosure.

TABLE 1
Sequences

| SEQ ID NO: | Sequence |
|---|---|
| 1 | Homo sapiens motile sperm domain containing 2 |
| | (MOSPD2), protein variant 1 |
| | Met Ala Glu Asn His Ala Gln Asn Lys Ala Lys Leu Ile Ser |
| | Glu Thr Arg Arg Arg Phe Glu Ala Glu Tyr Val Thr Asp |
| | Lys Ser Asp Lys Tyr Asp Ala Arg Asp Val Glu Arg Leu |
| | Gln Gln Asp Asp Asn Trp Val Glu Ser Tyr Leu Ser Trp |
| | Arg His Asn Ile Val Asp Glu Thr Leu Lys Met Leu Asp |
| | Glu Ser Phe Gln Trp Arg Lys Glu Ile Ser Val Asn Asp Leu |
| | Asn Glu Ser Ser Ile Pro Arg Trp Leu Leu Glu Ile Gly Val |
| | Ile Tyr Leu His Gly Tyr Asp Lys Glu Gly Asn Lys Leu Phe |
| | Trp Ile Arg Val Lys Tyr His Val Lys Asp Gln Lys Thr Ile |
| | Leu Asp Lys Lys Lys Leu Ile Ala Phe Trp Leu Glu Arg Tyr |
| | Ala Lys Arg Glu Asn Gly Lys Pro Val Thr Val Met Phe |
| | Asp Leu Ser Glu Thr Gly Ile Asn Ser Ile Asp Met Asp Phe |
| | Val Arg Phe Ile Ile Asn Cys Phe Lys Val Tyr Tyr Pro Lys |
| | Tyr Leu Ser Lys Ile Val Ile Phe Asp Met Pro Trp Leu Met |
| | Asn Ala Ala Phe Lys Ile Val Lys Thr Trp Leu Gly Pro Glu |
| | Ala Val Ser Leu Leu Lys Phe Thr Ser Lys Asn Glu Val Gln |
| | Asp Tyr Val Ser Val Glu Tyr Leu Pro Pro His Met Gly Gly |
| | Thr Asp Pro Phe Lys Tyr Ser Tyr Pro Pro Leu Val Asp Asp |
| | Asp Phe Gln Thr Pro Leu Cys Glu Asn Gly Pro Ile Thr Ser |
| | Glu Asp Glu Thr Ser Ser Lys Glu Asp Ile Glu Ser Asp Gly |
| | Lys Glu Thr Leu Glu Thr Ile Ser Asn Glu Glu Gln Thr Pro |
| | Leu Leu Lys Lys Ile Asn Pro Thr Glu Ser Thr Ser Lys Ala |
| | Glu Glu Asn Glu Lys Val Asp Ser Lys Val Lys Ala Phe |
| | Lys Lys Pro Leu Ser Val Phe Lys Gly Pro Leu Leu His Ile |
| | Ser Pro Ala Glu Glu Leu Tyr Phe Gly Ser Thr Glu Ser Gly |
| | Glu Lys Lys Thr Leu Ile Val Leu Thr Asn Val Thr Lys Asn |
| | Ile Val Ala Phe Lys Val Arg Thr Thr Ala Pro Glu Lys Tyr |
| | Arg Val Lys Pro Ser Asn Ser Ser Cys Asp Pro Gly Ala Ser |
| | Val Asp Ile Val Val Ser Pro His Gly Gly Leu Thr Val Ser |
| | Ala Gln Asp Arg Phe Leu Ile Met Ala Ala Glu Met Glu |
| | Gln Ser Ser Gly Thr Gly Pro Ala Glu Leu Thr Gln Phe Trp |
| | Lys Glu Val Pro Arg Asn Lys Val Met Glu His Arg Leu |
| | Arg Cys His Thr Val Glu Ser Ser Lys Pro Asn Thr Leu Thr |
| | Leu Lys Asp Asn Ala Phe Asn Met Ser Asp Lys Thr Ser |
| | Glu Asp Ile Cys Leu Gln Leu Ser Arg Leu Leu Glu Ser Asn |
| | Arg Lys Leu Glu Asp Gln Val Gln Arg Cys Ile Trp Phe Gln |
| | Gln Leu Leu Leu Ser Leu Thr Met Leu Leu Leu Ala Phe |
| | Val Thr Ser Phe Phe Tyr Leu Leu Tyr Ser |
| 2 | Homo sapiens motile sperm domain containing 2 |
| | (MOSPD2), protein variant 2 |
| | MLDESFQWRKEISVNDLNESSIPRWLLEIGVIY |
| | LHGYDKEGNKLFWIRVKYHVKDQKTILDKKK |
| | LIAFWLERYAKRENGKPVTVMFDLSETGINSID |
| | MDFVRFIINCFKVYYPKYLSKIVIFDMPWLMNA |
| | AFKIVKTWLGPEAVSLLKFTSKNEVQDYVSVEY |
| | LPPHMGGTDPFKYSYPPLVDDDFQTPLCENGPIT |
| | SEDETSSKEDIESDGKETLETISNEEQTPLLKKINP |
| | TESTSKAEENEKVDSKVKAFKKPLSVFKGPLLHI |
| | SPAEELYFGSTESGEKKTLIVLTNVTKNIVAFKVR |
| | TTAPEKYRVKPSNSSCDPGASVDIVVSPHGGLT |
| | VSAQDRFLIMAAEMEQSSGTGPAELTQFWKEVP |
| | RNKVMEHRLRCHTVESSKPNTLTLKDNAFNMSD |
| | KTSEDICLQLSRLLESNRKLEDQVQRCIWFQQLLL |
| | SLTMLLLAFVTSFFYLLYS |
| 3 | Homo sapiens motile sperm domain containing 2 |
| | (MOSPD2), protein variant 3 |
| | MAENHAQNKAKLISETRRRFEAEYVTDKSDKY |
| | DARDVERLQQDDNWVESYLSWRHNIVDETLK |
| | MLDESFQWRKEISVNDLNESSIPRWLLEIGVIYL |
| | HGYDKEGNKLFWIRVKYHVKDQKTILDKKKLI |
| | AFWLERYAKRENGKPVTVMFDLSETGINSIDMD |
| | FVRFIINCFKVYYPKYLSKIVIFDMPWLMNAAFK |
| | IVKTWLGPEAVSLLKFTSKNEVQDYVSVEYLPP |
| | HMGGTDPFKYSYPPLVDDDFQTPLCENGPITSE |
| | DETSSKEDIESDGKETLETISNEEQTPLLKKINPT |
| | ESTSKAEENEKVDSKVKAFKKPLSVFKGPLLHIS |
| | PAEELYFGSTESGEKKTLIVLTNVTKNIVAFKVR |
| | TTAPEKYRVKPSNSSCDPGASVDIVVSPHGGLTV |
| | SAQDRFLIMAAEMEQSSGTGPAELTQFWKEVPR |
| | NKVMEHRLRCHTVESSKPNTLTLKDNAFNMSDK |
| | TSEDICLQFATSSCEMDCSPP |
| 4 | Homo sapiens motile sperm domain containing 2 |
| | (MOSPD2), protein variant X2 |
| | MAENHAQNKAKLISETRRRFEAEYVTDKSDKY |
| | DARDVERLQQDDNWVESYLSWRHNIVDETLK |
| | MLDESFQWRKEISVNDLNESSIPRWLLEIGVIYL |
| | HGYDKEGNKLFWIRVKYHVKDQKTILDKKKLI |
| | AFWLERYAKRENGKPVTVMFDLSETGINSIDMD |
| | FVRFIINCFKVYYPKYLSKIVIFDMPWLMNAAFK |
| | IVKTWLGPEAVSLLKFTSKNEVQDYVSVEYLPP |
| | HMGGTDPFKYSYPPLVDDDFQTPLCENGPITSED |
| | ETSSKEDIESDGKETLETISNEEQTPLLKKINPTES |
| | TSKAEENEKVDSKVKAFKKPLSVFKGPLLHISPA |
| | EELYFGSTESGEKKTLIVLTNVTKNIVAFKVRTT |
| | APEKYRVKPSNSSCDPGASVDIVVSPHGGLTVSA |
| | QDRFLIMAAEMEQSSGTGPAELTQFWKEVPRNK |
| | VMEHRLRCHTVESSKPNTLTLKDNAFNMSDKTS |
| | EDICLQYS |
| 5 | Homo sapiens motile sperm domain containing 2 |
| | (MOSPD2), transcript variant 1, mRNA |
| | Coding region 125-1678 |
| | 1 accgcctccc cctcccaccc ttctctgtct acctctgggc ggactgccg ggtgatgaga |
| | 61 tactcggtcg gcgacggtag aacgggcgac ggcgacaacc gcaatcacat ccacgacggt |
| | 121 gatcatggca gagaatcacg cccagaataa agccaagctc atctctgaga cccggaggag |
| | 181 gttcgaagct gagtatgtga cagataagtc agataaatat gatgcacgtg atgttgaaag |
| | 241 gctacaacaa gatgataact gggttgaaag ttacttatct tggagacata atattgtaga |
| | 301 tgaaacactg aagatgctcg atgagagttt tcagtggagg aaagaaattt ctgtcaatga |
| | 361 ccttaatgaa tcctccattc ccagatggtt attggaaatt ggtgttattt atctccatgg |
| | 421 ttatgacaaa gaaggtaaca aattgttctg gatcagggtg aagtatcatg taaaagacca |
| | 481 gaaaaccata ttggacaaaa agaagctcat agcattctgg ttggaacgtt atgctaagag |
| | 541 ggaaaatggg aaacctgtaa cagtgatgtt tgacctgtca gaaactggaa taaatagcat |
| | 601 tgacatggac tttgtacgct ttatcatcaa ctgctttaag gtttattacc ctaaatacct |
| | 661 ctcaaaaata gtgatctttg atatgccttg gttaatgaat gctgctttca aaattgtgaa |
| | 721 aacctggctt ggtccagaag cagtgagctt gttgaagttt acaagcaaaa atgaagtcca |
| | 781 ggactatgtc agtgtagaat acctgcctcc ccacatgggt ggaactgatc ctttcaagta |
| | 841 tagctatcca ccactagtag atgatgactt ccagacccca ctgtgtgaga atgggcctat |
| | 901 taccagtgag gatgaaactt caagtaaaga agacatagaa agtgatggca aagaaacatt |
| | 961 ggaaacaatt tctaatgaag aacaaacacc tcttcttaaa aagattaacc caaccgaatc |
| | 1021 tacttccaaa gcagaagaaa atgaaaaagt tgattcaaaa gtgaaagctt tcaagaaacc |
| | 1081 attgagtgta tttaaaggcc ccttactaca catcagccca gcagaagaac tgtactttgg |
| | 1141 aagtacagaa tccggagaga agaaaacctt aatagtgttg acaaatgtaa ctaaaaatat |
| | 1201 agtggcattt aaggtgagaa caacagctcc agaaaaatac agagtcaagc caagcaatag |
| | 1261 cagctgtgac ccgggtgcat cagtggatat agttgtgtct ccccatgggg gtttaacagt |
| | 1321 ctctgcccaa gaccgttttc tgataatggc tgcagaaatg gaacagtcat ctggcacagg |
| | 1381 cccagcagaa ttaactcagt tttggaaaga agttcccaga aacaaagtga tggaacatag |
| | 1441 gttaagatgc catactgttg aaagcagtaa accaaacact cttacgttaa aagacaatgc |
| | 1501 tttcaatatg tcagataaaa ccagtgaaga tatatgtcta caactcagtc gtttactaga |
| | 1561 aagcaatagg aagcttgaag accaagttca gcgttgtatc tggttccagc agctgctgct |
| | 1621 ttccttaaca atgctcttgc ttgcttttgt cacctctttc ttctatttat tgtacagtta |
| | 1681 aagaagtggt gccgggtagg aaccacggtt ccttcgtcca ttagttggaa aaagtaacag |
| | 1741 acctaaaact ctaccaagct actaaaaaca ttgcacatct gtgcttccta aaaggaaata |
| | 1801 tgcagcacgt ggaggggaac acatacatgt cttgaaaata aactgctaga ataaagaaat |
| | 1861 gctggagaaa ttgattataa gagactatag ctatttagta aagtaagtaa aggcatatcc |
| | 1921 attgtgtaaa ttaatagttt aaatataatt tattttttcc ttttgatctg aatactttta |
| | 1981 aagcttaagt tttatcgtgt aaatacatta gctaaactga aaagtataag taacatgctt |
| | 2041 tgttgcagcc aaaaaatgta atctgctttt ttatgacaga attattatag ctgagctgac |
| | 2101 ttactagctt ttctatacta tgtatataga agaacatgta tattgagaaa gaaaacatac |
| | 2161 ttatatagag gaatttatgt aaccatgact ttgtaatttt gagaattcct cccagtgatg |
| | 2221 gtcagtattc ttttggaatg taaaccgatt taatgccaaa ccaccttaac ctttgtttct |
| | 2281 cagtgttcct taacagcctg ccttttatta atctcaggct tttttatgaa cactctcatt |
| | 2341 tcagtagaat ttggaaaact aagcgtggtt ggaatttctt tgaattctgt tagtaatgcc |
| | 2401 caaaagaaaa gtctcaagca gtccccctat ccagtcattt ttatggagtt tcatgttgtc |
| | 2461 cactatagct ggacactgaa ccttttgcct aatttattat aaaggcctga ccctctattg |
| | 2521 tcccatcttc acccccattc cagagcagag gagtctctgt ggaccatgaa ttgcactgtc |
| | 2581 tccctcctca tttctaaatg aaaggtatta gatataaatt tttttgaaag gttagttgtt |
| | 2641 tgagatgcta agcaggataa taaatttaga ttttaaaatg ttccctgtaa aagtcagccc |
| | 2701 atgacaagga aatttacaaa atactagagt atctagaagg gtgaaaacaa aaaaaaataa |
| | 2761 aaagaaacac agacgcccag gtgtcagctc tccgtttaaa gaatgaaaaa tgtaactcat |
| | 2821 gatgatctgt gaaaccttca aactaggacc aattgactta cttgatattc tgcctttgat |
| | 2881 atggtagtac ccacccggta ttcctaaaat cctaaaaaga tacaccttgc agtagcagag |
| | 2941 gcaatgacat gagtttgttt tctcattaat atgaccagtt tgggtctatg ttggttcaca |
| | 3001 tgtacatcta ctttatatga aagaaaaaac agttgtctgc ctgtaaaatg ttgagtttcg |
| | 3061 attgagccat gtttggagat tttattacta ttctgaaggg tagtgttgtt ggttttcatc |
| | 3121 ttcaagaagt tgattccaaa actgagttat gaagaatgat ataacagttc cttcaaaatt |
| | 3181 ggcctaggaa ataaaacctt aaaaggacaa aaaaaaaaa |
| 6 | Homo sapiens motile sperm domain containing 2 |
| | (MOSPD2), transcript variant 2, mRNA |
| | Coding region 278-1642 |
| | 1 agtcacaata ataggtactt aaaaacatgt catatgatag gaaactagaa taacacacct |
| | 61 ctaaataatc catggattac caacttctca gcaatggtgt aaattcttct gctaaataac |
| | 121 ccgtgggtta aagaggacat cacattggaa attccagagt acttaaaata agtcagataa |
| | 181 atatgatgca cgtgatgttg aaaggctaca acaagatgat aactgggttg aaagttactt |
| | 241 atcttggaga cataatattg tagatgaaac actgaagatg ctcgatgaga gttttcagtg |
| | 301 gaggaaagaa atttctgtca atgaccttaa tgaatcctcc attcccagat ggttattgga |
| | 361 aattggtgtt atttatctcc atggttatga caaagaaggt aacaaattgt tctggatcag |
| | 421 ggtgaagtat catgtaaaag accagaaaac catattggac aaaaagaagc tcatagcatt |
| | 481 ctggttggaa cgttatgcta agagggaaaa tgggaaacct gtaacagtga tgtttgacct |
| | 541 gtcagaaact ggaataaata gcattgacat ggactttgta cgctttatca tcaactgctt |
| | 601 taaggtttat taccctaaat acctctcaaa aatagtgatc tttgatatgc cttggttaat |
| | 661 gaatgctgct ttcaaaattg tgaaaacctg gcttggtcca gaagcagtga gcttgttgaa |
| | 721 gtttacaagc aaaaatgaag tccaggacta tgtcagtgta gaatacctgc ctccccacat |
| | 781 gggtggaact gatcctttca agtatagcta tccaccacta gtagatgatg acttccagac |
| | 841 cccactgtgt gagaatgggc ctattaccag tgaggatgaa acttcaagta aagaagacat |
| | 901 agaaagtgat ggcaaagaaa cattggaaac aatttctaat gaagaacaaa cacctcttct |
| | 961 taaaaagatt aacccaaccg aatctacttc caaagcagaa gaaaatgaaa aagttgattc |
| | 1021 aaaagtgaaa gctttcaaga aaccattgag tgtatttaaa ggccccttac tacacatcag |
| | 1081 cccagcagaa gaactgtact ttggaagtac agaatccgga gagaagaaaa ccttaatagt |
| | 1141 gttgacaaat gtaactaaaa atatagtggc atttaaggtg agaacaacag ctccagaaaa |
| | 1201 atacagagtc aagccaagca atagcagctg tgacccgggt gcatcagtgg atatagttgt |
| | 1261 gtctccccat gggggtttaa cagtctctgc ccaagaccgt tttctgataa tggctgcaga |
| | 1321 aatggaacag tcatctggca caggcccagc agaattaact cagttttgga aagaagttcc |
| | 1381 cagaaacaaa gtgatggaac ataggttaag atgccatact gttgaaagca gtaaaccaaa |
| | 1441 cactcttacg ttaaaagaca atgctttcaa tatgtcagat aaaaccagtg aagatatatg |
| | 1501 tctacaactc agtcgtttac tagaaagcaa taggaagctt gaagaccaag ttcagcgttg |
| | 1561 tatctggttc cagcagctgc tgctttcctt aacaatgctc ttgcttgctt ttgtcacctc |
| | 1621 tttcttctat ttattgtaca gttaaagaag tggtgccggg taggaaccac ggttccttcg |
| | 1681 tccattagtt ggaaaaagta acagacctaa aactctacca agctactaaa aacattgcac |
| | 1741 atctgtgctt cctaaaagga aatatgcagc acgtggaggg gaacacatac atgtcttgaa |
| | 1801 aataaactgc tagaataaag aaatgctgga gaaattgatt ataagagact atagctattt |
| | 1861 agtaaagtaa gtaaaggcat atccattgtg taaattaata gtttaaatat aatttatttt |
| | 1921 ttccttttga tctgaatact tttaaagctt aagttttatc gtgtaaatac attagctaaa |
| | 1981 ctgaaaagta taagtaacat gctttgttgc agccaaaaaa tgtaatctgc ttttttatga |
| | 2041 cagaattatt atagctgagc tgacttacta gcttttctat actatgtata tagaagaaca |
| | 2101 tgtatattga gaaagaaaac atacttatat agaggaattt atgtaaccat gactttgtaa |
| | 2161 ttttgagaat tcctcccagt gatggtcagt attcttttgg aatgtaaacc gatttaatgc |
| | 2221 caaaccacct taacctttgt ttctcagtgt tccttaacag cctgcctttt attaatctca |
| | 2281 ggctttttta tgaacactct catttcagta gaatttggaa aactaagcgt ggttggaatt |
| | 2341 tctttgaatt ctgttagtaa tgcccaaaag aaaagtctca agcagtcccc ctatccagtc |
| | 2401 atttttatgg agtttcatgt tgtccactat agctggacac tgaacctttt gcctaattta |
| | 2461 ttataaaggc ctgaccctct attgtcccat cttcaccccc attccagagc agaggagtct |
| | 2521 ctgtggacca tgaattgcac tgtctccctc ctcatttcta aatgaaaggt attagatata |
| | 2581 aatttttttg aaaggttagt tgtttgagat gctaagcagg ataataaatt tagattttaa |
| | 2641 aatgttccct gtaaaagtca gcccatgaca aggaaattta caaaatacta gagtatctag |
| | 2701 aagggtgaaa acaaaaaaaa ataaaaagaa acacagacgc ccaggtgtca gctctccgtt |
| | 2761 taaagaatga aaaatgtaac tcatgatgat ctgtgaaacc ttcaaactag gaccaattga |
| | 2821 cttacttgat attctgcctt tgatatggta gtacccaccc ggtattccta aaatcctaaa |
| | 2881 aagatacacc ttgcagtagc agaggcaatg acatgagttt gttttctcat taatatgacc |
| | 2941 agtttgggtc tatgttggtt cacatgtaca tctactttat atgaaagaaa aaacagttgt |
| | 3001 ctgcctgtaa aatgttgagt ttcgattgag ccatgtttgg agattttatt actattctga |
| | 3061 agggtagtgt tgttggtttt catcttcaag aagttgattc caaaactgag ttatgaagaa |
| | 3121 tgatataaca gttccttcaa aattggccta ggaaataaaa ccttaaaagg acaaaaaaaa |
| | 3181 aaa |
| 7 | Homo sapiens motile sperm domain containing 2 |
| | (MOSPD2), transcript variant 3, mRNA |
| | Coding region 125-1582 |
| | 1 accgcctccc cctcccaccc ttctctgtct acctctgggc gggactgccg ggtgatgaga |
| | 61 tactcggtcg gcgacggtag aacgggcgac ggcgacaacc gcaatcacat ccacgacggt |
| | 121 gatcatggca gagaatcacg cccagaataa agccaagctc atctctgaga cccggaggag |
| | 181 gttcgaagct gagtatgtga cagataagtc agataaatat gatgcacgtg atgttgaaag |
| | 241 gctacaacaa gatgataact gggttgaaag ttacttatct tggagacata atattgtaga |
| | 301 tgaaacactg aagatgctcg atgagagttt tcagtggagg aaagaaattt ctgtcaatga |
| | 361 ccttaatgaa tcctccattc ccagatggtt attggaaatt ggtgttattt atctccatgg |
| | 421 ttatgacaaa gaaggtaaca aattgttctg gatcagggtg aagtatcatg taaaagacca |
| | 481 gaaaaccata ttggacaaaa agaagctcat agcattctgg ttggaacgtt atgctaagag |
| | 541 ggaaaatggg aaacctgtaa cagtgatgtt tgacctgtca gaaactggaa taaatagcat |
| | 601 tgacatggac tttgtacgct ttatcatcaa ctgctttaag gtttattacc ctaaatacct |
| | 661 ctcaaaaata gtgatctttg atatgccttg gttaatgaat gctgctttca aaattgtgaa |
| | 721 aacctggctt ggtccagaag cagtgagctt gttgaagttt acaagcaaaa atgaagtcca |
| | 781 ggactatgtc agtgtagaat acctgcctcc ccacatgggt ggaactgatc ctttcaagta |
| | 841 tagctatcca ccactagtag atgatgactt ccagacccca ctgtgtgaga atgggcctat |
| | 901 taccagtgag gatgaaactt caagtaaaga agacatagaa agtgatggca aagaaacatt |
| | 961 ggaaacaatt tctaatgaag aacaaacacc tcttcttaaa aagattaacc caaccgaatc |
| | 1021 tacttccaaa gcagaagaaa atgaaaaagt tgattcaaaa gtgaaagctt tcaagaaacc |
| | 1081 attgagtgta tttaaaggcc ccttactaca catcagccca gcagaagaac tgtactttgg |
| | 1141 aagtacagaa tccggagaga agaaaacctt aatagtgttg acaaatgtaa ctaaaaatat |
| | 1201 agtggcattt aaggtgagaa caacagctcc agaaaaatac agagtcaagc caagcaatag |
| | 1261 cagctgtgac ccgggtgcat cagtggatat agttgtgtct ccccatgggg gtttaacagt |
| | 1321 ctctgcccaa gaccgttttc tgataatggc tgcagaaatg gaacagtcat ctggcacagg |
| | 1381 cccagcagaa ttaactcagt tttggaaaga agttcccaga aacaaagtga tggaacatag |
| | 1441 gttaagatgc catactgttg aaagcagtaa accaaacact cttacgttaa aagacaatgc |
| | 1501 tttcaatatg tcagataaaa ccagtgaaga tatatgtcta caatttgcca cctccagctg |
| | 1561 tgaaatggac tgcagtccac cctaagtact gtgcacagta tctccctgtg tgtgtgcaca |
| | 1621 gtggcttccc cttacatggt agatttttgg ccttaatata atctaatccc aaagtagttg |
| | 1681 tgtatgtttt ctgttccttg gcaaataaat gaagaaataa ttagccaaga ttgaaaatgt |
| | 1741 attgtcctaa cggtgtccct ttaatgtttc atatgaaaaa ttatgttgac ccactaaaat |
| | 1801 atccttgctc aatgtctggt cagttgaatt taataacata tcttgttaat gtttgtgtgt |
| | 1861 ctattaaatg tgactaagca ggattactga aaattcacta taaaatcaaa ggcatctaaa |
| | 1921 cgtttgtact tgtcttgatt aatcatatat ttacacttga tttttttctg tcttcatttg |
| | 1981 tttttattta atcataattg catgattttt ttggtactct aatcagtaat tttattttta |
| | 2041 atcatgtcat tacctattca tgaccaaatt accaaggaac caacatttag atttagatat |
| | 2101 ttgttttcac ttaggaatgg aaattaatag attttccatg aaagcattag tgaaatatca |
| | 2161 ttaccttgat ctgcaagtag cctaaaaatg cgattgctgg taaacctggc ctcaaatttc |
| | 2221 atactaccat aactgttttt atatattgcc actaattttg actggattta atagcacttt |
| | 2281 attgtacaac tacaaaaaaa aatatattcc tagaattgtt gccagtgtaa |
| 8 | Homo sapiens motile sperm domain containing 2 |
| | (MOSPD2), transcript variant X2, mRNA |
| | Coding region 125-1549 |
| | 1 accgcctccc cctcccaccc ttctctgtct acctctgggc gggactgccg ggtgatgaga |
| | 61 tactcggtcg gcgacggtag aacgggcgac ggcgacaacc gcaatcacat ccacgacggt |
| | 121 gatcatggca gagaatcacg cccagaataa agccaagctc atctctgaga cccggaggag |
| | 181 gttcgaagct gagtatgtga cagataagtc agataaatat gatgcacgtg atgttgaaag |
| | 241 gctacaacaa gatgataact gggttgaaag ttacttatct tggagacata atattgtaga |
| | 301 tgaaacactg aagatgctcg atgagagttt tcagtggagg aaagaaattt ctgtcaatga |
| | 361 ccttaatgaa tcctccattc ccagatggtt attggaaatt ggtgttattt atctccatgg |
| | 421 ttatgacaaa gaaggtaaca aattgttctg gatcagggtg aagtatcatg taaaagacca |
| | 481 gaaaaccata ttggacaaaa agaagctcat agcattctgg ttggaacgtt atgctaagag |
| | 541 ggaaaatggg aaacctgtaa cagtgatgtt tgacctgtca gaaactggaa taaatagcat |
| | 601 tgacatggac tttgtacgct ttatcatcaa ctgctttaag gtttattacc ctaaatacct |
| | 661 ctcaaaaata gtgatctttg atatgccttg gttaatgaat gctgctttca aaattgtgaa |
| | 721 aacctggctt ggtccagaag cagtgagctt gttgaagttt acaagcaaaa atgaagtcca |
| | 781 ggactatgtc agtgtagaat acctgcctcc ccacatgggt ggaactgatc ctttcaagta |
| | 841 tagctatcca ccactagtag atgatgactt ccagacccca ctgtgtgaga atgggcctat |
| | 901 taccagtgag gatgaaactt caagtaaaga agacatagaa agtgatggca aagaaacatt |
| | 961 ggaaacaatt tctaatgaag aacaaacacc tcttcttaaa aagattaacc caaccgaatc |
| | 1021 tacttccaaa gcagaagaaa atgaaaaagt tgattcaaaa gtgaaagctt tcaagaaacc |
| | 1081 attgagtgta tttaaaggcc ccttactaca catcagccca gcagaagaac tgtactttgg |
| | 1141 aagtacagaa tccggagaga agaaaacctt aatagtgttg acaaatgtaa ctaaaaatat |
| | 1201 agtggcattt aaggtgagaa caacagctcc agaaaaatac agagtcaagc caagcaatag |
| | 1261 cagctgtgac ccgggtgcat cagtggatat agttgtgtct ccccatgggg gtttaacagt |
| | 1321 ctctgcccaa gaccgttttc tgataatggc tgcagaaatg gaacagtcat ctggcacagg |
| | 1381 cccagcagaa ttaactcagt tttggaaaga agttcccaga aacaaagtga tggaacatag |
| | 1441 gttaagatgc catactgttg aaagcagtaa accaaacact cttacgttaa aagacaatgc |
| | 1501 tttcaatatg tcagataaaa ccagtgaaga tatatgtcta caatacagtt aaagaagtgg |
| | 1561 tgccgggtag gaaccacggt tccttcgtcc attagttgga aaaagtaaca gacctaaaac |
| | 1621 tctaccaagc tactaaaaac attgcacatc tgtgcttcct aaaaggaaat atgcagcacg |
| | 1681 tggaggggaa cacatacatg tcttgaaaat aaactgctag aataaagaaa tgctggagaa |
| | 1741 attgattata agagactata gctatttagt aaagtaagta aaggcatatc cattgtgtaa |
| | 1801 attaatagtt taaatataat ttattttttc cttttgatct gaatactttt aaagcttaag |
| | 1861 ttttatcgtg taaatacatt agctaaactg aaaagtataa gtaacatgct ttgttgcagc |
| | 1921 caaaaaatgt aatctgcttt tttatgacag aattattata gctgagctga cttactagct |
| | 1981 tttctatact atgtatatag aagaacatgt atattgagaa agaaaacata cttatataga |
| | 2041 ggaatttatg taaccatgac tttgtaattt tgagaattcc tcccagtgat ggtcagtatt |
| | 2101 cttttggaat gtaaaccgat ttaatgccaa accaccttaa cctttgtttc tcagtgttcc |
| | 2161 ttaacagcct gccttttatt aatctcaggc ttttttatga acactctcat ttcagtagaa |
| | 2221 tttggaaaac taagcgtggt tggaatttct ttgaattctg ttagtaatgc ccaaaagaaa |
| | 2281 agtctcaagc agtcccccta tccagtcatt tttatggagt ttcatgttgt ccactatagc |
| | 2341 tggacactga accttttgcc taatttatta taaaggcctg accctctatt gtcccatctt |
| | 2401 cacccccatt ccagagcaga ggagtctctg tggaccatga attgcactgt ctccctcctc |
| | 2461 atttctaaat gaaaggtatt agatataaat ttttttgaaa ggttagttgt ttgagatgct |
| | 2521 aagcaggata ataaatttag attttaaaat gttccctgta aaagtcagcc catgacaagg |
| | 2581 aaatttacaa aatactagag tatctagaag ggtgaaaaca aaaaaaaata aaaagaaaca |
| | 2641 cagacgccca ggtgtcagct ctccgtttaa agaatgaaaa atgtaactca tgatgatctg |
| | 2701 tgaaaccttc aaactaggac caattgactt acttgatatt ctgcctttga tatggtagta |
| | 2761 cccacccggt attcctaaaa tcctaaaaag atacaccttg cagtagcaga ggcaatgaca |
| | 2821 tgagtttgtt ttctcattaa tatgaccagt ttgggtctat gttggttcac atgtacatct |
| | 2881 actttatatg aaagaaaaaa cagttgtctg cctgtaaaat gttgagtttc gattgagcca |
| | 2941 tgtttggaga ttttattact attctgaagg gtagtgttgt tggttttcat cttcaagaag |
| | 3001 ttgattccaa aactgagtta tgaagaatga tataacagtt ccttcaaaat tggcctagga |
| | 3061 aataaaacct taaaaggaca ctggtgtgct actttgtctt aatttgggct tttctgtttc |
| | 3121 agtttgccac ctccagctgt gaaatggact gcagtccacc ctaagtactg tgcacagtat |
| | 3181 ctccctgtgt gtgtgcacag tggcttcccc ttacatggta gatttttggc cttaatataa |
| | 3241 tctaatccca aagtagttgt gtatgttttc tgttccttgg caaataaatg aagaaataat |
| | 3301 tagccaagat tgaaaatgta ttgtcctaac ggtgtccctt taatgtttca tatgaaaaat |
| | 3361 tatgttgacc cactaaaata tccttgctca atgtctggtc agttgaattt aataacatat |
| | 3421 cttgttaatg tttgtgtgtc tattaaatgt gactaagcag gattactgaa aattcactat |
| | 3481 aaaatcaaag gcatctaaac gtttgtactt gtcttgatta atcatatatt tacacttgat |
| | 3541 ttttttctgt cttcatttgt ttttatttaa tcataattgc atgatttttt tggtactcta |
| | 3601 atcagtaatt ttatttttaa tcatgtcatt acctattcat gaccaaatta ccaaggaacc |
| | 3661 aacatttaga tttagatatt tgttttcact taggaatgga aattaataga ttttccatga |
| | 3721 aagcattagt gaaatatcat taccttgatc tgcaagtagc ctaaaaatgc gattgctggt |
| | 3781 aaacctggcc tcaaatttca tactaccata actgttttta tatattgcca ctaattttga |
| | 3841 ctggatttaa tagcacttta ttgtacaact acaaaaaaaa atatattcct agaattgttg |
| | 3901 ccagtgtaa |
| 9 | Integrin beta-2 isoform 1 precursor [Homo sapiens] |
| | 1 mlglrpplla lvgllslgcv lsqectkfkv sscreciesg pgctwcqkln ftgpgdpdsi |
| | 61 rcdtrpqllm rgcaaddimd ptslaetqed hnggqkqlsp qkvtlylrpg qaaafnvtfr |
| | 121 rakgypidly ylmdlsysml ddlrnvkklg gdllralnei tesgrigfgs fvdktvlpfv |
| | 181 nthpdklrnp cpnkekecqp pfafrhvlkl tnnsnqfqte vgkqlisgnl dapeggldam |
| | 241 mqvaacpeei gwrnvtrllv fatddgfhfa gdgklgailt pndgrchled nlykrsnefd |
| | 301 ypsvgqlahk laenniqpif avtsrmvkty eklteiipks avgelsedss nvvhliknay |
| | 361 nklssrvfld hnalpdtlkv tydsfcsngv thrnqprgdc dgvqinvpit fqvkvtatec |
| | 421 iqeqsfvira lgftdivtvq vlpqcecrer dqsrdrslch gkgflecgic rcdtgyigkn |
| | 481 cecqtqgrss qelegscrkd nnsiicsglg devcgqclch tsdvpgkliy gqycecdtin |
| | 541 ceryngqveg gpgrglcfcg kcrchpgfeg sacqcertte gclnprrvec sgrgrcrenv |
| | 601 cechsgyqlp lcqecpgcps pcgkyiscae clkfekgpfg kncsaacpgl qlsnnpvkgr |
| | 661 tckerdsegc wvaytleqqd gmdryliyvd esrecvagpn iaaivggtva givligilll |
| | 721 viwkalihls dlreyrrfek eklksqwnnd nplfksattt vmnpkfaes |
| 10 | Integrin beta-2 isoform 2 [Homo sapiens] |
| | 1 mrgcaaddim dptslaetqe dhnggqkqls pqkvtlylrp gqaaafnvtf rrakgypidl |
| | 61 yylmdlsysm lddlrnvkkl ggdllralne itesgrigfg sfvdktvlpf vnthpdklrn |
| | 121 pcpnkekecq ppfafrhvlk ltnnsnqfqt evgkqlisgn ldapegglda mmqvaacpee |
| | 181 igwrnvtrll vfatddgfhf agdgklgail tpndgrchle dnlykrsnef dypsvgqlah |
| | 241 klaenniqpi favtsrmvkt yeklteiipk savgelseds snvvhlikna ynklssrvfl |
| | 301 dhnalpdtlk vtydsfcsng vthrnqprgd cdgvqinvpi tfqvkvtate ciqeqsfvir |
| | 361 algftdivtv qvlpqcecrc rdqsrdrslc hgkgflecgi crcdtgyigk ncecqtqgrs |
| | 421 sqelegscrk dnnsiicsgl gdevegqclc htsdvpgkli ygqycecdti nceryngqvc |
| | 481 ggpgrglcfc gkcrchpgfe gsacqcertt egclnprrve csgrgrcren vcechsgyql |
| | 541 plcqecpgcp spcgkyisca eclkfekgpf gkncsaacpg lqlsnnpvkg rtckerdseg |
| | 601 cwvaytleqq dgmdryliyv desrecvagp niaaivggtv agivligill lviwkalihl |
| | 661 sdlreyrrfe keklksqwnn dnplfksatt tvmnpkfaes |
| 11 | Integrin beta-2 isoform X1 [Homo sapiens] |
| | 1 mrgcaaddim dptslaetqe dhnggqkqls pqkvtlylrp gqaaafnvtf rrakgypidl |
| | 61 yylmdlsysm lddlrnvkkl ggdllralne itesgrigfg sfvdktvlpf vnthpdklrn |
| | 121 pcpnkekecq ppfafrhvlk ltnnsnqfqt evgkqlisgn ldapegglda mmqvaacpee |
| | 181 igwrnvtrll vfatddgfhf agdgklgail tpndgrchle dnlykrsnef dypsvgqlah |
| | 241 klaenniqpi favtsrmvkt yeklteiipk savgelseds snvvhlikna ynklssrvfl |
| | 301 dhnalpdtlk vtydsfcsng vthrnqprgd cdgvqinvpi tfqvkvtate ciqeqsfvir |
| | 361 algftdivtv qvlpqcecrc rdqsrdrslc hgkgflecgi crcdtgyigk ncecqtqgrs |
| | 421 sqelegscrk dnnsiicsgl gdcvcgqclc htsdvpgkli ygqycecdti nceryngqvc |
| | 481 ggpgrglcfc gkcrchpgfe gsacqcertt egclnprrve csgrgrcren vcechsgyql |
| | 541 plcqecpgcp spcgkyisca eclkfekgpf gkncsaacpg lqlsnnpvkg rtckerdseg |
| | 601 cwvaytleqq dgmdryliyv desrecvagp niaaivggtv agivligill lviwkalihl |
| | 661 sdlreyrrfe keklksqwnn dnplfksatt tvmnpkfaes |
| 12 | CD63, partial, homo sapiens |
| | 1 maveggmkcv kfllyvllla fcacavglia vgvgaqlvls qtiiqgatpg sllpvviiav |
| | 61 gvflflvafv gccgackeny clmitfaifl slimlvevaa aiagyvfrdk vmsefnnnfr |
| | 121 qqmenypknn htasildrmq adfkccgaan ytdwekipsm sknrvpdscc idvtvgcgin |
| | 181 fnekaihkeg cvekiggwlr knvlvvaaaa lgiafvevlg ivfacclvks irsgyevm |

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-121. (canceled)

122. A method of inhibiting migration of an inflammatory cell, comprising contacting the inflammatory cell with (i) an agent capable of modulating the interaction of motile sperm domain containing protein 2 (MOSPD2) and CD63, or (ii) an agent capable of modulating the interaction of MOSPD2 and integrin β2.

123. The method of claim 122, wherein the agent capable of modulating the interaction of MOSPD2 and CD63 is an anti-MOSPD2 antibody, anti-CD63 antibody, or antigen binding fragment thereof.

124. The method of claim 122, wherein the agent capable of modulating the interaction of MOSPD2 and integrin β2 is an anti-MOSPD2 antibody, anti-integrin β2 antibody, or antigen binding fragment thereof.

125. The method of claim 122, wherein the inflammatory cell is a myeloid cell.

126. The method of claim 125, wherein the myeloid cell is a monocyte.

127. A method of treating an inflammatory disease or disorder, comprising administering (i) an agent capable of modulating the interaction of MOSPD2 and CD63, or (ii) an agent capable of modulating the interaction of MOSPD2 and integrin β2, to a subject in need thereof.

128. The method of claim 127, wherein the agent capable of modulating the interaction of MOSPD2 and CD63 is an anti-MOSPD2 antibody, anti-CD63 antibody, or antigen binding fragment thereof.

129. The method of claim 127, wherein the agent capable of modulating the interaction of MOSPD2 and integrin β2 is an anti-MOSPD2 antibody, anti-integrin β2 antibody, or antigen binding fragment thereof.

130. A method of treating or preventing cancer metastasis, comprising administering (i) an agent capable of modulating the interaction of MOSPD2 and CD63, or (ii) an agent capable of modulating the interaction of MOSPD2 and integrin β2, to a subject in need thereof.

131. The method of claim 130, wherein the agent capable of modulating the interaction of MOSPD2 and CD63 is an anti-MOSPD2 antibody, anti-CD63 antibody, or antigen binding fragment thereof.

132. The method of claim 130, wherein the agent capable of modulating the interaction of MOSPD2 and integrin β2 is an anti-MOSPD2 antibody, anti-integrin β2 antibody, or antigen binding fragment thereof.

133. A method of treating an inflammatory disease or disorder, comprising administering (i) an inhibitor of MOSPD2, and (ii) an inhibitor of CD63 or an inhibitor of integrin β2, to a subject in need thereof.

134. The method of claim 133, wherein the inhibitor of MOSPD2 is an anti-MOSPD2 antibody or antigen binding fragment thereof.

135. The method of claim 133, wherein the inhibitor of CD63 is an anti-CD63 antibody or antigen binding fragment thereof.

136. The method of claim 133, wherein the inhibitor of integrin β2 is an anti-integrin β2 antibody or antigen binding fragment thereof.

137. The method of claim 133, comprising administering (i) an inhibitor of MOSPD2, (ii) an inhibitor of CD63, and (iii) an inhibitor of integrin β2, to a subject in need thereof.

138. A method of treating or preventing cancer metastasis, comprising administering (i) an inhibitor of MOSPD2, and (ii) an inhibitor of CD63 or an inhibitor of integrin β2, to a subject in need thereof.

139. The method of claim 138, wherein the inhibitor of MOSPD2 is an anti-MOSPD2 antibody or antigen binding fragment thereof.

140. The method of claim 138, wherein the inhibitor of CD63 is an anti-CD63 antibody or antigen binding fragment thereof.

141. The method of claim 138, wherein the inhibitor of integrin β2 is an anti-integrin β2 antibody or antigen binding fragment thereof.

142. The method of claim 138, comprising administering (i) an inhibitor of MOSPD2, (ii) an inhibitor of CD63, and (iii) an inhibitor of integrin β2, to a subject in need thereof.
